(19) United States
(12) Patent Application Publication
(10) Pub. No.: US 2019/0148004 A1
(43) Pub. Date: May 16, 2019

(54) NON-INVASIVE METHOD FOR ASSESSING THE PRESENCE AND SEVERITY OF ESOPHAGEAL VARICES

(71) Applicants: UNIVERSITE D'ANGERS, Angers (FR); CENTRE HOSPITALIER UNIVERSITAIRE D'ANGERS, Angers (FR); CENTRE HOSPITALIER UNIVERSITAIRE DE NANTES, Nantes Cedex 1 (FR); APHP (ASSISTANCE PUBLIQUE - HOPITAUX DE PARIS), Paris (FR); UNIVERSITE PARIS DIDEROT - PARIS 7, Paris Cedex 13 (FR)

(72) Inventors: Paul CALES, Avrille (FR); Sylvie SACHER-HUVELIN, Orvault (FR); Jean-Paul GALMICHE, Rouen (FR); Dominique VALLA, Bois Colombes (FR)

(21) Appl. No.: 15/564,835

(22) PCT Filed: Apr. 7, 2016
(86) PCT No.: PCT/EP2016/057653

§ 371 Date: Oct. 6, 2017

(30) Foreign Application Priority Data
Apr. 7, 2015 (EP) 15162685.0
Mar. 30, 2016 (EP) 16163029.8

(51) Int. Cl. G16H 30/40; G01N 33/50

(52) U.S. Cl. CPC G16H 30/40; G01N 2800/085; G01N 33/5091

(57) ABSTRACT

Disclosed is a non-invasive method for assessing the presence and/or severity of varices selected from gastric and esophageal varices in a liver disease patient, wherein the method includes: (a) carrying out one or more non-invasive test(s) for assessing the severity of a hepatic lesion or disorder, wherein the non-invasive test(s) each result in a value; and (b) comparing the value(s) obtained at step (a) with cut-offs of the non-invasive test(s) for assessing the presence and/or severity of varices selected from gastric and esophageal varices.

### FIELD OF INVENTION

The present invention relates to the assessment of the presence and/or severity of varices, including esophageal varices and gastric varices, in particular to the detection of large esophageal varices. More specifically, the present invention relates to a non-invasive method comprising measuring blood markers and/or obtaining physical data and optionally recovering data from an endoscopic capsule for assessing the presence and/or severity of esophageal or gastric varices.

### BACKGROUND OF INVENTION

The majority of patients who succumb to fibrosis or cirrhosis die due to complications of increased portal venous pressure, including variceal hemorrhage, ascites, hepatic encephalopathy, and the like. Indeed, severe fibrosis, especially cirrhosis, induces portal hypertension which, above a portal pressure level of 10 mmHg, provokes esophageal varices. Bleeding from ruptured esophageal varices is a major cause of mortality and economic burden in cirrhosis.

Primary prevention of first bleeding in large esophageal varices significantly reduces mortality. Therefore, the recommended work-up of cirrhotic patients includes systematic screening of large esophageal varices.

The gold standard method to diagnose large esophageal varices is upper gastro-intestinal endoscopy (UGIE). However, UGIE is somewhat limited by some constraints and notably the poor acceptance by the patients, due to the invasiveness of this method.

There is thus a need for non-invasive methods for diagnosing large esophageal varices.

Non-invasive diagnosis of large esophageal varices is currently not accurate enough to be adopted in practice. In particular, esophageal capsule endoscopy (ECE) presents a clinically significant probability of missed esophageal varices (i.e. false negative results) or of false positive results.

There is thus a need for a non-invasive method for diagnosing esophageal varices, which is more accurate than esophageal capsule endoscopy, and in particular which allows reducing missed esophageal varices.

Non-invasive diagnosis of liver fibrosis has gained considerable attention over the last 10 years as an alternative to liver biopsy. The first generation of simple blood fibrosis tests combined common indirect blood markers into a simple ratio, like APRI (Wai et al., Hepatology 2003) or FIB-4 (Sterling et al., Hepatology 2006). The second generation of calculated tests combine indirect and/or direct fibrosis markers by logistic regression, leading to a score, like Fibrotest (Imbert-Bismut et al., Lancet 2001), ELF score (Rosenberg et al., Gastroenterology 2004), FibroMeter™ (Cales et al., Hepatology 2005), Fibrospect™ (Patel et al., J Hepatology 2004), and Hepascore (Adams et al., Clin Chem 2005). For example, WO2005/116901 describes a non-invasive method for assessing the presence of a liver disease and its severity, by measuring levels of specific variables, including biological variables and clinical variables, and combining said variables into mathematical functions, generally binary mathematical function to provide a score result, often called “score of fibrosis”.

There is currently a need for a non-invasive diagnostic test for directly assessing the presence and/or severity of varices.

WO2014/190170 describes a non-invasive test for assessing hepatic vein pressure gradient (HVPG) in cirrhotic patients, and suggests that this test may be used for assessing the absence of varices. However, the non-invasive test of WO2014/190170 presents the drawback of using blood markers without clinical potential, because these markers, while commonly used for research purpose, may not easily be used for clinical diagnosis, due either to a difficult implementation or to the cost of the measurement. Moreover, there is no experimental demonstration in WO2014/190170 that the described non-invasive test may efficiently be used for assessing the presence of esophageal varices. Furthermore, the non-invasive test of WO2014/190170 only results in two situations: either the patient shows a HVPG lower than 12 mmHg, and is diagnosed as not presenting esophageal varices, either the patient shows a HVPG of at least 12 mmHg, and an additional test is required for assessing the presence of esophageal varices (usually endoscopy).

The non-invasive test of WO2014/190170 was developed on cirrhotic patients, i.e. in patients already diagnosed with cirrhosis. However, the construction and performance evaluation of non-invasive tests of cirrhosis are limited by the characteristics of liver biopsy which is an imperfect gold standard. Therefore, a non-invasive test for assessing the presence of esophageal varices should ideally circumvent the intermediate step of cirrhosis diagnosis.

There is thus a need for a non-invasive method for diagnosing esophageal varices without the drawbacks of the non-invasive tests of the prior art.

In the present invention, the Applicants develop a non-invasive method for diagnosing esophageal varices in a patient with a liver disease (whether or not this patient was previously diagnosed as cirrhotic), wherein said method comprises performing a non-invasive diagnostic test for assessing the severity of a hepatic condition, using cut-offs for assessing the presence of varices instead of cut-offs for assessing the severity of a hepatic condition. In one embodiment, the method of the invention further comprises combining in a score blood markers, clinical markers, and data obtained by esophageal capsule endoscopy.

Experimental data obtained by the Applicant demonstrate that the non-invasive method of the invention may be used in any liver disease patient, and strongly reduces the number of missed esophageal varices as compared to ECE for example.

### SUMMARY

The present invention thus relates to a non-invasive method for assessing the presence and/or severity of varices, selected from gastric and esophageal varices in a liver disease patient, wherein said method comprises:
(a) carrying out a non-invasive test for assessing the severity of a hepatic lesion or disorder, wherein said non-invasive test results in a value, and
(b) comparing the value obtained at step (a) with cut-offs of said non-invasive test for assessing the presence and/or severity of varices, selected from gastric and esophageal varices.

In one embodiment, the present invention relates to a non-invasive method for assessing the presence and/or severity of varices, selected from gastric and esophageal varices in a liver disease patient, wherein said method comprises:
(a) carrying out at least one non-invasive test for assessing the severity of a hepatic lesion or disorder selected from the group comprising ELF, FibroSpect™, APRI, FIB-4, Hepascore, FibroMeter™, CirrhoMeter™, CombiMeter, Elasto-FibroMeter™, Elasto-Fibrotest, and InflaMeter™, and optionally measuring the platelet count in a blood sample from said patient, wherein said non-invasive test and optionally said platelet count results in at least one value, and
(b) comparing the at least one value obtained at step (a) with cut-offs of said non-invasive test for assessing the presence and/or severity of varices, selected from gastric and esophageal varices.

The present invention also relates to a non-invasive method for assessing the presence and/or severity of varices, selected from gastric and esophageal varices in a liver disease patient, wherein said method comprises:
(a) carrying out one or more non-invasive test(s) for assessing the severity of a hepatic lesion or disorder, wherein said non-invasive test(s) each result in a value, and
(b) comparing the value(s) obtained at step (a) with cut-offs of said non-invasive test(s) for assessing the presence and/or severity of varices, selected from gastric and esophageal varices.

In one embodiment, the preset invention relates to a non-invasive method, wherein step a) comprises carrying out at least one non-invasive test for assessing the severity of a hepatic lesion or disorder selected from the group comprising ELF, FibroSpect™, APRI, FIB-4, Hepascore, FibroMeter™, CirrhoMeter™, CombiMeter, Elasto-FibroMeter™, Elasto-Fibrotest, and InflaMeter™; and carrying out another non-invasive test for assessing the severity of a hepatic lesion or disorder selected from the group comprising ELF, FibroSpect™, APRI, FIB-4, Hepascore, FibroMeter™, CirrhoMeter™, CombiMeter, Elasto-FibroMeter™, Elasto-Fibrotest, InflaMeter™, VCTE (also known as Fibroscan), ARFI, VTE, supersonic elastometry and MRI stiffness, and optionally measuring the platelet count in a blood sample from said patient, wherein the at least two non-invasive tests are different.

In one embodiment, the method is for assessing the presence of large esophageal varices.

In one embodiment, said cut-offs are a negative predictive value (NPV) cut-off and a positive predictive value (PPV) cut-off, or a sensitivity cut-off and a specificity cut-off.

In one embodiment, said NPV and PPV cut-offs define two predictive zones, a NPV predictive zone and a PPV predictive zone.

In one embodiment,
a value obtained in step (a) below the NPV cut-off or below the sensitivity cut-off is indicative of the absence of varices, selected from gastric and esophageal varices, preferably of large esophageal varices, in the patient, and
a value obtained in step (a) above the PPV cut-off or above the specificity cut-off is indicative of the presence of varices, selected from gastric and esophageal varices, preferably of large esophageal varices, in the patient.

In one embodiment,
one or more value obtained in step (a) below the NPV cut-off or below the sensitivity cut-off is in the NPV predictive zone and is indicative of the absence of varices, selected from gastric and esophageal varices, preferably of large esophageal varices, in the patient, and
one or more value obtained in step (a) above the PPV cut-off or above the specificity cut-off is in the PPV predictive zone and is indicative of the presence of varices, selected from gastric and esophageal varices, preferably of large esophageal varices, in the patient.

In one embodiment, if the value obtained in step (a) is in the indeterminate zone between the NPV cut-off and the PPV cut-off or between the sensitivity cut-off and the specificity cut-off, then the method further comprises one or more repetition of step (a) and step (b) wherein at least one non-invasive test carried out for assessing the severity of a hepatic lesion or disorder is different from the at least one non-invasive test previously carried out, thereby defining new NPV and PPV predictive zones and assessing the presence and/or severity of varices in said patient through the use of multiple NPV and PPV predictive zones.

In one embodiment, if the value obtained in step (a) is in the indeterminate zone between the NPV cut-off and the PPV cut-off or between the sensitivity cut-off and the specificity cut-off, then the method further comprises the steps of:
(c) measuring at least one of the following variables from the subject:
   biomarkers,
   clinical data,
   binary markers,
   physical data from medical imaging or clinical measurement,
(d) obtaining imaging data on varices status, wherein said imaging data are obtained by a non-invasive imaging method,
(e) mathematically combining, preferably in a binary logistic regression,
   the variables obtained in step (c), or any mathematical combination thereof with,
   the data obtained at step (d),
wherein the mathematical combination results in a diagnostic score, and
(f) assessing the presence and/or severity of varices, selected from gastric and esophageal varices, preferably of large esophageal varices, based on the diagnostic score obtained in step (e).

In one embodiment, the imaging data on varices status are obtained by a non-invasive imaging method, preferably esophageal capsule endoscopy; or by a radiologic method, preferably a scanner.

In one embodiment, the non-invasive test carried out in step (a) is a blood test, preferably selected from ELF, FibroSpect™, APRI, FIB-4, Hepascore, Fibrotest™, FibroMeter™, CirrhoMeter™, CombiMeter, Elasto-FibroMeter™, Elasto-Fibrotest, InflaMeter™; or a physical method, preferably selected from VCTE, ARFI, VTE, supersonic elastometry or MRI stiffness.

In one embodiment, at step (c), the obtained variables are the variables of the non-invasive test carried out in step (a).

In one embodiment, the non-invasive test carried out in step (a) is a CirrhoMeter.

In one embodiment, the non-invasive method of the invention comprises carrying out at least two non-invasive tests for assessing the severity of a hepatic lesion or disorder, wherein said at least two non-invasive tests are different.

In one embodiment, the non-invasive test carried out in step (a) is a CirrhoMeter, and wherein the variables obtained at step (c) are the variables of a CirrhoMeter.

In one embodiment, the patient is affected with a chronic hepatic disease, preferably selected from the group comprising chronic viral hepatitis C, chronic viral hepatitis B, chronic viral hepatitis D, chronic viral hepatitis E, non-alcoholic fatty liver disease (NAFLD), alcoholic chronic liver disease, autoimmune hepatitis, primary biliary cirrhosis, hemochromatosis and Wilson disease.

In one embodiment, the patient is a cirrhotic patient.

Another object of the invention is a non-invasive method for assessing the presence and/or severity of varices, selected from gastric and esophageal varices, preferably of large esophageal varices, in a hepatic disease patient, wherein said method comprises:
i. measuring at least one of the following variables from the subject:
   biomarkers,
   clinical data,
   binary markers,
   physical data from medical imaging or clinical measurement,
ii. obtaining imaging data on varices status, wherein said imaging data are obtained by a non-invasive imaging method,
iii. mathematically combining, preferably in a binary logistic regression,
   the variables obtained in step (i), or any mathematical combination thereof with
   the data obtained at step (ii),
   wherein the mathematical combination results in a diagnostic score, and
iv. assessing the presence and/or severity of varices, selected from gastric and esophageal varices, preferably of large esophageal varices, based on the diagnostic score obtained in step (iii).

In one embodiment, the patient was previously diagnosed as cirrhotic, or wherein the patient previously obtained a value between the NPV and the PPV cut-offs in a method as described hereinabove.

The present invention also relates to a microprocessor comprising a computer algorithm carrying out the method as described hereinabove.

### Definitions

In the present invention, the following terms have the following meanings:
In the present invention, the indefinite article “a” preceding an object (e.g. a non-invasive test) refers to one or more of said object (e.g. one or more non-invasive test(s)).
“Algorithm” refers to the combination, simultaneously or sequentially, of at least two non-invasive tests into a decision tree for assessing the severity of a hepatic lesion or disorder in the method of the invention.
“Positive predictive value (PPV)” refers to the proportion of patients with a positive test that actually have disease; if 9 of 10 positive test results are correct (true positive), the PPV is 90%. Because all positive test results have some number of true positives and some false positives, the PPV describes how likely it is that a positive test result in a given patient population represents a true positive.
“Negative predictive value (NPV)” refers to the proportion of patients with a negative test result that are actually disease free; if 8 of 10 negative test results are correct (true negative), the NPV is 80%. Because not all negative test results are true negatives, some patients with a negative test result actually have the disease. The NPV describes how likely it is that a negative test result in a given patient population represents a true negative.
“AUROC” stands for area under the ROC curve, and is an indicator of the accuracy of a diagnostic test. In statistics, a receiver operating characteristic (ROC), or ROC curve, is a graphical plot that illustrates the performance of a binary classifier system as its discrimination threshold is varied. The curve is created by plotting the sensitivity against the specificity (usually 1—specificity) at successive values from 0 to 1. ROC curve and AUROC are well-known in the field of statistics.
“Sensitivity” (also called true positive rate) measures the proportion of actual positives which are correctly identified as such.
“Specificity” (also called true negative rate) measures the proportion of negatives which are correctly identified as such.
“Esophageal varices” refers to dilated sub-mucosal veins in the lower third of the esophagus. Esophageal varices are a consequence of portal hypertension (referring to portal pressure of at least about 10 mm Hg, preferably at least about 12 mm Hg), commonly due to cirrhosis. As used herein, the term “large esophageal varices” may refer to varices of at least about 5 mm in diameter, such as, for example, when measured by UGIE. The term “large esophageal varices” may also refer to esophageal varices of at least 15% of the esophageal circumference, preferably of at least 25, 30, 40, 50% or more.
“Gastric varices” refers to dilated sub-mucosal veins in the stomach. Gastric varices are a consequence of portal hypertension (referring to portal pressure of at least about 10 mm Hg, preferably at least about 12 mm Hg), commonly due to cirrhosis.
“About” preceding a figure means plus or less 10% of the value of said figure.
“Biomarker” refers to a variable that may be measured in a sample from the subject, wherein the sample may be a bodily fluid sample, such as, for example, a blood, serum or urine sample, preferably a blood or serum sample.
“Clinical data” refers to a data recovered from external observation of the subject, without the use of laboratory tests and the like.
“Binary marker” refers to a marker having the value 0 or 1 (or yes or no).
“Physical data” refers to a variable obtained by a physical method.
“Blood test” corresponds to a test comprising non-invasively measuring at least one data, and, when at least two data are measured, mathematically combining said at least two data within a score. In the present invention, said data may be a biomarker, a clinical data, a physical data, a binary marker or any combination thereof (such as, for example, any mathematical combination within a score).
“Score” refers to any digit value obtained by the mathematical combination (univariate or multivariate) of at least one biomarker and/or at least one clinical data and/or at least one physical data and/or at least one binary marker and/or at least one blood test result. In one embodiment, a score is an unbound digit value. In another embodiment, a score is a bound digit value, obtained by a mathematical function. Preferably, a score ranges from 0 to 1. In one embodiment, the at least one biomarker and/or at least one clinical data and/or at least one physical data and/or at least one binary marker and/or at least one score, mathematically combined in a score are independent, i.e. give each an information that is different and not linked to the information given by the others.
“Patient” refers to a subject awaiting the receipt of, or is receiving medical care or is/will be the object of a medical procedure for treating a hepatic disease.

### DETAILED DESCRIPTION

The present invention relates to non-invasive methods for assessing the presence and/or severity of varices, selected from esophageal varices and gastric varices in a liver disease patient, preferably is a patient with chronic liver disease.

In one embodiment, the method of the invention is an in vitro method.

In one embodiment, the method of the invention is for assessing the presence of esophageal varices, preferably of esophageal varices of at least about 1 mm in diameter, preferably of at least about 1.5, 2, 2.5, 3, 3.5, 4, 4.5, 5, 6, 7, 8, 9, 10, 15, or 20 mm or more, such as, for example, when measured by UGIE. In one embodiment, the method of the invention is for assessing the presence of large esophageal varices, i.e. of esophageal varices of at least 15% of the esophageal circumference, preferably of at least 25, 30, 40, 50% or more when measured by ECE, or varices of at least about 5 mm in diameter, such as, for example, when measured by UGIE.

In another embodiment, the method of the invention is for assessing the presence of gastric varices such as, for example, gastro-esophageal varices or preferably isolated gastric varices, usually fundal varices.

The present invention first relates to a non-invasive method for assessing the presence and/or severity of varices, selected from esophageal varices and gastric varices in a liver disease patient, using a non-invasive test for assessing the severity of a hepatic lesion or disorder.

The present invention also relates to a non-invasive method for assessing the presence and/or severity of varices, selected from esophageal varices and gastric varices in a liver disease patient, using one or more non-invasive tests for assessing the severity of a hepatic lesion or disorder. The present invention relates to a non-invasive method for assessing the presence and/or severity of varices, selected from esophageal varices and gastric varices in a liver disease patient, using at least two, at least three, at least four or more non-invasive tests for assessing the severity of a hepatic lesion or disorder. In one embodiment, the present invention relates to a non-invasive method for assessing the presence and/or severity of varices, selected from esophageal varices and gastric varices in a liver disease patient, using two, three, four, five or more non-invasive tests for assessing the severity of a hepatic lesion or disorder.

However, in the usual test(s) for assessing the severity of a hepatic lesion or disorder, the cut-offs of said test(s) for assessing the presence and/or severity of varices are determined, preferably as described hereinabove.

Hence, this invention relates to a method comprising:
(a) carrying out the non-invasive test, wherein said non-invasive test results in a value, and
(b) comparing the value obtained at step (a) with said cut-offs of said test for assessing the presence and/or severity of varices.

This invention also relates to a method comprising:
(a) carrying out one or more non-invasive tests, wherein said non-invasive tests each result in a value, and
(b) comparing the values obtained at step (a) with said cut-offs of said tests for assessing the presence and/or severity of varices.

This invention also relates to a method comprising:
(a) carrying out at least one non-invasive tests, wherein said non-invasive tests results in a value, and
(b) comparing the at least one value obtained at step (a) with said cut-offs of said tests for assessing the presence and/or severity of varices.

In one embodiment, the invention relates to a method comprising carrying out at least two non-invasive tests, wherein said non-invasive tests each result in a value, said values being compared with cut-offs of said tests for assessing the presence and/or severity of varices.

In another embodiment, the invention relates to a method comprising carrying out at least three non-invasive tests, wherein said non-invasive tests each result in a value, said values being compared with cut-offs of said tests for assessing the presence and/or severity of varices.

In another embodiment, the invention relates to a method comprising carrying out at least four non-invasive tests, wherein said non-invasive tests each result in a value, said values being compared with cut-offs of said tests for assessing the presence and/or severity of varices.

In one embodiment, the invention relates to a method comprising carrying out simultaneously two non-invasive tests, wherein said non-invasive tests each result in a value, said values being compared with cut-offs of said tests for assessing the presence and/or severity of varices.

In one embodiment, the invention relates to a method comprising carrying out sequentially two non-invasive tests, wherein said non-invasive tests each result in a value, said values being compared with cut-offs of said tests for assessing the presence and/or severity of varices. In another embodiment, the invention relates to a method comprising carrying out sequentially three or more non-invasive tests, wherein said non-invasive tests each result in a value, said values being compared with cut-offs of said tests for assessing the presence and/or severity of varices.

Hence in one embodiment, the invention relates to a method comprising carrying out two non-invasive tests in an algorithm, wherein said non-invasive tests each result in a value, said values being compared with cut-offs of said tests for assessing the presence and/or severity of varices. In another embodiment, the invention relates to a method comprising carrying out three, or four, or five, or more non-invasive tests in an algorithm, wherein said non-invasive tests each result in a value, said values being compared with cut-offs of said tests for assessing the presence and/or severity of varices.

In one embodiment, the two, three, four, five or more non-invasive tests carried out in the method of the invention are different. Hence in one embodiment, the two, three, four, five or more non-invasive tests carried out in the method of the invention are not repetitions of the same non-invasive tests.

In one embodiment, the method of the invention further comprises a first step of determining the cut-offs of said test(s) for assessing the presence and/or severity of varices, using a population of reference.

Two cut-offs may usually be determined for diagnostic tests, i.e. the NPV cut-off and the PPV cut-off. A value below the NPV cut-off is indicative of the absence of the diagnostic target, whereas a value above the PPV cut-off is indicative of the presence of the diagnostic target. Between the NPV cut-off and the PPV cut-off is an indeterminate zone, wherein no conclusion may be raised regarding the presence or absence of the diagnosis target.

Hence the cut-offs determined for a diagnostic test, the NPV and PPV cut-offs determine two predictive zones: the NPV predictive zone below the NPV cut-off, and the PPV predictive zone above the PPV cut-off. The zone between the NPV and PPV cut-offs is referred to as the indeterminate zone.

In one embodiment, the method of the invention comprises carrying out one non-invasive test for assessing the severity of a hepatic lesion or disorder in step a). Said one non-invasive test is associated with two cut-offs. In one embodiment said cut-offs are NPV and PPV cut-offs thereby defining a NPV predictive zone below the NPV cut-off, and a PPV predictive zone above the PPV cut-off.

In another embodiment, step a) of the method of the invention comprises carrying out two non-invasive tests for assessing the severity of a hepatic lesion or disorder in an algorithm. Said two non-invasive test, for example non-invasive test x and non-invasive test y, are each associated with two cut-offs. In one embodiment each non-invasive test is associated with a NPV and a PPV cut-offs, said NPV (for example NPVₓ and NPV_{y}) and PPV (for example PPVₓ and PPV_{y}) cut-offs defining the predictive zones of the algorithm. In one embodiment, the NPV predictive zone is below at least one of the two NPV cut-offs (below NPVₓ or NPV_{y}) and the PPV predictive zone is above the two PPV cut-offs (above PPVₓ and PPV_{y}). In another embodiment, the NPV predictive zone is below the two cut offs (below NPVₓ and NPV_{y}), and the PPV predictive zone is above the two PPV cut-offs (above PPVₓ and PPV_{y}).

In one embodiment, the method of the invention allows the assessment of the presence and/or severity of varices through the use of single predictive zones, i.e. through the use of one NPV and one PPV predictive zone.

In one embodiment, the method of the invention further comprises, in particular for patients classified in the indeterminate zone between the NPV and PPV cut-offs, one or more repetition of step (a) and step (b) wherein at least one non-invasive test carried out for assessing the severity of a hepatic lesion or disorder is different from the at least one non-invasive test previously carried out. In another embodiment, the method of the invention further comprises, in particular for patients classified in the indeterminate zone, one or more repetition of step (a) and step (b) wherein the algorithm carried out for assessing the severity of a hepatic lesion or disorder is different from the algorithm previously carried out.

Hence in one embodiment, the NPV and PPV cut-offs determined for the at least one non-invasive test carried out in the repeated step a) define new NPV and PPV predictive zones. In another embodiment, the sets of NPV and PPV cut-offs determined for the at least one non-invasive test carried out in the second step a) and for the at least one non-invasive test carried out in any subsequent step a) each define new NPV and PPV predictive zones. In one embodiment, the method of the invention allows the assessment of the presence and/or severity of varices through the use of multiple predictive zones.

In one embodiment, the method of the invention further comprises a first step of determining the cut-offs of said test(s) for assessing the presence and/or severity of varices, and the associated predictive zones using a population of reference. In another embodiment, the method of the invention further comprises a first step of determining the cut-offs of said test(s) for assessing the presence and/or severity of varices carried out in one or more repetition of step a), and the associated multiple predictive zones using a population of reference.

According to one embodiment, to determine multiple predictive zones in a population of reference, the NPV and PPV predictive zones are first determined using the two non-invasive tests having the largest predictive zones. The choice of the two tests can be done according to several classical statistical techniques, for example the most accurate tests according to multivariate analysis or correlation. The NPV and PPV predictive zones are determined as described hereinabove, using the NPV and PPV cut-offs of each of the two non-invasive tests. Then, a new population of reference is obtained by excluding the patients of the original population of reference located in the NPV and PPV predictive zones. Subsequently new NPV and PPV predictive zones are determined on the smaller population of reference using a different set of two non-invasive tests. At least one of the two non-invasive tests must be different from those used in the first set. Otherwise, the NPV and PPV zones will be empty since the patients within a NPV and PPV zone thus determined have already been excluded. Thus, using the NPV and PPV cut-offs of the new set of two non-invasive tests, new NPV and PPV predictive zones are determined. The process can be reiterated on a new smaller population of reference by excluding the patients located in the second NPV and PPV predictive zones.

In one embodiment, the method of the invention comprises one or more repetition of step a) and step b), wherein at least one non-invasive test carried out for assessing the severity of a hepatic lesion or disorder is different from the at least one non-invasive test previously carried out.

In another embodiment, the method of the invention comprises two or more repetitions of step a) and step b), wherein for each repetition, at least one non-invasive test carried out for assessing the severity of a hepatic lesion or disorder is different from the at least one non-invasive test previously carried out.

In another embodiment, the method of the invention comprises three, four, five or more repetitions of step a) and step b), wherein for each repetition, at least one non-invasive test carried out for assessing the severity of a hepatic lesion or disorder is different from the at least one non-invasive test previously carried out.

In one embodiment, the cut-offs are sensitivity cut-offs and specificity cut-offs. A value below the sensitivity cut-off is indicative of the absence of the diagnostic target, whereas a value above the specificity cut-off is indicative of the presence of the diagnostic target. Between the sensitivity cut-off and the specificity cut-off is an indeterminate zone, wherein no conclusion may be raised regarding the presence or absence of the diagnosis target.

In the present invention, the diagnostic target is the presence of varices, selected from gastric and esophageal varices (preferably large esophageal varices), and a value below the NPV cut-off is indicative of the absence of varices, selected from gastric and esophageal varices (preferably large esophageal varices), whereas a value above the PPV cut-off is indicative of the presence of varices, selected from gastric and esophageal varices (preferably large esophageal varices). Between the NPV cut-off and the PPV cut-off is an indeterminate zone, wherein no conclusion may be raised regarding the presence or absence of varices, selected from gastric or esophageal varices (preferably large esophageal varices).

In one embodiment,
one or more value obtained in step (a) below the NPV cut-off or below the sensitivity cut-off is in the NPV predictive zone and is indicative of the absence of varices, selected from gastric and esophageal varices, preferably of large esophageal varices, in the patient, and
one or more value obtained in step (a) above the PPV cut-off or above the specificity cut-off is in the PPV predictive zone and is indicative of the presence of varices, selected from gastric and esophageal varices, preferably of large esophageal varices, in the patient.

In the present invention, the diagnostic target is the presence of varices, selected from gastric and esophageal varices (preferably large esophageal varices), and a value below the sensitivity cut-off is indicative of the absence of varices, selected from gastric and esophageal varices (preferably large esophageal varices), whereas a value above the specificity cut-off is indicative of the presence of varices, selected from gastric and esophageal varices (preferably large esophageal varices). Between the sensitivity cut-off and the specificity cut-off is an indeterminate zone, wherein no conclusion may be raised regarding the presence or absence of varices, selected from gastric and esophageal varices (preferably large esophageal varices).

The skilled artisan knows how to determine cut-offs for a diagnostic target (see for example a method for determining cut-offs of a diagnostic target in Cales, Liver Intern 2008), using a reference population.

In one embodiment, the NPV cut-offs and the PPV cut-offs are determined in a reference population in order to reach:
a NPV of at least about 80%, preferably of at least about 85%, 86%, 87%, 88%, 89%, 90%, 91%, 92%, 93%, 94%, 95%, 96%, 97%, 98%, 99% or more, and/or
a PPV of at least about 80%, preferably of at least about 85%, 86%, 87%, 88%, 89%, 90%, 91%, 92%, 93%, 94%, 95%, 96%, 97%, 98%, 99% or more.

In one embodiment, the NPV cut-offs and the PPV cut-offs are determined in a reference population in order to reach a NPV of at least 95% and a PPV of at least 90%.

In one embodiment, the sensitivity cut-offs and the specificity cut-offs are determined in a reference population in order to reach:
a sensitivity of at least about 80%, preferably of at least about 85%, 86%, 87%, 88%, 89%, 90%, 91%, 92%, 93%, 94%, 95%, 96%, 97%, 98%, 99% or more, and/or
a specificity of at least about 80%, preferably of at least about 85%, 86%, 87%, 88%, 89%, 90%, 91%, 92%, 93%, 94%, 95%, 96%, 97%, 98%, 99% or more.

In one embodiment, the sensitivity cut-offs and the specificity cut-offs are determined in a reference population in order to reach a sensitivity of at least 95% and a specificity of at least 90%.

In one embodiment, the reference population comprises liver disease patients, preferably patients with chronic liver disease, wherein for each patient the value of the non-invasive test was measured and the status regarding varices, selected from gastric and esophageal varices is known, i.e. absence or presence or size of varices, selected from gastric and esophageal varices, preferably of large esophageal varices (i.e. in one embodiment, an upper gastro-intestinal endoscopy was performed).

Therefore, the present invention is based on the application of a diagnostic test constructed for diagnosing the severity of a hepatic lesion or disorder to the diagnostic of another diagnostic target, varices, through the determination of cut-offs specific for esophageal varices diagnostic.

The experimental data provided in the Examples surprisingly demonstrated that the use of cut-offs specific for varices, selected from gastric and esophageal varices diagnostic instead of cut-offs specific for cirrhosis diagnosis increases the accuracy of the diagnostic test for diagnosing varices, selected from gastric and esophageal varices.

For example, CirrhoMeter™ is a non-invasive diagnostic test primarily constructed for diagnosing cirrhosis (i.e. cut-offs specific for cirrhosis were measured). In the present invention, CirrhoMeter™ cut-offs specific for esophageal varices (preferably large esophageal varices) were measured. CirrhoMeter™ cut-offs for cirrhosis or esophageal varices are shown in the table below.

| Diagnostic target | 95% NPV cut-off | 90% PPV cut-off |
|---|---|---|
| Cirrhosis | 0.302 | 0.725 |
| Esophageal varices | 0.545 | 0.9994 |

Therefore, in one embodiment, the method of the invention is for classifying a patient into one of the three following classes:
i. absence of varices, selected from gastric and esophageal varices, preferably large esophageal varices (for patients having a value below the NPV cut-off value or below the sensitivity cut-off value),
ii. presence of varices, selected from gastric and esophageal varices, preferably large esophageal varices (for patients having a value above the PPV cut-off value or above the specificity cut-off value), or
iii. indeterminate zone (for patients having a value ranging between the NPV cut-off value and the PPV cut-off value or between the sensitivity cut-off value and the specificity cut-off value).

In one embodiment, the method of the invention further comprises, in particular for patients classified in the indeterminate zone, one or more repetition of step (a) and step (b) wherein at least one non-invasive test carried out for assessing the severity of a hepatic lesion or disorder is different from the at least one non-invasive test previously carried out, thereby defining new NPV and PPV predictive zones and assessing the presence and/or severity of varices in said patient through the use of multiple NPV and PPV predictive zones.

In one embodiment, the method of the invention further comprises, in particular for patients classified in the indeterminate zone, the following steps:
(c) measuring at least one of the following variables from the subject:
   biomarkers,
   clinical data,
   binary markers,
   physical data from medical imaging or clinical measurement
(d) obtaining imaging data on varices status, wherein said imaging data are obtained by a non-invasive imaging method,
(e) mathematically combining:
   the variables obtained in step (c), or any mathematical combination thereof with
   the data obtained at step (d),
wherein the mathematical combination results in a diagnostic score, and
(f) assessing the presence and/or severity of varices, selected from gastric and esophageal varices (preferably large esophageal varices) based on the diagnostic score obtained in step (e).

In one embodiment, the assessment of the presence and/or severity of step (f) comprises comparing the score obtained in step (e) with cut-off values for the diagnostic test resulting in the diagnostic score of the invention. As explained hereinabove, two cut-offs may be determined for the diagnostic test resulting in the diagnostic score of the invention: the NPV cut-off and the PPV cut-off, or the sensitivity cut-off and the specificity cut-off.

In one embodiment, the NPV cut-offs and the PPV cut-offs are determined in a reference population in order to reach:
a NPV of at least about 75%, preferably of at least about 80%, more preferably of at least about 85%, 86%, 87%, 88%, 89%, 90%, 91%, 92%, 93%, 94%, 95%, 96%, 97%, 98%, 99% or more, and/or
a PPV of at least about 75%, preferably of at least about 80%, preferably of at least about 85%, 86%, 87%, 88%, 89%, 90%, 91%, 92%, 93%, 94%, 95%, 96%, 97%, 98%, 99% or more.

In one embodiment, the NPV cut-offs and the PPV cut-offs are determined in a reference population in order to reach a NPV of at least 95% and a PPV of at least 90%.

In one embodiment, the sensitivity cut-offs and the specificity cut-offs are determined in a reference population in order to reach:
a sensitivity of at least about 75%, preferably of at least about 80%, more preferably of at least about 85%, 86%, 87%, 88%, 89%, 90%, 91%, 92%, 93%, 94%, 95%, 96%, 97%, 98%, 99% or more, and/or
a specificity of at least about 75%, preferably of at least about 80%, preferably of at least about 85%, 86%, 87%, 88%, 89%, 90%, 91%, 92%, 93%, 94%, 95%, 96%, 97%, 98%, 99% or more.

In one embodiment, the sensitivity cut-offs and the specificity cut-offs are determined in a reference population in order to reach a sensitivity of at least 95% and a PPV of at least 90%.

In the present invention, the diagnostic target is the presence of varices, selected from gastric and esophageal varices (preferably large esophageal varices), and a diagnostic score below the NPV (or sensitivity) cut-off is indicative of the absence of varices, selected from gastric and esophageal varices (preferably large esophageal varices), whereas a diagnostic score above the PPV (or specificity) cut-off is indicative of the presence of varices, selected from gastric and esophageal varices (preferably large esophageal varices). Between the NPV (or sensitivity) cut-off and the PPV (or specificity) cut-off is an indeterminate zone, wherein no conclusion may be raised regarding the presence or absence of varices, selected from gastric and esophageal varices (preferably large esophageal varices).

Therefore, in one embodiment, the method of the invention is for classifying a patient into one of the three following classes:
i. absence of varices, selected from gastric and esophageal varices, preferably absence of large esophageal varices (for patients having a diagnostic score below the NPV (or sensitivity) cut-off value),
ii. presence of varices, selected from gastric and esophageal varices, preferably presence of large esophageal varices (for patients having a diagnostic score above the PPV (or specificity) cut-off value), or
iii. indeterminate zone (for patients having a diagnostic score ranging between the NPV cut-off value and the PPV cut-off value or between the sensitivity and specificity cut-off values).

In one embodiment, patients having a diagnostic score between the NPV and PPV cut-offs required an invasive test for determining the presence or absence of varices, selected from gastric and esophageal varices, such as, for example, endoscopy (UGIE).

In one embodiment, patients having a diagnostic score between the sensitivity and specificity cut-offs required an invasive test for determining the presence or absence of varices, selected from gastric and esophageal varices, such as, for example, endoscopy (UGIE).

In one embodiment, in step (c), the obtained variables are the variables of the non-invasive test carried out in step (a).

In one embodiment, at step (e), the variables obtained at step (c) are mathematically combined in a non-invasive test value, preferably in a score, prior to the mathematical combination with the data obtained at step (d).

In one embodiment, the present invention thus relates to a non-invasive method for assessing the presence and/or severity of varices, selected from gastric and esophageal varices (preferably of large esophageal varices) in a liver disease patient, preferably in a patient with chronic liver disease, wherein said method comprises:
(a) carrying out a non-invasive test for assessing the severity of a hepatic lesion or disorder, wherein said non-invasive test results in a value, and
(b) comparing the value obtained at step (a) with cut-offs of said non-invasive test for assessing the presence and/or severity of varices, selected from gastric and esophageal varices (preferably large esophageal varices), thereby determining if the patient does not present varices, selected from gastric and esophageal varices, presents varices, selected from gastric and esophageal varices or is in an indeterminate zone, and
for patients in the indeterminate zone, the method of the invention further comprises:
(c) measuring at least one of the following variables from the subject:
   biomarkers,
   clinical data,
   binary markers,
   physical data from medical imaging or clinical measurement
(d) obtaining imaging data on varices status, wherein said imaging data are obtained by a non-invasive imaging method,
(e) mathematically combining
   the variables obtained in step (c), or any mathematical combination thereof with
   the data obtained at step (d),
wherein the mathematical combination results in a diagnostic score, and
(f) assessing the presence and/or severity of varices, selected from gastric and esophageal varices (preferably large esophageal varices) based on the diagnostic score obtained in step (e).

An algorithm corresponding to the non-invasive diagnostic method of the invention is shown in FIG. 6.

In one embodiment, the determination of cut-offs for gastric varices is performed in the same way as for large esophageal varices (as illustrated in the Examples): first those of non-invasive test and then those of a score combining non-invasive test and ECE.

In one embodiment, the non-invasive test for assessing the severity of a hepatic lesion or disorder is a biomarker, a clinical data, a binary marker, a blood test or a physical method.

In one embodiment, the non-invasive test results in a value, preferably in a score.

In one embodiment, the non-invasive test is selected from the group comprising age, spleen diameter, ALT, leucocytes, body mass index, GGT, alpha2-macroglobulin, weight, segmented leucocytes, height, monocytes, hemoglobin, P2/MS score, alpha-fetoprotein, alkaline phosphatases, sodium, platelets, AST, InflaMeter, creatinine, urea, APRI, Child-Pugh score, FIB-4, VCTE, albumin, FibroMeter (such as, for example, FibroMeter for cause, FibroMeter^{V2G} or FibroMeter^{V3G}), prothrombin index, CirrhoMeter (such as, for example, CirrhoMeter^{V2G} or CirrhoMeter^{VV3G}), bilirubin, Elasto-FibroMeter (such as, for example, Elasto-FibroMeter^{V2G}), hyaluronate, QuantiMeter (such as, for example, QuantiMeter for cause or QuantiMeter^{V2G}), Hepascore, Fibrotest, Fibrospect, Elasto-Fibrotest, ELF score and any mathematical combination thereof, such as, for example, AST/ALT, AST/ALT+prothrombin, AST/ALT+hyaluronate.

In another embodiment, the at least one non-invasive test carried out in step (a) is selected from platelets, ELF, FibroSpect™, APRI, FIB-4, Hepascore, Fibrotest™, FibroMeter™, CirrhoMeter™, CombiMeter, Elasto-FibroMeter™, Elasto-Fibrotest, InflaMeter™; VCTE, ARFI, VTE, supersonic elastometry and/or MRI stiffness.

In another embodiment, the at least one non-invasive test carried out in step (a) is selected from ELF, FibroSpect™, APRI, FIB-4, Hepascore, Fibrotest™, FibroMeter™, CirrhoMeter™, CombiMeter, Elasto-FibroMeter™, Elasto-Fibrotest, InflaMeter™; VCTE, ARFI, VTE, supersonic elastometry and/or MRI stiffness.

In another embodiment, the at least one non-invasive test carried out in step (a) is selected from ELF, FibroSpect™, APRI, FIB-4, Hepascore, FibroMeter™, CirrhoMeter™, CombiMeter, Elasto-FibroMeter™, Elasto-Fibrotest, InflaMeter™; VCTE, ARFI, VTE, supersonic elastometry and/or MRI stiffness.

In another embodiment, the at least one non-invasive test carried out in step (a) is selected from ELF, FibroSpect™, APRI, FIB-4, Hepascore, FibroMeter™, CirrhoMeter™, CombiMeter, Elasto-FibroMeter™, Elasto-Fibrotest, InflaMeter™, and/or VCTE (also known as Fibroscan).

In another embodiment, the at least one non-invasive test carried out in step (a) is selected from ELF, FibroSpect™, APRI, FIB-4, Hepascore, FibroMeter™, CirrhoMeter™, CombiMeter, Elasto-FibroMeter™, Elasto-Fibrotest, and/or InflaMeter™.

In another embodiment, the at least one non-invasive test carried out in step (a) is selected from FibroMeter™, CirrhoMeter™, CombiMeter, Elasto-FibroMeter™, InflaMeter™, and/or VCTE (also known as Fibroscan).

Preferably, the at least one non-invasive test carried out in step (a) is selected from FibroMeter™, CirrhoMeter™, CombiMeter, Elasto-FibroMeter™, and/or InflaMeter™.

In one embodiment, the method of the invention does not comprise carrying out a Fibrotest™.

Examples of biomarkers include, but are not limited to, glycemia, total cholesterol, HDL cholesterol (HDL), LDL cholesterol (LDL), AST (aspartate aminotransferase), ALT (alanine aminotransferase), ferritin, platelets (PLT), prothrombin time (PT) or prothrombin index (PI) or INR (International Normalized Ratio), hyaluronic acid (HA or hyaluronate), haemoglobin, triglycerides, alpha-2 macroglobulin (A2M), gamma-glutamyl transpeptidase (GGT), urea, bilirubin (such as, for example, total bilirubin), apolipoprotein A1 (ApoA1), type III procollagen N-terminal propeptide (P3NP or P3P), gamma-globulins (GBL), sodium (Na), albumin (ALB) (such as, for example, serum albumin), ferritine (Fer), glucose (Glu), alkaline phosphatases (ALP), YKL-40 (human cartilage glycoprotein 39), tissue inhibitor of matrix metalloproteinase 1 (TIMP-1), TGF, cytokeratine 18 and matrix metalloproteinase 2 (MMP-2) to 9 (MMP-9), haptoglobin, alpha-fetoprotein, creatinine, leukocytes, neutrophils, segmented leukocytes, segmented neutrophils, monocytes, ratios and mathematical combinations thereof, such as, for example AST/ALT (ratio), AST.ALT (product), AST/PLT (ratio), AST/ALT+prothrombin, AST/ALT+hyaluronate.

In one embodiment, step (a) of the non-invasive method of the invention comprises measuring platelets (PLT).

In one embodiment, step (a) of the non-invasive method of the invention comprises carrying out at least one non-invasive test for assessing the severity of a hepatic lesion or disorder and optionally measuring the platelet count in a blood sample from said patient, wherein said at least one non-invasive test and optionally said platelet count result in at least one value.

In one embodiment, step (a) of the non-invasive method of the invention comprises carrying out at least one non-invasive test for assessing the severity of a hepatic lesion or disorder and measuring the platelet count in a blood sample from said patient, wherein said at least one non-invasive test and said platelet count each result in at least one value.

The measurements carried out in the method of the invention are measurements aimed either at quantifying the biomarker (such as, for example, in the case of A2M, HA, bilirubin, PLT, PT, urea, NA, glycemia, triglycerides, ALB or P3P), or at quantifying the enzymatic activity of the biomarker (such as, for example, in the case of GGT, ASAT, ALAT, ALP). Those skilled in the art are aware of various direct or indirect methods for quantifying a given substance or a protein or its enzymatic activity. These methods may use one or more monoclonal or polyclonal antibodies that recognize said protein in immunoassay techniques (such as, for example, radioimmunoassay or RIA, ELISA assays, Western blot, etc.), the analysis of the amounts of mRNA for said protein using techniques of the Northern blot, slot blot or PCR type, techniques such as an HPLC optionally combined with mass spectrometry, etc. The abovementioned protein activity assays use assays carried out on at least one substrate specific for each of these proteins. International patent application WO 03/073822 lists methods that can be used to quantify alpha2 macroglobulin (A2M) and hyaluronic acid (HA or hyaluronate).

By way of examples, and in a non-exhaustive manner, a preferred list of commercial kits or assays that can be used for the measurements of biomarkers carried out in the method of the invention, on blood samples, is given hereinafter:
prothrombin time: the Quick time (QT) is determined by adding calcium thromboplastin (for example, Neoplastin CI plus, Diagnostica Stago, Asnieres, France) to the plasma and the clotting time is measured in seconds. To obtain the prothrombin time (PT), a calibration straight line is plotted from various dilutions of a pool of normal plasmas estimated at 100%. The results obtained for the plasmas of patients are expressed as a percentage relative to the pool of normal plasmas. The upper value of the PT is not limited and may exceed 100%.
A2M: the assaying thereof is carried out by laser immunonephelometry using, for example, a Behring nephelometer analyzer. The reagent may be a rabbit antiserum against human A2M.
HA: the serum concentrations are determined with an ELISA (for example: Corgenix, Inc. Biogenic SA 34130 Mauguio France) that uses specific HA-binding proteins isolated from bovine cartilage.
P3P: the serum concentrations are determined with an RIA (for example: RIA-gnost PIIIP kit, Hoechst, Tokyo, Japan) using a murine monoclonal antibody directed against bovine skin PIIINP.
PLT: blood samples are collected in vacutainers containing EDTA (ethylenediaminetetraacetic acid) (for example, Becton Dickinson, France) and can be analyzed on an Advia 120 counter (Bayer Diagnostic).
Urea: assaying, for example, by means of a “Kinectic UV assay for urea” (Roche Diagnostics).
GGT: assaying, for example, by means of a “gamma-glutamyltransferase assay standardized against Szasz” (Roche Diagnostics).
Bilirubin: assaying, for example, by means of a “Bilirubin assay” (Jendrassik-Grof method) (Roche Diagnostics).
ALP: assaying, for example, by means of “ALP IFCC” (Roche Diagnostics).
ALT: assaying, for example, by “ALT IFCC” (Roche Diagnostics).
AST: assaying, for example, by means of “AST IFCC” (Roche Diagnostics). Sodium: assaying, for example, by means of “Sodium ion selective electrode” (Roche Diagnostics).
Glycemia: assaying, for example, by means of “glucose GOD-PAP” (Roche Diagnostics).
Triglycerides: assaying, for example, by means of “triglycerides GPO-PAP” (Roche Diagnostics).
Urea, GGT, bilirubin, alkaline phosphatases, sodium, glycemia, ALT and AST can be assayed on an analyzer, for example, a Hitachi 917, Roche Diagnostics GmbH, D-68298 Mannheim, Germany.
Gamma-globulins, albumin and alpha-2 globulins: assaying on protein electrophoresis, for example: capillary electrophoresis (Capillarys), SEBIA 23, rue M Robespierre, 92130 Issy Les Moulineaux, France.
ApoA1: assaying, for example, by means of “Determination of apolipoprotein A-1” (Dade Behring) with an analyzer, for example: BN2 Dade Behring Marburg GmbH, Emil von Behring Str. 76, D-35041 Marburg, Germany.
TIMP1: assaying, for example, by means of TIMP1-ELISA, Amersham.
MMP2: assaying, for example, by means of MMP2-ELISA, Amersham.
YKL-40: assaying, for example, by means of YKL-40 Biometra, YKL-40/8020, Quidel Corporation.
PIIIP: assaying, for example, by means of PIIIP RIA kit, OCFKO7-PIIIP, cis bio international.

For the biomarkers measured in the method of the present invention, the values obtained may be expressed in:
mg/dl, such as, for example, for alpha2-macroglobulin (A2M),
μg/l, such as, for example, for hyaluronic acid (HA or hyaluronate), or ferritin,
g/l, such as, for example, for apolipoprotein A1 (ApoA1), gamma-globulins (GLB) or albumin (ALB),
U/ml, such as, for example, for type III procollagen N-terminal propeptide (P3P),
IU/l, such as, for example, for gamma-glutamyltranspeptidase (GGT), aspartate aminotransferases (AST), alanine aminotransferases (ALT) or alkaline phosphatases (ALP),
μmol/l, such as, for example, for bilirubin,
Giga/l, such as, for example, for platelets (PLT),
%, such as, for example, for prothrombin time (PT),
mmol/l, such as, for example, for triglycerides, urea, sodium (NA), glycemia, or
ng/ml, such as, for example, for TIMP1, MMP2, or YKL-40.

Examples of clinical data include, but are not limited to, weight, height, body mass index, age, sex, hip perimeter, abdominal perimeter or height, spleen diameter (preferably by abdominal imaging), and mathematical combinations thereof, such as, for example, the ratio thereof, such as for example hip perimeter/abdominal perimeter.

Examples of non-invasive binary markers include, but are not limited to, diabetes, SVR (wherein SVR stands for sustained virologic response, and is defined as aviremia 6 weeks, preferably 12 weeks, more preferably 24 weeks after completion of antiviral therapy for chronic hepatitis C virus (HCV) infection), etiology, hepatic encephalopathy, ascites, and NAFLD. Regarding the binary marker “etiology”, the skilled artisan knows that said variable is a single or multiple binary marker, and that for liver disorders, etiology may be NAFLD, alcohol, virus or other. Thus, the binary marker might be expressed as NAFLD vs others (single binary marker) or as NAFLD vs reference etiology plus virus vs reference etiology and so on (multiple binary marker).

Preferably, the data is an elastometry data, preferably Liver Stiffness Evaluation (LSE) data or spleen stiffness evaluation, which may be for example obtained by VCTE or ARFI or SSI or another elastometry technique. According to a preferred embodiment of the invention, the physical data is liver stiffness measurement (LSM), preferably measured by VCTE.

In a particular embodiment, the physical data is Liver stiffness measurement (LSM) by VCTE (also known as Fibroscan™, Paris, France), preferably performed with the M probe. Preferably, examination conditions are those recommended by the manufacturer, with the objective of obtaining at least 3 and preferably 10 valid measurements. Results may be expressed as the median (kilopascals) of all valid measurements, or as IQR or as the ratio (IQR/median).

In one embodiment, step (a) of the non-invasive method of the invention comprises carrying out a VCTE (also known as Fibroscan™).

In one embodiment, step (a) of the non-invasive method of the invention comprises obtaining a liver stiffness measurement (LSM) by VCTE (also known as Fibroscan™).

In one embodiment, step (a) of the non-invasive method of the invention comprises carrying out a VCTE (also known as Fibroscan™) and optionally measuring the platelet count in a blood sample from said patient.

In one embodiment, step (a) of the non-invasive method of the invention comprises carrying out a VCTE (also known as Fibroscan™) and measuring the platelet count in a blood sample from said patient.

In one embodiment, the realization of a VCTE (also known as Fibroscan™) and the measurement of the platelet count and the comparison of the values obtained with cut-offs for assessing the presence and/or severity of varices corresponds to a PlFS algorithm.

Example 4 provides examples of PlFS algorithms.

In one embodiment, the blood test of the invention corresponds to a blood test selected from the group comprising ELF, FibroSpect™, APRI, FIB-4, Hepascore, Fibrotest™, FibroMeter™ (such as, for example, FibroMeter for cause, FibroMeter^{V2G} or FibroMeter^{V3G}), CirrhoMeter™ (such as, for example, CirrhoMeter^{V2G} or CirrhoMeter^{V3G}), CombiMeter, Elasto-FibroMeter™ (such as, for example, Elasto-FibroMeter^{V2G}), InflaMeter™, Actitest, QuantiMeter, P2/MS score, Elasto-Fibrotest, and Child-Pugh score. As these blood tests are diagnostic tests, they can be based on multivariate mathematical combination, such as, for example, binary logistic regression, or include clinical data.

ELF is a blood test based on hyaluronic acid, P3P, TIMP-1 and age.

FibroSpect™ is a blood test based on hyaluronic acid, TIMP-1 and A2M.

APRI is a blood test based on platelet and AST.

FIB-4 is a blood test based on platelet, AST, ALT and age.

HEPASCORE is a blood test based on hyaluronic acid, bilirubin, alpha2-macroglobulin, GGT, age and sex.

FIBROTEST™ is a blood test based on alpha2-macroglobulin, haptoglobin, apolipoprotein A1, total bilirubin, GGT, age and sex.

FIBROMETER™ and CIRRHOMETER™ together form a family of blood tests, the content of which depends on the cause of chronic liver disease and the diagnostic target (such as, for example, fibrosis, significant fibrosis or cirrhosis). This blood test family is called FM family and is detailed in the table below.

| | Variables | | | | | | | | | | | | |
|---|---|---|---|---|---|---|---|---|---|---|---|---|---|
| Cause | Age | Sex | Weigth | A2M | HA | PI | PLT | AST | Urea | GGT | ALT | Fer | Glu |
| Virus | | | | | | | | | | | | | |
| FM V 1G | x | | | x | x | x | x | x | x | | | | |
| FM V 2G | x | x | | x | x | x | x | x | x | | | | |
| CM V 2G | x | x | | x | x | x | x | x | x | | | | |
| FM V 3G^{a} | x | x | | x | | x | x | x | x | x | | | |
| CM V 3G^{a} | x | x | | x | | x | x | x | x | x | | | |
| Alcohol | | | | | | | | | | | | | |
| FM A 1G | x | | | x | x | x | | | | | | | |
| FM A 2G | | | | x | x | x | | | | | | | |
| NAFLD | | | | | | | | | | | | | |
| (steatosis) | | | | | | | | | | | | | |
| FM S | x | | x | | | | x | x | | | x | x | x |

FM: FibroMeter,
CM: CirrhoMeter
A2M: alpha-2 macroglobulin,
HA: hyaluronic acid,
PI: prothrombin index,
PLT: platelets,
Fer: ferritin,
Glu: glucose
^{a}HA is replaced by GGT

COMBIMETER™ or Elasto-FibroMeter™ is a family of tests based on the mathematical combination of variables of the FM family (as detailed in the Table hereinabove) or of the result of a test of the FM family with VCTE (FIBROSCAN™) result. In one embodiment, said mathematical combination is a binary logistic regression.

In one embodiment, CombiMeter™ or Elasto-FibroMeter™ results in a score based on the mathematical combination of physical data from liver or spleen elastometry such as dispersion index from VCTE (Fibroscan™) such as IQR or IQR/median or median of LSM, preferably of LSM (by Fibroscan™) median with at least 3, 4, 5, 6, 7, 8 or 9 biomarkers and/or clinical data selected from the list comprising glycemia, total cholesterol, HDL cholesterol (HDL), LDL cholesterol (LDL), AST (aspartate aminotransferase), ALT (alanine aminotransferase), AST/ALT, AST.ALT, ferritin, platelets (PLT), AST/PLT, prothrombin time (PT) or prothrombin index (PI), hyaluronic acid (HA or hyaluronate), haemoglobin, triglycerides, alpha-2 macroglobulin (A2M), gamma-glutamyl transpeptidase (GGT), urea, bilirubin, apolipoprotein A1 (ApoA1), type III procollagen N-terminal propeptide (P3NP), gamma-globulins (GBL), sodium (Na), albumin (ALB), ferritine (Fer), glucose (Glu), alkaline phosphatases (ALP), YKL-40 (human cartilage glycoprotein 39), tissue inhibitor of matrix metalloproteinase 1 (TIMP-1), TGF, cytokeratine 18 and matrix metalloproteinase 2 (MMP-2) to 9 (MMP-9), haptoglobin, diabetes, weight, body mass index, age, sex, hip perimeter, abdominal perimeter or height and ratios and mathematical combinations thereof.

INFLAMETER™ is a companion test reflecting necro-inflammatory activity including ALT, A2M, PI, and platelets.

ACTITEST is a blood test based on alpha2-macroglobulin, haptoglobin, apolipoprotein A1, total bilirubin, GGT, ALT, age and sex.

QUANTIMETER is a blood test based on (i) alpha2-macroglobulin, hyaluronic acid, prothrombin time, platelets when designed for alcoholic liver diseases, (ii) hyaluronic acid, prothrombin time, platelets, AST, ALT and glycemia when designed for NAFLD, or (iii) alpha2-macroglobulin, hyaluronic acid, platelets, urea, GGT and bilirubin when designed for chronic viral hepatitis.

P2/MS is a blood test based on platelet count, monocyte fraction and segmented neutrophil fraction.

CHILD-PUGH SCORE is a blood test based on total bilirubin, serum albumin, PT or INR, ascites and hepatic encephalopathy.

ELASTO-FIBROTEST is a test based on the mathematical combination of variables of FIBROTEST or of the result of a FIBROTEST, with LSM measurement, measured for example by Fibroscan™.

In one embodiment, step (a) of the non-invasive method of the invention comprises measuring and combining in a mathematical function the variables of ELF, i.e. hyaluronic acid, P3P, TIMP-1 and age.

In one embodiment, step (a) of the non-invasive method of the invention comprises measuring and combining in a mathematical function the variables of FibroSpect™, i.e. hyaluronic acid, TIMP-1 and A2M.

In one embodiment, step (a) of the non-invasive method of the invention comprises measuring and combining in a mathematical function the variables of APRI, i.e. platelet and AST.

In one embodiment, step (a) of the non-invasive method of the invention comprises measuring and combining in a mathematical function the variables of FIB-4, i.e. platelet, AST, ALT and age.

In one embodiment, step (a) of the non-invasive method of the invention comprises measuring and combining in a mathematical function the variables of HEPASCORE, i.e. hyaluronic acid, bilirubin, alpha2-macroglobulin, GGT, age and sex.

In one embodiment, step (a) of the non-invasive method of the invention comprises measuring and combining in a mathematical function the variables of FIBROTES™, i.e. alpha2-macroglobulin, haptoglobin, apolipoprotein A1, total bilirubin, GGT, age and sex.

In one embodiment, step (a) of the non-invasive method of the invention comprises measuring and combining in a mathematical function the variables of FIBROMETER™ and/or CIRRHOMETER™ as defined hereinabove.

In one embodiment, step (a) of the non-invasive method of the invention comprises measuring and combining in a mathematical function the variables of FIBROMETER™ and/or CIRRHOMETER™ as defined hereinabove and optionally measuring the platelet count in a blood sample from said patient.

In one embodiment, step (a) of the non-invasive method of the invention comprises measuring and combining in a mathematical function the variables of FIBROMETER™ and/or CIRRHOMETER™ as defined hereinabove and measuring the platelet count in a blood sample from said patient.

In one embodiment, step (a) of the non-invasive method of the invention comprises measuring and combining in a mathematical function the variables of CIRRHOMETER™, i.e. the following variables:
age, sex, alpha2-macroglobulin, hyaluronic acid, prothrombin time, platelets, AST and urea, or
age, sex, alpha2-macroglobulin, gamma-glutamyl transpeptidase, prothrombin time, platelets, AST and urea.

In one embodiment, step (a) of the non-invasive method of the invention comprises measuring and combining in a mathematical function the variables of CIRRHOMETER™, i.e. the following variables:
age, sex, alpha2-macroglobulin, hyaluronic acid, prothrombin time, platelets, AST and urea, or
age, sex, alpha2-macroglobulin, gamma-glutamyl transpeptidase, prothrombin time, platelets, AST and urea, and optionally measuring the platelet count in a blood sample from said patient.

In one embodiment, step (a) of the non-invasive method of the invention comprises measuring and combining in a mathematical function the variables of CIRRHOMETER™, i.e. the following variables:
age, sex, alpha2-macroglobulin, hyaluronic acid, prothrombin time, platelets, AST and urea, or
age, sex, alpha2-macroglobulin, gamma-glutamyl transpeptidase, prothrombin time, platelets, AST and urea, and measuring the platelet count in a blood sample from said patient.

In one embodiment, step (a) of the non-invasive method of the invention comprises measuring and combining in a mathematical function the variables of COMBIMETER™ or Elasto-FibroMeter™ as defined hereinabove.

In one embodiment, step (a) of the non-invasive method of the invention comprises measuring and combining in a mathematical function the variables of INFLAMETER™, i.e. ALT, A2M, PI, and platelets.

In one embodiment, step (a) of the non-invasive method of the invention comprises measuring and combining in a mathematical function the variables of ACTITEST, i.e. alpha2-macroglobulin, haptoglobin, apolipoprotein A1, total bilirubin, GGT, ALT, age and sex.

In one embodiment, step (a) of the non-invasive method of the invention comprises measuring and combining in a mathematical function the variables of QUANTIMETER, i.e. (i) alpha2-macroglobulin, hyaluronic acid, prothrombin time, platelets, (ii) hyaluronic acid, prothrombin time, platelets, AST, ALT and glycemia, or (iii) alpha2-macroglobulin, hyaluronic acid, platelets, urea, GGT and bilirubin.

In one embodiment, step (a) of the non-invasive method of the invention comprises measuring and combining in a mathematical function the variables of P2/MS score, i.e. platelet count, monocyte fraction and segmented neutrophil fraction.

In one embodiment, step (a) of the non-invasive method of the invention comprises measuring and combining in a mathematical function the variables of CHILD-PUGH SCORE, i.e. total bilirubin, serum albumin, PT or INR, ascites and hepatic encephalopathy.

In one embodiment, step (a) of the non-invasive method of the invention comprises carrying out at least two non-invasive tests for assessing the severity of a hepatic lesion or disorder, wherein said at least two non-invasive tests are different.

In one embodiment, step (a) of the non-invasive method of the invention comprises carrying out at least two non-invasive tests for assessing the severity of a hepatic lesion or disorder and optionally measuring the platelet count in a blood sample from said patient, wherein said at least two non-invasive tests are different.

In one embodiment, step (a) of the non-invasive method of the invention comprises carrying out at least two non-invasive tests for assessing the severity of a hepatic lesion or disorder and measuring the platelet count in a blood sample from said patient, wherein said at least two non-invasive tests are different.

In one embodiment, step (a) of the non-invasive method of the invention comprises carrying out at least one non-invasive test for assessing the severity of a hepatic lesion or disorder selected from the group comprising ELF, FibroSpect™, APRI, FIB-4, Hepascore, FibroMeter™, CirrhoMeter™, CombiMeter, Elasto-FibroMeter™, Elasto-Fibrotest, InflaMeter™ and VCTE (also known as Fibroscan™); and another non-invasive test for assessing the severity of a hepatic lesion or disorder selected from the group comprising ELF, FibroSpect™, APRI, FIB-4, Hepascore, FibroMeter™, CirrhoMeter™, CombiMeter, Elasto-FibroMeter™, Elasto-Fibrotest, InflaMeter™, VCTE (also known as Fibroscan™), ARFI, VTE, supersonic elastometry and MRI stiffness, wherein the at least two non-invasive tests are different.

In one embodiment, step (a) of the non-invasive method of the invention comprises carrying out at least one non-invasive test for assessing the severity of a hepatic lesion or disorder selected from the group comprising ELF, FibroSpect™, APRI, FIB-4, Hepascore, FibroMeter™, CirrhoMeter™, CombiMeter, Elasto-FibroMeter™, Elasto-Fibrotest, InflaMeter™ and VCTE (also known as Fibroscan™); and another non-invasive test for assessing the severity of a hepatic lesion or disorder selected from the group comprising ELF, FibroSpect™, APRI, FIB-4, Hepascore, FibroMeter™, CirrhoMeter™, CombiMeter, Elasto-FibroMeter™, Elasto-Fibrotest, InflaMeter™, VCTE (also known as Fibroscan™), ARFI, VTE, supersonic elastometry and MRI stiffness, and optionally measuring the platelet count in a blood sample from said patient, wherein the at least two non-invasive tests are different.

In one embodiment, step (a) of the non-invasive method of the invention comprises carrying out at least one non-invasive test for assessing the severity of a hepatic lesion or disorder selected from the group comprising ELF, FibroSpect™, APRI, FIB-4, Hepascore, FibroMeter™, CirrhoMeter™, CombiMeter, Elasto-FibroMeter™, Elasto-Fibrotest, InflaMeter™ and VCTE (also known as Fibroscan™); and another non-invasive test for assessing the severity of a hepatic lesion or disorder selected from the group comprising ELF, FibroSpect™, APRI, FIB-4, Hepascore, FibroMeter™, CirrhoMeter™, CombiMeter, Elasto-FibroMeter™, Elasto-Fibrotest, InflaMeter™, VCTE (also known as Fibroscan™), ARFI, VTE, supersonic elastometry and MRI stiffness, and measuring the platelet count in a blood sample from said patient, wherein the at least two non-invasive tests are different.

In one embodiment, step (a) of the non-invasive method of the invention comprises carrying out at least one non-invasive test for assessing the severity of a hepatic lesion or disorder selected from the group comprising ELF, FibroSpect™, APRI, FIB-4, Hepascore, FibroMeter™, CirrhoMeter™, CombiMeter, Elasto-FibroMeter™, Elasto-Fibrotest, and InflaMeter™; and another non-invasive test for assessing the severity of a hepatic lesion or disorder selected from the group comprising ELF, FibroSpect™, APRI, FIB-4, Hepascore, FibroMeter™, CirrhoMeter™, CombiMeter, Elasto-FibroMeter™, Elasto-Fibrotest, InflaMeter™, VCTE (also known as Fibroscan™), ARFI, VTE, supersonic elastometry and MRI stiffness, wherein the at least two non-invasive tests are different.

In one embodiment, step (a) of the non-invasive method of the invention comprises carrying out at least one non-invasive test for assessing the severity of a hepatic lesion or disorder selected from the group comprising ELF, FibroSpect™, APRI, FIB-4, Hepascore, FibroMeter™, CirrhoMeter™, CombiMeter, Elasto-FibroMeter™, Elasto-Fibrotest, and InflaMeter™; and another non-invasive test for assessing the severity of a hepatic lesion or disorder selected from the group comprising ELF, FibroSpect™, APRI, FIB-4, Hepascore, FibroMeter™, CirrhoMeter™, CombiMeter, Elasto-FibroMeter™, Elasto-Fibrotest, InflaMeter™, VCTE (also known as Fibroscan™), ARFI, VTE, supersonic elastometry and MRI stiffness and optionally measuring the platelet count in a blood sample from said patient, wherein the at least two non-invasive tests are different.

In one embodiment, step (a) of the non-invasive method of the invention comprises carrying out at least one non-invasive test for assessing the severity of a hepatic lesion or disorder selected from the group comprising ELF, FibroSpect™, APRI, FIB-4, Hepascore, FibroMeter™, CirrhoMeter™, CombiMeter, Elasto-FibroMeter™, Elasto-Fibrotest, and InflaMeter™; and another non-invasive test for assessing the severity of a hepatic lesion or disorder selected from the group comprising ELF, FibroSpect™, APRI, FIB-4, Hepascore, FibroMeter™, CirrhoMeter™, CombiMeter, Elasto-FibroMeter™, Elasto-Fibrotest, InflaMeter™, VCTE (also known as Fibroscan™), ARFI, VTE, supersonic elastometry and MRI stiffness and measuring the platelet count in a blood sample from said patient, wherein the at least two non-invasive tests are different.

In one embodiment, step (a) of the non-invasive method of the invention comprises carrying out at least one non-invasive test for assessing the severity of a hepatic lesion or disorder selected from the group comprising FibroMeter™, CirrhoMeter™, CombiMeter, Elasto-FibroMeter™, Elasto-Fibrotest, and InflaMeter™; and another non-invasive test for assessing the severity of a hepatic lesion or disorder selected from the group comprising, FibroMeter™, CirrhoMeter™, CombiMeter, Elasto-FibroMeter™, Elasto-Fibrotest, InflaMeter™, VCTE (also known as Fibroscan™), ARFI, VTE, supersonic elastometry and MRI stiffness, wherein said the at least two non-invasive tests are different.

In one embodiment, step (a) of the non-invasive method of the invention comprises carrying out at least one non-invasive test for assessing the severity of a hepatic lesion or disorder selected from the group comprising FibroMeter™, CirrhoMeter™, CombiMeter, Elasto-FibroMeter™, Elasto-Fibrotest, and InflaMeter™; and another non-invasive test for assessing the severity of a hepatic lesion or disorder selected from the group comprising, FibroMeter™, CirrhoMeter™, CombiMeter, Elasto-FibroMeter™, Elasto-Fibrotest, InflaMeter™, VCTE (also known as Fibroscan™), ARFI, VTE, supersonic elastometry and MRI stiffness and optionally measuring the platelet count in a blood sample from said patient, wherein the at least two non-invasive tests are different.

In one embodiment, step (a) of the non-invasive method of the invention comprises carrying out at least one non-invasive test for assessing the severity of a hepatic lesion or disorder selected from the group comprising FibroMeter™, CirrhoMeter™, CombiMeter, Elasto-FibroMeter™, Elasto-Fibrotest, and InflaMeter™; and another non-invasive test for assessing the severity of a hepatic lesion or disorder selected from the group comprising, FibroMeter™, CirrhoMeter™, CombiMeter, Elasto-FibroMeter™, Elasto-Fibrotest, InflaMeter™, VCTE (also known as Fibroscan™), ARFI, VTE, supersonic elastometry and MRI stiffness and measuring the platelet count in a blood sample from said patient, wherein the at least two non-invasive tests are different.

In one embodiment, step (a) of the non-invasive method of the invention comprises measuring and combining in a mathematical function the variables of CIRRHOMETER™, and measuring and combining in a mathematical function the variables of FIBROMETER™.

In one embodiment, step (a) of the non-invasive method of the invention comprises measuring and combining in a mathematical function the variables of CIRRHOMETER™, and measuring and combining in a mathematical function the variables of FIBROMETER™, and optionally measuring the platelet count in a blood sample from said patient.

In one embodiment, step (a) of the non-invasive method of the invention comprises measuring and combining in a mathematical function the variables of CIRRHOMETER™, and measuring and combining in a mathematical function the variables of FIBROMETER™, and measuring the platelet count in a blood sample from said patient.

In one embodiment, the method of the invention comprises carrying out a CirrhoMeter and a FibroMeter.

In one embodiment, the method of the invention comprises carrying out a CirrhoMeter and a FibroMeter and optionally measuring the platelet count in a blood sample from said patient.

In one embodiment, the method of the invention comprises carrying out a CirrhoMeter and a FibroMeter and measuring the platelet count in a blood sample from said patient.

Hence in one embodiment, the non-invasive method of the invention comprises:
(a) carrying out a CirrhoMeter and a FibroMeter, and
(b) comparing the two values obtained at step (a) with cut-offs of CirrhoMeter and FibroMeter for assessing the presence and/or severity of varices.

In one embodiment, the realization of a CirrhoMeter and a FibroMeter and the comparison of the values obtained with cut-offs of CirrhoMeter and FibroMeter for assessing the presence and/or severity of varices corresponds to a CMFM algorithm.

Examples 3 and 4 provide examples of CMFM algorithms.

In one embodiment, step (a) of the non-invasive method of the invention comprises measuring and combining in a mathematical function the variables of CIRRHOMETER™, and obtaining a liver stiffness measurement (LSM) by VCTE (also known as Fibroscan™).

In one embodiment, step (a) of the non-invasive method of the invention comprises measuring and combining in a mathematical function the variables of CIRRHOMETER™, obtaining a liver stiffness measurement (LSM) by VCTE (also known as Fibroscan™), and optionally measuring the platelet count in a blood sample from said patient.

In one embodiment, step (a) of the non-invasive method of the invention comprises measuring and combining in a mathematical function the variables of CIRRHOMETER™, obtaining a liver stiffness measurement (LSM) by VCTE (also known as Fibroscan™), and measuring the platelet count in a blood sample from said patient.

In one embodiment, the method of the invention comprises carrying out a CirrhoMeter and a VCTE (also known as Fibroscan™).

In one embodiment, the method of the invention comprises carrying out a CirrhoMeter and a VCTE (also known as Fibroscan™) and optionally measuring the platelet count in a blood sample from said patient.

In one embodiment, the method of the invention comprises carrying out a CirrhoMeter and a VCTE (also known as Fibroscan™) and measuring the platelet count in a blood sample from said patient.

Hence in one embodiment, the non-invasive method of the invention comprises:
(a) carrying out a CirrhoMeter and a VCTE (also known as Fibroscan™), and
(b) comparing the two values obtained at step (a) with cut-offs of CirrhoMeter and VCTE for assessing the presence and/or severity of varices.

In one embodiment, the realization of a CirrhoMeter and a VCTE (also known as Fibroscan™) and the comparison of the values obtained with cut-offs of CirrhoMeter and VCTE for assessing the presence and/or severity of varices corresponds to a CMFS algorithm.

Examples 2 and 4 provide examples of CMFS algorithms, including the CMFS#1 algorithm.

In one embodiment, the realization of a CirrhoMeter and a VCTE (also known as Fibroscan™) and the comparison of the values obtained with cut-offs of CirrhoMeter and VCTE for assessing the presence and/or severity of varices corresponds to the algorithm CMFS#1.

In one embodiment, the non-invasive method of the invention comprises carrying out the CMSF#1 algorithm.

In one embodiment, the realization of a CirrhoMeter and a VCTE (also known as Fibroscan™), the measurement of the platelet count and the comparison of the values obtained with cut-offs for assessing the presence and/or severity of varices corresponds to a PlCMFS algorithm.

Example 4 provides an example of PlCMFS algorithm.

In one embodiment, the method of the invention comprises carrying out a CirrhoMeter, a FibroMeter and a VCTE (also known as Fibroscan™), and measuring the platelet count.

In one embodiment, the realization of a CirrhoMeter, a FibroMeter and a VCTE (also known as Fibroscan™), the measurement of the platelet count and the comparison of the values obtained with cut-offs for assessing the presence and/or severity of varices corresponds to a PlFMCMFS algorithm.

Example 4 provides an example of PlFMCMFS algorithm. FIGS. 16 to 19 illustrate the construction of a PlFMCMFS algorithm with multiple predictive zones.

In one embodiment, step (c) of the non-invasive method of the invention comprises measuring the variables of ELF, i.e. hyaluronic acid, P3P, TIMP-1 and age.

In one embodiment, step (c) of the non-invasive method of the invention comprises measuring the variables of FibroSpect™, i.e. hyaluronic acid, TIMP-1 and A2M.

In one embodiment, step (c) of the non-invasive method of the invention comprises measuring the variables of APRI, i.e. platelet and AST.

In one embodiment, step (c) of the non-invasive method of the invention comprises measuring the variables of FIB-4, i.e. platelet, AST, ALT and age.

In one embodiment, step (c) of the non-invasive method of the invention comprises measuring the variables of HEPASCORE, i.e. hyaluronic acid, bilirubin, alpha2-macroglobulin, GGT, age and sex.

In one embodiment, step (c) of the non-invasive method of the invention comprises measuring the variables of FIBROTEST™, i.e. alpha2-macroglobulin, haptoglobin, apolipoprotein A1, total bilirubin, GGT, age and sex.

In one embodiment, step (c) of the non-invasive method of the invention comprises measuring the variables of FIBROMETER™ and/or CIRRHOMETER™ as defined hereinabove.

In one embodiment, step (c) of the non-invasive method of the invention comprises measuring the variables of CIRRHOMETER™, i.e. the following variables:
age, sex, alpha2-macroglobulin, hyaluronic acid, prothrombin time, platelets, AST and urea, or
age, sex, alpha2-macroglobulin, gamma-glutamyl transpeptidase, prothrombin time, platelets, AST and urea.

In one embodiment, step (c) of the non-invasive method of the invention comprises measuring the variables of COMBIMETER™ or Elasto-FibroMeter™ as defined hereinabove.

In one embodiment, step (c) of the non-invasive method of the invention comprises measuring the variables of INFLAMETER™, i.e. ALT, A2M, PI, and platelets.

In one embodiment, step (c) of the non-invasive method of the invention comprises measuring the variables of ACTITEST, i.e. alpha2-macroglobulin, haptoglobin, apolipoprotein A1, total bilirubin, GGT, ALT, age and sex.

In one embodiment, step (c) of the non-invasive method of the invention comprises measuring the variables of QUANTIMETER, i.e. (i) alpha2-macroglobulin, hyaluronic acid, prothrombin time, platelets, (ii) hyaluronic acid, prothrombin time, platelets, AST, ALT and glycemia, or (iii) alpha2-macroglobulin, hyaluronic acid, platelets, urea, GGT and bilirubin.

In one embodiment, step (c) of the non-invasive method of the invention comprises measuring the variables of P2/MS score, i.e. platelet count, monocyte fraction and segmented neutrophil fraction.

In one embodiment, step (c) of the non-invasive method of the invention comprises measuring the variables of CHILD-PUGH SCORE, i.e. total bilirubin, serum albumin, PT or INR, ascites and hepatic encephalopathy.

Examples of physical methods include, but are not limited to, medical imaging data and clinical measurements, such as, for example, measurement of spleen, especially spleen length (that may also be referred as diameter). According to an embodiment, the physical method is selected from the group comprising ultrasonography, especially Doppler-ultrasonography and elastometry ultrasonography and velocimetry ultrasonography (preferred tests using said data are vibration controlled transient elastography (VCTE, also known as Fibroscan™), ARFI, VTE, supersonic elastometry (supersonic imaging), MRI (Magnetic Resonance Imaging), and MNR (Magnetic Nuclear Resonance) as used in spectroscopy, especially MNR elastometry or velocimetry.

In one embodiment, the physical method is VCTE, ARFI, VTE, supersonic elastometry or MRI stiffness.

In one embodiment of the invention, the method of the invention comprises carrying out a VCTE, which refers to obtaining at least 3 and preferably 10 valid measurements and recovering a physical data corresponding to the median in kilopascals of all valid measurements.

In one embodiment, the present invention non-invasive method for assessing the presence and/or severity of esophageal varices in a hepatic disease patient comprises:
(a) carrying out a CirrhoMeter (such as, for example, a CirrhoMeter^{V2G} or a CirrhoMeter^{VV3G}, preferably a CirrhoMeter^{V2G}), resulting in a CirrhoMeter score, and
(b) comparing the CirrhoMeter score obtained at step (a) with cut-offs of said CirrhoMeter for assessing the presence and/or severity of esophageal varices, thereby determining if the patient does not present esophageal varices (preferably large esophageal varices), presents esophageal varices or is in an indeterminate zone, and
for patients in the indeterminate zone, the method of the invention further comprises:
(c) measuring the variables of CirrhoMeter in the subject,
(d) obtaining imaging data on varices status, wherein said imaging data are obtained by a non-invasive imaging method,
(e) mathematically combining
   the variables obtained in step (c), or any mathematical combination thereof in a CirrhoMeter (such as, for example, a CirrhoMeter^{V2G} or a CirrhoMeter^{V3G}, preferably a CirrhoMeter^{V2G}) with
   the data obtained at step (d),
wherein the mathematical combination results in a diagnostic score, and
(f) assessing the presence and/or severity of esophageal varices based on the diagnostic score obtained in step (e).

In one embodiment, the present invention non-invasive method for assessing the presence and/or severity of varices, selected from gastric and esophageal varices in a hepatic disease patient comprises:
(a) obtaining and mathematically combining in a CirrhoMeter (such as, for example, a CirrhoMeter^{V2G} or a CirrhoMeter^{V3G}, preferably a CirrhoMeter^{V2G}), the following variables:
   age, sex, alpha2-macroglobulin, hyaluronic acid, prothrombin time, platelets, AST and urea, or
   age, sex, alpha2-macroglobulin, gamma-glutamyl transpeptidase, prothrombin time, platelets, AST and urea
thereby obtaining a CirrhoMeter score, and
(b) comparing the CirrhoMeter score obtained at step (a) with cut-offs of said CirrhoMeter for assessing the presence and/or severity of esophageal varices, thereby determining if the patient does not present esophageal varices, presents esophageal varices (preferably large esophageal varices) or is in an indeterminate zone, and
for patients in the indeterminate zone, the method of the invention further comprises:
(c) measuring the following variables in the subject:
   age, sex, alpha2-macroglobulin, hyaluronic acid, prothrombin time, platelets, AST and urea, or
   age, sex, alpha2-macroglobulin, gamma-glutamyl transpeptidase, prothrombin time, platelets, AST and urea,
(d) obtaining imaging data on varices status, wherein said imaging data are obtained by a non-invasive imaging method,
(e) mathematically combining
   the variables obtained in step (c), or any mathematical combination thereof in a CirrhoMeter (such as, for example, a CirrhoMeter^{V2G} or a CirrhoMeter^{V3G}, preferably a CirrhoMeter^{V2G}) with
   the data obtained at step (d),
wherein the mathematical combination results in a diagnostic score, and
(f) assessing the presence and/or severity of varices selected from gastric and esophageal varices, based on the diagnostic score obtained in step (e).

Examples of non-invasive imaging data allowing the assessment of varices status (i.e. for visualizing varices or the absence of varices) include data obtained with non-invasive imaging methods or radiology.

Examples of non-invasive imaging methods for assessing varices status include, but are not limited to, esophageal capsule endoscopy (ECE), CT-scan, echo-endoscopy or MRI.

Examples of esophageal capsules that may be used in the method of the present invention includes esophageal capsules developed by Given-covidien-medtronic.

Examples of radiologic methods for assessing varices status include, but are not limited to, CT-scanner and MRI.

In one embodiment, the non-invasive imaging data corresponds to a grade according to the size of the visualized varices:
grade 0: absence of varices,
grade 1: presence of small varices of less than 5 mm in diameter or 15 to 25% of esophageal circumference, and
grade 2: presence of large varices (i.e. of at least about 5 mm in diameter or 15 to 25% of esophageal circumference).

In one embodiment, the step (a) of the method of the invention comprises carrying out a CirrhoMeter, such as, for example, a CirrhoMeter^{V2G} or a CirrhoMeter^{V3G}, preferably a CirrhoMeter^{V2G}.

In one embodiment, the step (c) of the method of the invention comprises carrying out a CirrhoMeter, such as, for example, a CirrhoMeter^{V2G} or a CirrhoMeter^{V3G}, preferably a CirrhoMeter^{V2G}.

In one embodiment, the step (a) and step (c) of the method of the invention both comprise carrying out a CirrhoMeter, such as, for example, a CirrhoMeter^{V2G} or a CirrhoMete^{V3G}, preferably a CirrhoMeter^{V2G}.

In one embodiment, the step (a) of the method of the invention comprises carrying out a FibroMeter, such as, for example, a FibroMeter^{V2G} or a FibroMeter^{V3G}.

In one embodiment, the step (c) of the method of the invention comprises carrying out a FibroMeter, such as, for example, a FibroMeter^{V2G} or a FibroMeter^{V3G}.

In one embodiment, the step (a) and step (c) of the method of the invention both comprise carrying out a FibroMeter, such as, for example, a FibroMeter^{V2G} or a FibroMeter^{V3G}.

In one embodiment, the step (d) of the method of the invention comprises obtaining imaging data obtained by ECE.

In one embodiment, the step (e) of the method of the invention comprises mathematically combining a CirrhoMeter (such as, for example, a CirrhoMeter^{V2G} or a CirrhoMeter^{V3G}, preferably a CirrhoMeter^{V2G}) or the variables of a CirrhoMeter (such as, for example, a CirrhoMeter^{V2G} or a CirrhoMeter^{VV3G}, preferably a CirrhoMeter^{V2G}) with a data obtained by ECE.

In one embodiment, the step (e) of the method of the invention comprises mathematically combining a FibroMeter (such as, for example, a FibroMeter^{V2G} or a FibroMeter^{V3G}) or the variables of a FibroMeter (such as, for example, a FibroMeter^{V2G} or a FibroMeter^{V3G}) with a data obtained by ECE.

In one embodiment, the step (c) of the method of the invention comprises carrying out a CirrhoMeter, such as, for example, a CirrhoMeter^{V2G} or a CirrhoMeter^{V3G}, preferably a CirrhoMeter^{V2G}; the step (d) of the method of the invention comprises obtaining imaging data obtained by ECE; and the step (e) of the method of the invention comprises mathematically combining the result of the CirrhoMeter carried out at step (c) with the data obtained by ECE.

In one embodiment, the step (c) of the method of the invention comprises carrying out a FibroMeter, such as, for example, a FibroMeter^{V2G} or a FibroMeter^{V3G}; the step (d) of the method of the invention comprises obtaining imaging data obtained by ECE; and the step (e) of the method of the invention comprises mathematically combining the result of the FibroMeter carried out at step (c) with the data obtained by ECE.

In one embodiment, the step (a) of the method of the invention comprises carrying out a CirrhoMeter, such as, for example, a CirrhoMeter^{V2G} or a CirrhoMeter^{V3G}, preferably a CirrhoMeter^{V2G}; the step (c) of the method of the invention comprises carrying out a CirrhoMeter, such as, for example, a CirrhoMeter^{V2G} or a CirrhoMeter^{V3G}, preferably a CirrhoMeter^{V2G}; the step (d) of the method of the invention comprises obtaining imaging data obtained by ECE; and the step (e) of the method of the invention comprises mathematically combining the result of the CirrhoMeter carried out at step (c) with the data obtained by ECE.

In one embodiment, the step (a) of the method of the invention comprises carrying out a FibroMeter, such as, for example, a FibroMeter^{V2G} or a FibroMeter^{V3G}; the step (c) of the method of the invention comprises carrying out a FibroMeter, such as, for example, a FibroMeter^{V2G} or a FibroMeter^{V3G}; the step (d) of the method of the invention comprises obtaining imaging data obtained by ECE; and the step (e) of the method of the invention comprises mathematically combining the result of the FibroMeter carried out at step (c) with the data obtained by ECE.

In one embodiment, the step (a) of the method of the invention comprises carrying out a FibroMeter, such as, for example, a FibroMeter^{V2G} or a FibroMeter^{V3G}; the step (c) of the method of the invention comprises carrying out a CirrhoMeter, such as, for example, a CirrhoMeter^{V2G} or a CirrhoMeter^{V3G}, preferably a CirrhoMeter^{V2G}; the step (d) of the method of the invention comprises obtaining imaging data obtained by ECE; and the step (e) of the method of the invention comprises mathematically combining the result of the CirrhoMeter carried out at step (c) with the data obtained by ECE.

In one embodiment, the step (a) of the method of the invention comprises carrying out a CirrhoMeter, such as, for example, a CirrhoMeter^{V2G} or a CirrhoMeter^{V3G}, preferably a CirrhoMeter^{V2G}; the step (c) of the method of the invention comprises carrying out a FibroMeter, such as, for example, a FibroMeter^{V2G} or a FibroMeter^{V3G}; the step (d) of the method of the invention comprises obtaining imaging data obtained by ECE; and the step (e) of the method of the invention comprises mathematically combining the result of the FibroMeter carried out at step (c) with the data obtained by ECE.

In one embodiment, the patient is a mammal, preferably a human. In one embodiment, the patient is a male or a female. In one embodiment, the patient is an adult or a child.

In one embodiment, the patient is affected, preferably is diagnosed with a liver disease or disorder.

In one embodiment, the patient is affected with a liver disease or disorder, preferably selected from the list comprising significant porto-septal fibrosis, severe porto-septal fibrosis, centrolobular fibrosis, cirrhosis, persinusoidal fibrosis, the fibrosis being from alcoholic or non-alcoholic origin.

In one embodiment, the patient is affected with a chronic disease, preferably said chronic disease is selected from the group comprising chronic viral hepatitis C, chronic viral hepatitis B, chronic viral hepatitis D, chronic viral hepatitis E, non-alcoholic fatty liver disease (NAFLD), alcoholic chronic liver disease, autoimmune hepatitis, primary biliary cirrhosis, hemochromatosis and Wilson disease.

In one embodiment, the subject is a cirrhotic patient. In one embodiment, the patient was previously diagnosed as cirrhotic by any method known in the art, including invasive (e.g. biopsy) or non-invasive (e.g. blood test or physical method) methods already disclosed in the art.

In one embodiment of the invention, the mathematical combination is a combination within a mathematical function selected from a binary logistic regression, a multiple linear regression or any multivariate analysis. One skilled in the art may found in the prior art all information related to the mathematical function.

In one embodiment, the mathematical function is a logistic regression. A logistic regression produces a formula in the form:

score=a₀+a₁x₁+a₂x₂+ . . .

wherein the coefficients aᵢ are constants and the variables xᵢ are the variables (preferably independent variables).

Preferably, the mathematical function is a binary logistic regression where final score is 1/1-e^{score}.

In one embodiment, the diagnostic method of the invention presents:
a NPV (or sensitivity) of at least about 75%, preferably of at least about 80%, more preferably of at least about 85%, 86%, 87%, 88%, 89%, 90%, 91%, 92%, 93%, 94%, 95%, 96%, 97%, 98%, 99% or more, and/or
a PPV (or specificity) of at least about 75%, preferably of at least about 80%, preferably of at least about 85%, 86%, 87%, 88%, 89%, 90%, 91%, 92%, 93%, 94%, 95%, 96%, 97%, 98%, 99% or more.

In one embodiment, the diagnostic method of the invention presents a NPV of at least 95% and/or a PPV of at least 90%.

In one embodiment, the diagnostic method of the invention presents a diagnostic performance (patients correctly classified or AUROC) for esophageal varices, preferably for large esophageal varices, of at least about 0.89, preferably of at least about 0.90, 0.91, 0.92, 0.93, 0.94, 0.95, 0.96, 0.97, 0.98, 0.99 or more.

In one embodiment, the percentage of correctly classified patients using the method of the invention is of at least about 90%, preferably of at least about 90.5, 91, 91.5, 92, 92.5, 93, 93.5, 94, 94.5, 95, 95.5, 96, 96.5, 97, 97.5, 98, 98.5, 99, 99.5 or more.

In one embodiment, the diagnostic method of the invention presents a specificity of at least 90%, preferably of at least 91, 92, 93, 94, 95, 96, 97, 98, 99% or more. In one embodiment, the diagnostic method of the invention presents a specificity of 100%.

In one embodiment, using the diagnostic method of the invention, an invasive test for determining the presence or absence of esophageal varices, such as, for example, endoscopy (UGIE) is required in at most about 50%, preferably in at most about 45, 40, 35, 30, 25, 20, 15, 10% or less of the hepatic disease patients.

In one embodiment, using the diagnostic method of the invention, the rate of saved UGIE is of at least about 20%, preferably of at least about 30, 40, 50, 60, 70, 80, 90% or more.

In one embodiment, using the diagnostic method of the invention, the rate of missed large esophageal varices is of at most about 20%, preferably of at most about 19, 18, 17, 16, 15, 14, 13, 12, 11, 10, 9, 8, 7, 6, 5, 4, 3, 2, 1% or less.

Another object of the invention is a non-invasive method for assessing the presence and/or severity of varices, selected from gastric and esophageal varices in a liver disease patient, preferably in a patient with chronic liver disease, wherein said method comprises:
i. measuring at least one of the following variables from the subject:
   biomarkers,
   clinical data,
   binary markers,
   physical data from medical imaging or clinical measurement,
ii. obtaining imaging data on varices status, wherein said imaging data are obtained by a non-invasive imaging method,
iii. mathematically combining, preferably in a binary logistic regression,
   the variables obtained in step (i), or any mathematical combination thereof with,
   the data obtained at step (ii),
   wherein the mathematical combination results in a diagnostic score, and
iv. assessing the presence and/or severity of varices, selected from gastric and esophageal varices based on the diagnostic score obtained in step (iii).

In one embodiment, the biomarkers, clinical data, binary markers, physical data and imaging data on varices status are as defined hereinabove.

In one embodiment, the variables measured in step (i) are the variables of a CirrhoMeter (such as, for example, a CirrhoMeter^{V2G} or a CirrhoMeter^{V3G}, preferably a CirrhoMeter^{V2G}).

In another embodiment, the variables measured in step (i) are the variables of a FibroMeter (such as, for example, a FibroMeter^{V2G} or a FibroMeter^{V3G}).

In one embodiment, the imaging data are obtained in step (ii) by ECE.

In one embodiment, the variables obtained in step (i) are mathematically combined in a non-invasive diagnostic test, preferably in a score, prior to the mathematical combination with the data obtained at step (ii). In one embodiment, the variables obtained in step (i) are mathematically combined in a FibroMeter or in a CirrhoMeter.

In one embodiment, the step (iii) of the method of the invention comprises mathematically combining a CirrhoMeter (such as, for example, a CirrhoMeter^{V2G} or a CirrhoMeter^{V3G}, preferably a CirrhoMeter^{V2G}) or the variables of a CirrhoMeter (such as, for example, a CirrhoMeter^{V2G} or a CirrhoMeter^{V3G}, preferably a CirrhoMeter^{V2G}) with a data obtained by ECE.

In one embodiment, the step (iii) of the method of the invention comprises mathematically combining a FibroMeter (such as, for example, a FibroMeter^{V2G} or a FibroMeter^{V3G}) or the variables of a FibroMeter (such as, for example, a FibroMeter^{V2G} or a FibroMeter^{V3G}) with a data obtained by ECE.

In one embodiment, the patient was previously diagnosed with a cirrhosis.

In one embodiment, the patient was classified in the indeterminate zone according to the step (b) of the method as defined hereinabove.

In one embodiment of the invention, the method of the invention is computer implemented.

The present invention thus also relates to a microprocessor comprising a computer algorithm carrying out the prognostic method of the invention.

The skilled artisan would easily deduce that the method of the invention being indicative of the presence of varices, selected from gastric and esophageal varices, especially of large esophageal varices, it may be used by the physician willing to provide the best medical care to his/her patient. For example, a patient presenting varices, selected from gastric and esophageal varices will require treatment of said varices, while a patient without esophageal varices will be subjected to yearly surveillance of varices. On the other hand, a patient diagnosed in the indeterminate zone regarding the value of the diagnostic score of the invention may require an UGIE endoscopy in order to assess the presence or absence of varices.

Therefore, the present invention also relates to a method for adapting the treatment, the medical care or the follow-up of a patient, wherein said method comprises implementing the non-invasive method of the invention.

The present invention also relates to a method for monitoring the treatment of a patient, wherein said method comprises implementing the non-invasive method of the invention, thereby assessing the appearance of esophageal varices in a patient.

The present invention also relates to a method for treating a hepatic disease patient, wherein said method comprises (i) implementing the non-invasive method of the invention and (ii) treating the patient according to the value obtained by the patient.

### BRIEF DESCRIPTION OF THE DRAWINGS

FIG. 1 is a graphic representation of the study design in Example 1. Roles (oblique grey characters) of populations (horizontal bars), main investigations performed (vertical bars) and objectives (horizontal black characters). ECE: esophageal capsule endoscopy, LEV: large esophageal varices, NPV: negative predictive value, PPV: positive predictive value, UGI: upper gastro-intestinal, VCTE: vibration control transient elastography.

FIG. 2 is a graphic representation of the different strategies evaluated for LEV diagnosis in Example 1. Among combinations, there were several possibilities but only the most clinically relevant were selected for evaluation (see table 6). ECE: esophageal capsule endoscopy, VCTE: vibration control transient elastography (Fibroscan).

FIG. 3 is a combination of graphs showing diagnostic indices of non-invasive tests for large esophageal varices in derivation population. Two opposite examples: panel A shows the best score with large (in terms of patient proportion) zones of negative (NPV in light blue)) and positive predictive (PPV in dark green) values at 100%. Panel B shows VCTE (Fibroscan) with a low maximum PPV (<40%), i.e. no clinically interesting PPV zone. Panels C and D show the scores of the best clinically applicable strategy; note that the combination markedly improved the NPV≥95% zone and the PPV≥90% zone compared to CirrhoMeter^{VIRUS2G} score. Se: sensitivity, Spe: specificity, DA: diagnostic accuracy, ECE: esophageal capsule endoscopy. Vertical figures on X axis indicate ranked patient values.

FIG. 4 is a histogram showing the relationship between CirrhoMeter^{VIRUS2G} fibrosis classes (X axis), Metavir fibrosis (F) stages and large esophageal varices (Y axis) in validation population #1 with chronic liver disease (Example 1). Note that LEVs were only present in Metavir F4 stage and that LEVs were more frequent in Metavir F4 classified as F4 than F3/4 by CirrhoMeter^{VIRUSS2G}.

FIG. 5 is a scatter plot of CirrhoMeter^{VIRUS2G} score (X axis) with CirrhoMeter^{VIRUS2G}+ECE score (Y axis) as a function of LEV by endoscopy (UGIE) in the derivation population (Example 1). The three curves are determined by ECE: no EV in bottom curve, small EV in intermediate curve and large EV in top curve. This figure clearly indicates that two patients with LEV without EV on ECE are rescued by the combination of CirrhoMeter^{VIRUS2G} to ECE (lower right corner of zone 2B). Each score is divided into 3 zones according to high predictive value cut-offs. In VariScreen algorithm, CirrhoMeter^{VIRUS2G} is performed first. ECE is then performed in indeterminate CirrhoMeter^{VIRUS2G} zone 2. Thus, VariScreen zones are: LEV absence=zones 1+2A, LEV presence=zones 3+2 C. Then, UGIE is performed in indeterminate VariScreen zone 2B. Figures x/y denote number of patients with LEV among all patients in each of the 9 zones determined by combination of the two tests.

FIG. 6 shows the VariScreen algorithm for large esophageal varices according to the study presented in Example 1. CirrhoMeter^{VIRUS2G} is performed in all patients. Those below the CirrhoMeter^{VIRUS2G} NPV cut-off for large EV have a 98-99% NPV for LEV. Those beyond the CirrhoMeter^{VIRUS2G} PPV cut-off for large EV have a 83% PPV for LEV. Those patients between the two CirrhoMeter^{VIRUS2G} cut-offs are offered ECE. Then, the ECE+CirrhoMeter^{VIRUS2G} score is calculated in previous selected patients. Those below the NPV cut-off for LEV of ECE+CirrhoMeter^{VIRUS2G} score have a 98-99% NPV for LEV. Those beyond the score PPV cut-off have a 90% PPV for LEV (detail not shown). Those patients between the two score cut-offs are offered endoscopy. ECE: esophageal capsule endoscopy, EV: esophageal varices.

FIG. 7 is a histogram comparing all 4 strategies based on esophageal capsule endoscopy and/or CirrhoMeter^{VIRUS2G} in derivation population. Figures inside bars indicate measured predictive values; figures above arrows indicate p value. Arrows indicate significant pairwise differences. Missed LEV are expressed here in proportion of all patients. LEV: large esophageal varices, UGIE: upper gastro-intestinal endoscopy, NS: not significant.

FIG. 8 is a scheme illustrating the hypothesis for large esophageal varices (LEV) screening tested in Example 3. In the classical attitude, upper gastrointestinal endoscopy (UGIE) is performed in every cirrhotic patient. However, UGIE is probably overused for LEV screening since the threshold for LEV is subsequent to the cirrhosis cut-off. In the current attitude, where the target of the non-invasive test is cirrhosis, there is a grey zone for cirrhosis diagnosis that aggravates UGIE overuse. This suggests that the best strategy for LEV screening is to apply the LEV cut-off of the non-invasive test to all patients with chronic liver disease irrespective of cirrhosis diagnosis.

FIG. 9 is a combination of a scatter plot and a scheme. (A) Scatter plot of CirrhoMeter score (X axis) with (CirrhoMeter+ECE) score (Y axis) as a function of esophageal varice (EV) grade (symbols: +▴▪) by endoscopy (UGIE) determining the VariScreen algorithm in the derivation population. The three oblique curves were due to the EV grades by esophageal capsule endoscopy (ECE) included in the (CirrhoMeter+ECE) score. Both axes are divided into three predictive zones (rectangles determined by vertical lines for CirrhoMeter and horizontal lines for (CirrhoMeter+ECE) score) according to high predictive value cut-offs. Practically, CirrhoMeter is performed first. Thereafter, ECE is performed in the indeterminate CirrhoMeter zone (light grey area). Finally, UGIE is performed in the indeterminate (CirrhoMeter+ECE) zone (dark grey area). The plot shows the advantages of VariScreen over ECE: three patients falsely negative on ECE had in fact large EV (LEV) on UGIE (arrows, 13 other false negatives are not arrowed) and two out of five patients falsely positive for LEV on ECE (arrows) were rescued by UGIE. The VariScreen algorithm missed two patients with LEV (arrows). Note that the VariScreen algorithm presented here (and described in Example 3) is another version of the VariScreen algorithm presented in FIG. 5 (and described in Example 1). (B) Scheme summarizing the three VariScreen zones derived from the scatter plot of (A): LEV ruled out zone (left and bottom), LEV ruled in zone (top), and indeterminate zone (middle, light grey) where UGIE is indicated. The tests determining the zones are shown in grey characters. The dashed rectangle illustrates an indication for ECE within the indeterminate CirrhoMeter zone.

FIG. 10 is a scheme illustrating the VariScreen algorithm for large esophageal varices (LEV) as described in Example 3. Note that the VariScreen algorithm of Example 3 is another version of the VariScreen algorithm of Example 1 presented in FIG. 6. ECE: esophageal capsule endoscopy.

FIG. 11 is a combination of graphs illustrating the FibroMeter+CirrhoMeter algorithm for large esophageal varices (LEV) as performed in Example 3. (A) FibroMeter+CirrhoMeter algorithm performed on the derivation population. (B) FibroMeter+CirrhoMeter algorithm performed on the validation population. LEV ruled out (NPV) zone (as shown), LEV ruled in (PPV) zone (as shown), and indeterminate zone (grey) where UGIE is indicated.

FIG. 12 is a graph showing the curves of negative predictive value (NPV) and positive predictive value (PPV) (Y axis) for large esophageal varices in cirrhosis as a function of Fibroscan values (X axis). Note that in this case, there is a large 95% NPV zone but no useful PPV zone since the maximum PPV is <40%.

FIG. 13 is a scheme depicting the NPV, PPV and indeterminate zones obtained with a single diagnostic test. Note that the PPV zone is usually smaller than the NPV zone.

FIG. 14 is a scatter plot showing the NPV, PPV and indeterminate zones obtained with two diagnostic tests. Note that in this case, the cut-offs for NPV and PPV zones were chosen for a NPV and PPV of 100%. For example, the cut-off of CirrhoMeter (Y axis) was at around 0.35 and that of Fibroscan (X axis) at around 35 for 100% NPV.

FIG. 15 is a scatter plot illustrating the construction of predictive zones obtained with two diagnostic tests. Different NPV zones obtained with the NPV cut-offs of said two diagnostic tests and/or combinations of the NPV cut-offs of said two diagnostics are shown (see NPV zones 1 to 5).

FIG. 16 is a scatter plot illustrating the first step of the PlFMCMFS#1 algorithm with NPV and PPV zones obtained using two diagnostic tests: platelets (Y axis) and Fibroscan (X axis) for the diagnosis of large esophageal varices in the original reference population of patients with cirrhosis.

FIG. 17 is a scatter plot illustrating the second step of the PlFMCMFS#1 algorithm with NPV and PPV zones obtained using two diagnostic tests: CirrhoMeter (Y axis) and Fibroscan (X axis) for the diagnosis of large esophageal varices in the sub-population of cirrhosis where patients located in the NPV zone of FIG. 16 (step 1) were excluded. This is the first additional predictive zone.

FIG. 18 is a scatter plot illustrating the final (initial and additional) NPV and PPV zones obtained with several diagnostic tests included in the PlFMCMFS#1 algorithm with a projection on the scatterplot of CirrhoMeter×Fibroscan (first additional zone: see FIG. 17) as a function of algorithm zones. The scatterplot of platelets x Fibroscan was used for the first (initial) NPV zone (see FIG. 16). Other additional zones with other test combinations are included in the algorithm but test contribution cannot be easily shown in a two dimensional graph. The zones rescued correspond to the improvements brought by additional predictive zones. The mixed NPV zone corresponds to a zone where additional NPV zones are partially included.

FIG. 19 is a scatter plot illustrating the final (initial and additional) NPV and PPV zones obtained with several diagnostic tests included in the PlFMCMFS#1 algorithm with a projection on the scatterplot of CirrhoMeter x Fibroscan (first additional zone: see FIG. 16) as a function of large esophageal varices. This figure is aimed to be compared with FIG. 18 in order to check the algorithm accuracy. CM: CirrhoMeter, VCTE: Fibroscan.

### EXAMPLES

The present invention is further illustrated by the following examples.

### Example 1

### Patients and Methods

### Patient Populations

Diagnostic strategy development needed a derivation population where all diagnostic tests were available, especially esophageal capsule endoscopy (ECE). For that purpose, we disposed of a population with cirrhotic patients. The derivation population was extracted from a prospective study comparing ECE and upper gastro-intestinal endoscopy (UGIE) in the esophageal varices (EV) diagnosis in patients with cirrhosis with various causes (Sacher-Huvelin S, et al, Endoscopy 2015: (in press: PMID: 25730284)). We included the 287 patients having both ECE and UGIE.

Validation populations were already published for the evaluation of non-invasive fibrosis tests (except population #4). The two main differences with derivation population were (i) the availability of liver biopsy in all patients and (ii) that all fibrosis stages were represented.

Validation of diagnostic strategy required a chronic liver disease (CLD) population with UGIE for reference and blood tests. Briefly, this validation population #1 was particular for several reasons. Patients with CLD attributed to virus or alcohol could have decompensated cirrhosis and had UGIE even in non-cirrhotic patients (Oberti F et al, Gastrointest Endosc 1998; 48:148-157). Finally, we considered 3 additional large validation CLD populations #2 to #4 to mainly validate specificity robustness. Validation populations #2 (Pascal J P et al, N Engl J Med 1987; 317:856-861) and #3 (Castera L et al, J Hepatol 2008; 48:835-847) comprised patients with CLD due to chronic hepatitis C (CHC) without liver complication.

Population #4 comprised patients with CLD due to non-alcoholic fatty liver disease (NAFLD) without liver complication. Patients with biopsy-proven NAFLD were consecutively included in the study from January 2004 to June 2014 at Angers University Hospital and from October 2003 to April 2014 at Bordeaux University Hospital. NAFLD was defined as liver steatosis on liver biopsy after exclusion of concomitant steatosis-inducing drugs, excessive alcohol consumption (>210 g/week in men or >140 g/week in women), chronic hepatitis B or C infection, and histological evidence of other concomitant chronic liver disease. Patients were excluded if they had cirrhosis complications (ascites, variceal bleeding, systemic infection, or hepatocellular carcinoma). The study protocol conformed to the ethical guidelines of the current Declaration of Helsinki and all patients gave informed written consent.

### Study Design

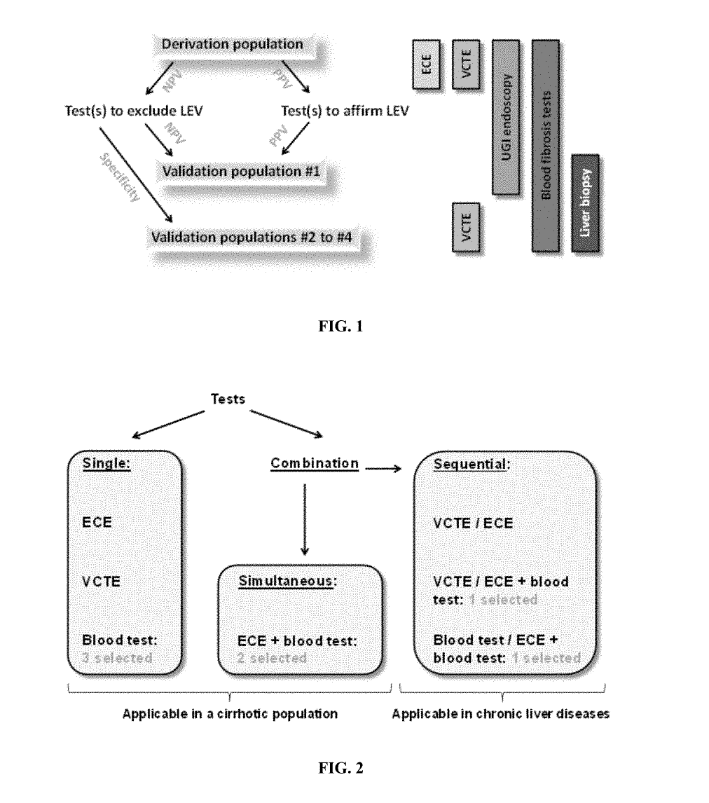

Study design is summarized in table 1 and FIG. 1.

TABLE 1
Main characteristics of populations.

| | | Validation | | | |
|---|---|---|---|---|---|
| | Derivation | #1 | #2 | #3 | #4 |
| Cause | Miscellaneous | Alcohol, | Virus C | Virus C | NAFLD |
| | | virus | | | |
| Fibrosis | Cirrhosis | All stages | All stages | All | All |
| spectrum | | | | stages | stages |
| Endoscopy | Yes | Yes | No | No | No |
| ECE | Yes | No | No | No | No |
| Blood tests | Yes | Yes | Yes | Yes | Yes |
| VCTE | Yes | No | No | Yes | Yes |
| Liver biopsy | No | Yes | Yes | Yes | Yes |

### Diagnostic Algorithms

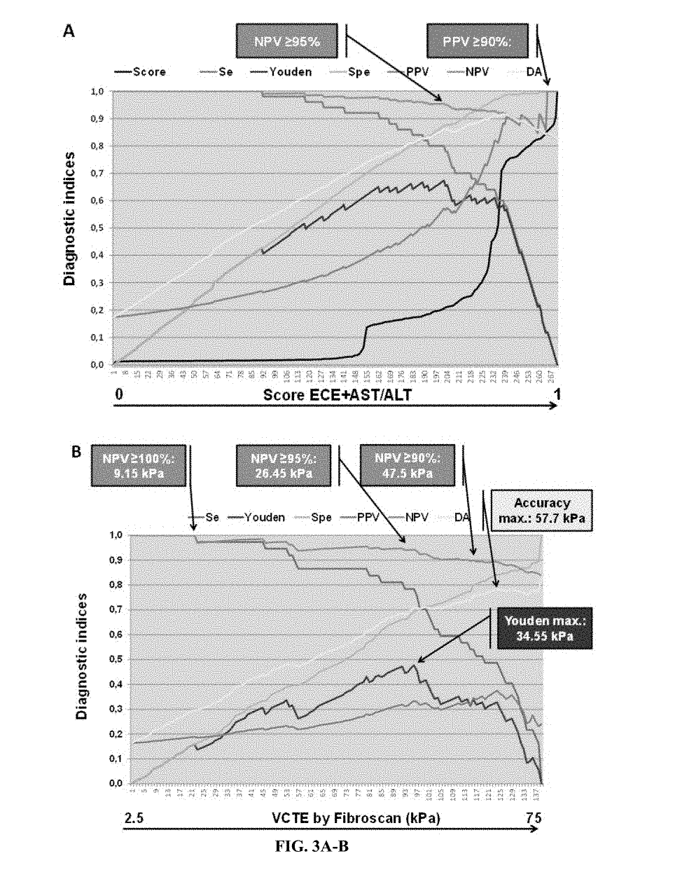

The diagnostic algorithms included different strategies (FIG. 2).

The first ones comprised a single diagnostic test. The second ones combined several tests. These combinations were either symmetric, i.e. the same test combination for both predictive values (PV), or asymmetric to reach higher PV, i.e. with different tests for negative predictive value (NPV) and positive predictive value (PPV). We defined a clinically applicable strategy as including necessarily a low constraint test (e.g. blood test) to exclude LEV (usually in asymptomatic patient) and possibly a high constraint test (e.g. UGIE) to affirm LEV (in the most severe patients).

### Strategy Selection

First step—We evaluated available strategies combining one or several tests to determine the most accurate strategy in the derivation population irrespective of clinical applicability in order to determine the most accurate strategy as paradigm.

Second step—We concentrated on the sole clinically applicable asymmetric strategies. When we compared strategies, we had no single comparator and a choice had to be based on a balance between the best three indicators (patient proportion with PV, saved UGIE and missed LEV, see below) according to statistical comparisons.

### Diagnostic Tools

### Endoscopic Procedures

Endoscopic procedures of derivation population are detailed in our previous publication (Sacher-Huvelin S et al, Endoscopy 2015). In validation population #1, UGIE was performed by two senior endoscopists experienced in studies on PHT (Oberti F et al, Gastrointest Endosc 1998; 48:148-157). In this study, EV size was also classified qualitatively into 3 grades: 1: small, 2: medium or 3: large. Stages 2 and 3 were grouped in LEV (Pascal J P et al, N Engl J Med 1987; 317:856-861).

### Blood Tests and Elastometry

Blood tests—The following blood tests were calculated according to published or patented formulas. Hepascore (Adams L A et al, Clin Chem 2005; 51:1867-1873), Fib-4 (Sterling R K et al, Hepatology 2006; 43:1317-1325), APRI (Wai C T et al, Hepatology 2003; 38:518-526). FibroMeter^{VIRUS2G} (Leroy V et al, Clin Biochem 2008; 41:1368-1376), CirrhoMeter^{VIRUS2G} (Boursier J et al, Eur J Gastroenterol Hepatol 2009; 21:28-38), FibroMeter^{VIRUS3G} (Calès P et al, J Hepatol 2010; 52: S406) and CirrhoMeter^{VIRUS3G} (Calès P et al, J Hepatol 2010; 52: S406) were constructed for Metavir fibrosis staging in CHC. In FibroMeter/CirrhoMeter^{VIRUS3G} GGT replaces hyaluronate included in FibroMeter/CirrhoMeter^{VIRUS2G}. CirrhoMeter tests were constructed for cirrhosis diagnosis and included all FibroMeter markers ((Boursier J et al, Eur J Gastroenterol Hepatol 2009; 21:28-38). FibroMeter^{ALD} (Cales P et al, Gastroenterol Clin Biol 2008; 32:40-51) and FibroMeter^{NAFLD} (Cales P et al, J Hepatol 2009; 50:165-173) were constructed for Metavir fibrosis staging, respectively in alcoholic liver disease (ALD) and NAFLD. QuantiMeter^{NAFLD} was constructed to evaluate the area of whole fibrosis in NAFLD (Cales P et al, Liver Int 2010; 30:1346-1354). QuantiMeter^{VIRUS} and QuantiMeter^{ALD} were constructed to evaluate the area of whole fibrosis in CHC and ALD, respectively (Cales P, et al, Hepatology 2005; 42:1373-1381). All blood assays were performed in the same laboratories of each center, or partially centralized in population #3. Tests were used as raw data without correction rules like expert system.

Elastometry—Vibration control transient elastography (VCTE) (Fibroscan™, Echosens, Paris, France) examination was performed by an experienced observer (>50 examinations before the study), blinded for patient data. Examination conditions were those recommended by the manufacturer (Castera L et al, J Hepatol 2008; 48:835-847). VCTE examination was stopped when 10 valid measurements were recorded. Results (kilopascals) were expressed as the median and the interquartile range of all valid measurements.

Combined test—One test combined markers of blood test and VCTE: Elasto-FibroMeter^{2G} (E-FibroMeter^{2G}) (Cales P et al, Liver international: official journal of the International Association for the Study of the Liver 2014; 34:907-917).

### Statistics

### Diagnostic Test Segmentation

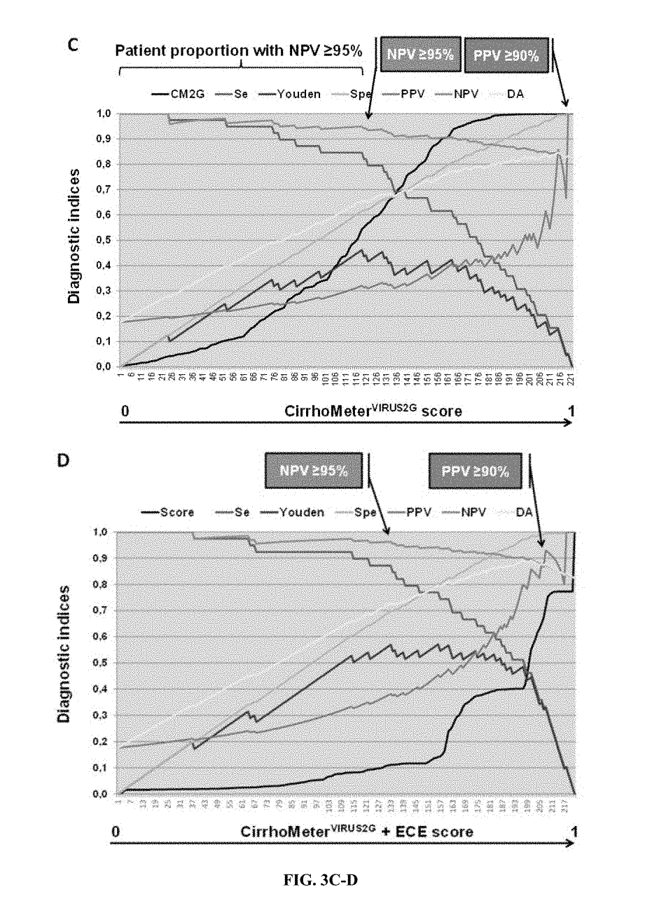

For LEV diagnosis, we calculated diagnostic indices as a function of test values (FIG. 3).

We determined the cut-off of test value to reach NPV≥95% and a PPV≥90% in the largest subpopulation when possible. PPV and NPV were reported through two statistical descriptors. First, the patient proportion (% out of the whole population) being included between the first test value reaching the expected predefined cut-off (95 or 90%) and the extreme test value, called PV patient proportion thereafter (see FIG. 3C). Second, the measured PV (%) was determined in this patient group. All test cut-offs for LEV were derived from derivation population and test accuracy was validated in validation populations by using the same cut-offs. Finally each test included three zones from the lowest to highest values: LEV exclusion, indeterminate, LEV affirmation.

### Clinical Descriptors

Ugie requirement—This is the patient proportion in the indeterminate zone between NPV and PPV cut-offs for LEV.

Missed LEV—This is the proportion of LEV in the NPV zone for LEV.

Saved UGIE—The reference patient group to calculate saved UGIE is the cirrhosis group where UGIE is classically performed: the whole population in derivation population and patients with Metavir F4 stage in validation population #1. The saved UGIE rate is the patient proportion provided by the difference between the reference group and the target group where UGIE is indicated by non-invasive tests. The target group can be determined by cut-offs of fibrosis staging or LEV diagnosis.

### Statistical Descriptors and Tests

Quantitative variables were expressed as mean±standard deviation. 95% confidence intervals (CI) were calculated by bootstrapping on 1000 samples. The discriminative ability of each test was expressed as the area under the receiver operating characteristic (AUROC) curve and compared by the Delong test. Data were reported according to STARD (Bossuyt P M et al, Clin Chem 2003; 49:7-189) and Liver FibroSTARD (Boursier J et al, J Hepatol 2015) statements, and analyzed on an intention to diagnose basis. Scores including independent predictors of LEV were determined by binary logistic regression. In the population where test is constructed, its accuracy is maximized and thus includes an optimism bias. Therefore, this bias was noticed when present. The main statistical analyses were performed under the control of professional statisticians using SPSS version 18.0 (IBM, Armonk, N.Y., USA) and SAS 9.2 (SAS Institute Inc., Cary, N.C., USA).

### Results

### Population Characteristics

Characteristics of main populations are described in table 2 and those of ancillary validation populations in table 3.

TABLE 2
Patient characteristics in the two main populations (with UGIE).

| | Population | | |
|---|---|---|---|
| Characteristic | Derivation | Validation #1 | |
| n patients | 287 | 165 | |
| Sex (M %) | 72.1 | 64.2 | |
| Age (yr) | 55.4 ± 10.7 | 50.1 ± 12.0 | |
| BMI (kg/m²) | 27.2 ± 5.6 | 24.0 ± 4.2 | |
| Cause (%): | | | |
| Alcohol | 64.5 | 72.7 | |
| Virus | 25.8 | 26.7 | |
| NAFLD | 5.6 | — | |
| Others | 4.2 | 0.6 | |
| Metavir F: | | | |
| 0 | 0 | 8.5 | |
| 1 | 0 | 19.4 | |
| 2 | 0 | 14.5 | |
| 3 | 0 | 6.7 | |
| 4 | 100^{a} | 50.9 | |
| Score | 4 | 2.7 ± 1.5 | |
| Child-Pugh class: | | | |
| A | 60.3 | 72.6 | (54.8)^{b} |
| B | 20.6 | 14.4 | (23.8) |
| C | 19.1 | 13.0 | (21.4) |
| Child-Pugh score: | 6.7 ± 2.5 | 6.3 ± 2.2 | (7.2 ± 2.5) |
| FibroMeter^{VIRUS2G} | 0.82 ± 0.21 | 0.74 ± 0.28 | (0.94 ± 0.10) |

| | | |
|---|---|---|
| Liver stiffness (kPa) | 33.4 ± 23.6 | — |
| Esophageal varices by | | |
| endoscopy/ECE (%): | | |
| No | 55.7/58.9 | 59.4 (29.8)/— |
| Small | 26.8/28.9 | 18.8 (27.4)/— |
| Large | 17.4/12.2 | 21.8 (42.9)/— |

BMI: body mass index,
ECE: esophageal capsule endoscopy,
NA: not available
^{a}Estimation
^{b}In cirrhosis in brackets

TABLE 3
Patient characteristics in the 3 ancillary validation populations.

| | Population | | |
|---|---|---|---|
| Characteristic | #2 | #3 | #4 |
| n patients | 1013 | 712 | 520 |
| Sex (M %) | 59.6 | 61.1 | 63.3 |
| Age (yr) | 45.4 ± 12.5 | 51.7 ± 11.2 | 54.5 ± 13.0 |
| Body mass index (kg/m²) | NA | 25.2 ± 4.6 | 29.6 ± 6.0 |
| Cause (%): | Virus | Virus | NAFLD |
| Metavir F stage: | | | |
| 0 | 4.3 | 3.8 | 23.3 |
| 1 | 43.3 | 37.8 | 31.5 |
| 2 | 27.0 | 25.7 | 19.2 |
| 3 | 13.9 | 17.8 | 16.3 |
| 4 | 11.4 | 14.9 | 9.6 |
| Score | 1.8 ± 1.1 | 2.0 ± 1.1 | 1.6 ± 1.3 |
| Child-Pugh class in F4: | A | A | A |
| FibroMeter^{VIRUS2G} | 0.50 ± 0.31 | 0.60 ± 0.28 | 0.48 ± 0.28 |
| Liver stiffness (kPa) | — | 10.0 ± 7.9 | 12.6 ± 11.3 |

NA: not available

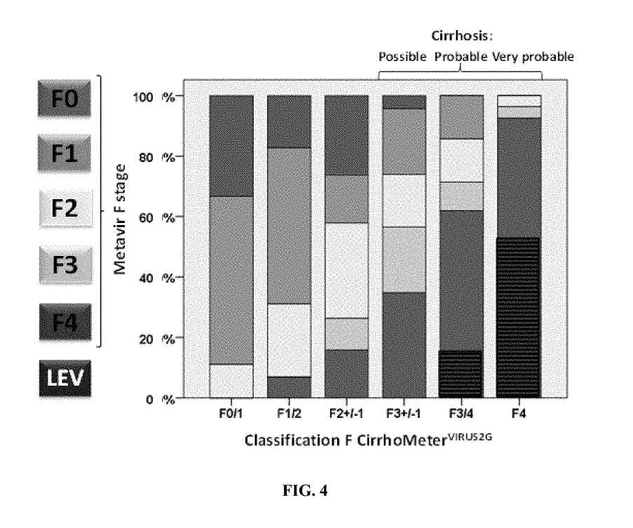

Differences between populations were observed with respect to etiology and severity of liver disease and also concerning the investigations performed (table 1). In validation CLD population #1, LEV were only observed in patients with confirmed cirrhosis according to liver biopsy and in those with probable cirrhosis according to CirrhoMeter^{VIRUS2G} (FIG. 4).

### Overall Accuracy of Tests

Accuracies by AUROC of predictors for LEV are detailed in table 4.

TABLE 4
AUROC for large esophageal varices. Markers are ranked by increasing
order in the derivation population with common size. Italicized
entries distinguish 0.1 intervals in AUROC.

| | | Population | | | | |
|---|---|---|---|---|---|---|
| | | Derivation | | | | |
| | | Maximum size | | | Common | |
| | | N | | | size | Validation |
| Marker | | patients | AUROC (95% CI) | | AUROC ^{a} | #1 ^{b} |
| 1. | Age | 287 | 0.501 | (0.414-0.589) | 0.443 | 0.707 |
| 2. | Spleen diameter | 119 | 0.518 | (0.400-0.637) | 0.508 | 0.697 |
| 3. | ALT | 287 | 0.532 | (0.445-0.619) | 0.516 | 0.705 |
| 4. | Leucocytes | 283 | 0.527 | (0.436-0.617) | 0.522 | — |
| 5. | Body mass index | 270 | 0.479 | (0.396-0.561) | 0.526 | 0.630 |
| 6. | GGT | 287 | 0.582 | (0.500-0.664) | 0.526 | 0.562 |
| 7. | Alpha2-macroglobulin | 248 | 0.524 | (0.436-0.612) | 0.529 | 0.656 |
| 8. | Weight | 278 | 0.491 | (0.414-0.567) | 0.531 | 0.592 |
| 9. | Segmented leucocytes | 216 | 0.578 | (0.476-0.679) | 0.534 | — |
| 10. | Height | 273 | 0.568 | (0.484-0.652) | 0.563 | 0.403 |
| 11. | Monocytes | 216 | 0.565 | (0.459-0.671) | 0.571 | — |
| 12. | Hemoglobin | 284 | 0.661 | (0.578-0.744) | 0.629 | 0.654 |
| 13. | P2/MS | 216 | 0.619 | (0.515-0.722) | 0.632 | — |
| 14. | Alphafoeto protein | 261 | 0.595 | (0.510-0.680) | 0.646 | — |
| 15. | Alkaline phosphatases | 282 | 0.652 | (0.578-0.726) | 0.647 | — |
| 16. | Sodium | 263 | 0.639 | (0.557-0.721) | 0.674 | 0.521 |
| 17. | Platelets | 284 | 0.630 | (0.536-0.725) | 0.675 | 0.769 |
| 18. | AST | 287 | 0.646 | (0.570-0.721) | 0.681 | 0.494 |
| 19. | InflaMeter | 246 | 0.642 | (0.556-0.727) | 0.684 | — |
| 20. | Creatinine | 283 | 0.610 | (0.525-0.694) | 0.686 | 0.523 |
| 21. | Urea | 279 | 0.681 | (0.594-0.767) | 0.698 | 0.536 |
| 22. | APRI | 284 | 0.655 | (0.576-0.733) | 0.704 | 0.682 |
| 23. | Child-Pugh score | 287 | 0.718 | (0.640-0.796) | 0.718 | 0.782 |
| 24. | Fib-4 | 284 | 0.702 | (0.625-0778) | 0.725 | 0.790 |
| 25. | VCTE | 211 | 0.738 | (0.662-0.815) | 0.730 | — |
| 26. | Albumin | 275 | 0.727 | (0.655-0.799) | 0.734 | 0.743 |
| 27. | FibroMeter for cause | 251 | 0.736 | (0.667-0.805) | 0.736 | — |
| 28. | AST/ALT | 287 | 0.737 | (0.667-0.807) | 0.747 | 0.678 |
| 29. | Prothrombin index | 284 | 0.733 | (0.660-0.807) | 0.752 | [symbol] |
| 30. | FibroMeter^{VIRUS3G} | 243 | 0.755 | (0.680-0.829) | 0.761 | [symbol] |
| 31. | CirrhoMeter^{VIRUS3G} | 243 | 0.752 | (0.678-0.827) | 0.763 | [symbol] |
| 32. | Bilirubin | 284 | 0.738 | (0.670-0.806) | 0.771 | [symbol] |
| 33. | Elasto-FibroMeter^{VIRUS2G} | 160 | 0.775 | (0.694-0.857) | 0.773 | — |
| 34. | AST/ALT + prothrombin | 284 | 0.763 | (0.693-0.834) | 0.778 | [symbol] |
| 35. | Hyaluronate | 225 | 0.772 | (0.703-0.842) | 0.794 | [symbol] |
| 36. | AST/ALT + hyaluronate | 225 | 0.777 | (0.702-0.853) | 0.794 | [symbol] |
| 37. | QuantiMeter^{VIRUS} | 210 | 0.707 | (0.618-0.795) | 0.799 | [symbol] |
| 38. | QuantiMeter for cause | 249 | 0.770 | (0.701-0.839) | 0.799 | — |
| 39. | CirrhoMeter^{VIRUS2G} | 211 | 0.765 | (0.683-0.847) | 0.800 | 0.911 |
| 40. | Hepascore | 213 | 0.768 | (0.693-0.842) | 0.801 | 0.863 |
| 41. | FibroMeter^{VIRUS2G} | 211 | 0.768 | (0.686-0.850) | 0.810 | 0.884 |
| 42. | EV stage by ECE (15 mm) | 287 | 0.874 | (0.819-0.929) | 0.845 | — |
| 43. | EV stage by ECE (25 mm) | 287 | 0.885 | (0.829-0.840) | 0.867 | — |

| | | | | | |
|---|---|---|---|---|---|
| 44. | ECE + CirrhoMeter^{VIRUS2G} | 211 | [symbol] | [symbol] | — |
| 45. | ECE + AST/ALT | 287 | [symbol] | [symbol] | — |

ECE: esophageal capsule endoscopy,
EV: esophageal varices,
VCTE: vibration control transient elastography.
AUROCs > 0.8 are shown in bold
^{a} 158 patients.
^{b} Validation population #1 including 165 patients

Briefly, in derivation population, the highest AUROC at 0.92 was obtained with ECE+(AST/ALT) score. This score and ECE+CirrhoMeter^{VIRUS2G} score had significantly higher AUROC than fibrosis tests (p<0.02) whereas AUROCs between other fibrosis tests were not significantly different (data not shown). In validation population, AUROC of Metavir F stage was significantly inferior to that of the most accurate tests (CirrhoMeters and FibroMeter^{VIRUSS2G}). This suggests that non-invasive testing can be more effective for LEV diagnosis than a histological diagnosis of cirrhosis.

### Diagnostic Strategies

### Strategy Selection According to Accuracy

### LEV Absence

Derivation population—CirrhoMeter^{VIRUS2G} was the most accurate low constraint test resulting in the highest NPV patient proportion and the highest measured NPV for LEV among all strategies (table 5).

TABLE 5
Different diagnostic strategies for LEV developed in derivation population (with common size: n =
158) according to high negative (absence) and positive (presence) predictive value zones for LEV.
The indeterminate zone lies between the two previous zones and corresponds to endoscopy requirement.
These figures are proportions among all patients. The rate of missed LEV is the proportion of patients
with LEV in the absence zone among patients with LEV. Figures in brackets are 95% CI.

| | Large EV | | | | | | | |
|---|---|---|---|---|---|---|---|---|
| Strategy | Absence | | Indeterminate | | Presence | | Missed | |
| Single test: | | | | | | | | |
| ECE | | | | | | | | |
| Patients (%) | 59.5 | (51.6-67/5) | 29.1 ^{a} | (21.5-36.1) | 11.4 | (6.9-16.8) | 10.0 | (0-22.2) |
| Predictive value (%) | 96.8 | (92.9-100) | — | | 83.3 ^{b} | (62.5-100) | — | |
| VCTE | | | | | | | | |
| Patients (%) | 47.5 | (40.0-55.7) | 52.5 | (44.4-60.1) | 0 ^{c} | (0-0) | 13.3 | (2.7-26.1) |
| Predictive value (%) | 94.7 | (88.6-98.7) | — | | — | | | |
| (AST/ALT) + PI score | | | | | | | | |
| Patients (%) | 55.1 | (46.7-62.3) | 44.3 | (36.7-51.9) | 0.6 | (0-2.0) | 16.7 | (3.7-30.8) |
| Predictive value (%) | 94.3 | (89.3-98.8) | — | | 100 | (100-100) | — | |
| (AST/ALT) + hyaluronate | | | | | | | | |
| score | | | | | | | | |
| Patients (%) | 54.4 | (46.5-62.0) | 44.3 | (35.5-51.9) | 1.3 | (0-3.2) | 20.0 | (6.1-35.1) |
| Predictive value (%) | 93.0 | (87.5-97.9) | — | | 100 | (100-100) | — | |
| CirrhoMeter^{VIRUS2G} | | | | | | | | |
| Patients (%) | 55.7 | (47.7-63.1) | 40.5 | (33.1-48.1) | 3.8 | (1.2-7.2) | 13.3 | (3.0-27.3) |
| Predictive value (%) | 95.5 | (90.4-99.0) | — | | 83.3 | (NA) | — | |
| Simultaneous | | | | | | | | |
| combination: | | | | | | | | |
| ECE + (AST/ALT) score | | | | | | | | |
| Patients (%) | 78.5 | (72.1-84.8) | 11.4 | (6.3-16.5) | 10.1 | (5.7-15.1) | 26.7 | (11.5-44.1) |
| Predictive value (%) | 93.5 | (88.9-97.6) | — | | 93.8 | (76.9-100) | — | |
| ECE + CirrhoMeter^{VIRUS2G} | | | | | | | | |
| score | | | | | | | | |
| Patients (%) | 58.9 | (50.3-66.2) | 34.8 | (27.6-42.6) | 6.3 | (3.0-11.0) | 6.7 | (0.0-16.7) |
| Predictive value (%) | 97.8 | (94.7-100) | — | | 100 | (100-100) | — | |
| Sequential combination: | | | | | | | | |
| VCTE/ECE | | | | | | | | |
| Patients (%) | 47.5 | (40.0-55.7) | 43.0 | (35.4-51.3) | 9.5 ^{d} | (5.1-14.2) | 13.3 | (2.7-26.1) |
| Predictive value (%) | 94.7 | (88.7-98.7) | — | | 93.3 | (76.9-100) | — | |
| VCTE/ECE + (AST/ALT) | | | | | | | | |
| score | | | | | | | | |
| Patients (%) | 47.5 | (40.0-55.7) | 43.7 | (36.1-51.9) | 8.9 ^{e} | (4.7-13.5) | 13.3 | (2.7-26.1) |
| Predictive value (%) | 94.7 | (89.0-98.8) | — | | 92.9 | (76.9-100) | — | |
| CirrhoMeter^{VIRUS2G}/ | | | | | | | | |
| ECE + CirrhoMeter^{VIRUS2G} | | | | | | | | |
| score | | | | | | | | |
| Patients (%) | 65.8 | (57.6-73.1) | 26.6 | (19.7-33.5) | 7.6 | (3.7-12.4) | 13.3 | (3.0-27.3) |

| | | | | | | |
|---|---|---|---|---|---|---|
| Predictive value (%) | 96.2 | (92.0-99.1) | — | 91.7 | (71.4-100) | — |

| | | | | |
|---|---|---|---|---|
| p ^{f} | <0.001 | <0.001 | <0.001 | 0.648 |

LEV: large esophageal varices,
ECE: esophageal capsule endoscopy,
PI: prothrombin index
VCTE: vibration control transient elastography.
Best results are shown in bold and worst in italics per zone
^{a} Patients diagnosed with small EV by ECE
^{b} It was not possible to reach the objective (≥90%) since this is semi-quantitative variable.
^{c} Maximum PPV was <40%
^{d} This figure is different from ECE alone since 3 patients with high LEV PPV with ECE had high LEV NPV with VCTE
^{e} This figure is different from ECE + AST/ALT score alone (simultaneous combination) since 2 patients with high LEV PPV with ECE + AST/ALT score had high LEV NPV with VCTE
^{f} By paired Cochran test for patient proportions. Useful pairwise comparisons are provided in the text

In the largest sample size tested for CirrhoMeter^{VIRUS2G} (table 6) the measured PPV was 98% (95% CI: 95-100) in a patient proportion of 59% (53-66).

TABLE 6
Comparison of LEV prediction between all 4 strategies based on esophageal
capsule endoscopy (ECE) and/or CirrhoMeter^{VIRUS2G} (CM). Figures in
brackets are 95% CI. Derivation population (211 patients).

| | Large EV | | | | |
|---|---|---|---|---|---|
| Strategy | Absence | | Indeterminate | Presence | |
| 1. ECE | | | | | |
| Patients (%) | 58.8 | (52.6-65.7) | 29.4 (23.3-35.5) | 11.8 | (7.6-16.5) |
| Predictive value (%) | 97.6 | (94.9-100) | — | 80.0 | (61.9-95.0 |
| 2. CirrhoMeter^{VIRUS2G} | | | | | |
| Patients (%) | 53.1 | (46.7-60.1) | 44.1 (37.1-50.7) | 2.8 | (0.9-5.2) |
| Predictive value (%) | 94.6 | (89.9-98.3) | — | 83.3 | (NA) |
| 3. ECE + CirrhoMeter^{VIRUS2G} | | | | | |
| score | | | | | |
| Patients (%) | 58.3 | (52.2-64.9) | 35.1 (28.5-40.9) | 6.6 | (3.3-10.3) |
| Predictive value (%) | 98.4 | (95.6-100) | — | 92.9 | (75.0-100) |
| 4. CirrhoMeter^{VIRUS2G}/ECE + | | | | | |
| CirrhoMeter^{VIRUS2G} score | | | | | |
| Patients (%) | 64.5 | (57.9-71.1) | 28.0 (22.1-34.2) | 7.6 | (4.1-11.5) |
| Predictive value (%) | 95.6 | (92.1-98.5) | — | 87.5 | (68.8-100) |
| Comparison ^{a} | | | | | |

| | | | |
|---|---|---|---|
| All 4 strategies | 0.001 | <0.001 | <0.001 |
| ECE vs CM | 0.188 | 0.001 | <0.001 |
| ECE vs ECE + CM | 1 | 0.096 | 0.001 |
| ECE vs CM/ECE + CM | 0.104 | 0.775 | 0.022 |
| CM vs ECE + CM | 0.099 | 0.009 | 0.039 |
| CM vs CM/ECE + CM | <0.001 | <0.001 | 0.002 |
| ECE + CM vs CM/ECE + CM | <0.001 | <0.001 | 0.500 |

NA: not available
^{a} Between patients proportions by paired Cochran test between the 4 strategies or paired McNemar test for pairwise comparison

Validation populations—With regard to the low constraint tests in validation population #1 (table 7), the most performant was again CirrhoMeter^{VIRUSV2G} since providing the highest measured NPV for LEV—99% (97-100)—in the highest NPV patient proportion—60% (53-68)—(table 8).

TABLE 7
Rates (%) of LEV prediction by LEV cut-offs of blood tests -from
derivation population applied to validation population #1- according
to LEV or cirrhosis presence. Figures in brackets are 95% CI.

| | Large EV | | | | |
|---|---|---|---|---|---|
| Blood test | Absence | | Indeterminate ( [symbol] ) | Presence | |
| (AST/ALT) + PI score: | | | | | |
| Predictive value ^{a} | 92.8 | | — | 100 | |
| Patient proportion: | | | | | |
| No LEV | 69.8 | ( [symbol] ) | 30.2 | 0 | ( [symbol] ) |
| LEV | 19.4 ^{b} | ( [symbol] ) | 77.8 | 2.8 | ( [symbol] ) |
| F0-3 | 91.4 | ( [symbol] ) | 8.6 | 0 | ( [symbol] ) |
| F4 | 27.4 | | 71.4 | 1.2 | ( [symbol] ) |
| All patients | 58.8 | (51.3-66.3) | 40.6 (33.0-48.2) | 0.6 | (0-1.9) |
| (AST/ALT) + hyaluronate | | | | | |
| score: | | | | | |
| Predictive value ^{a} | 97.6 | | — | 66.7 | |
| Patient proportion: | | | | | |
| No LEV | 64.3 | ( [symbol] ) | 34.9 | 0.8 | ( [symbol] ) |
| LEV | 5.6 ^{b} | ( [symbol] ) | 88.9 | 5.6 | ( [symbol] ) |
| F0-3 | 85.2 | ( [symbol] ) | 14.8 | 0 | ( [symbol] ) |
| F4 | 19.0 | | 77.4 | 3.6 | ( [symbol] ) |
| All patients | 51.5 | (43.9-59.0) | 46.7 (39.3-54.4) | 1.8 | (0-4.2) |
| CirrhoMeter^{VIRUS2G}: | | | | | |
| Predictive value ^{a} | 98.9 | | — | — | |
| Patient proportion: | | | | | |
| No LEV | 74.2 | ( [symbol] ) | 25.8 | 0 | ( [symbol] ) |
| LEV | 3.2 ^{b} | ( [symbol] ) | 96.8 | 0 | ( [symbol] ) |
| F0-3 | 92.4 | ( [symbol] ) | 7.6 | 0 | ( [symbol] ) |
| F4 | 26.3 | | 73.7 | 0 | ( [symbol] ) |
| All patients | 60.0 | (52.6-67.7) | 40.0 (32.3-47.4) | 0 | (0-0) |
| Comparison ^{c} | | | | | |

| | | | |
|---|---|---|---|
| Missed LEV | p = 0.007 | — | — |
| UGIE requirement | — | p = 0.030 | — |

LEV: large esophageal varices,
PI: prothrombin index,
F: Metavir fibrosis stage,
UGIE: upper gastro-intestinal endoscopy.
Best results are shown in bold and worst in italics per zone and patient category or predictive value.
Arrows indicate the clinically suitable trends, e.g. LEV exclusion zone should be very low in no LEV or F0-3 patients and LEV affirmation zone high in LEV or F4 patients
^{a} Measured predictive value in all patients
^{b} Corresponds to missed LEV
^{c} By paired Cochran test between the 3 tests

TABLE 8
Rates (%) of LEV prediction by CirrhoMeter^{VIRUS2G} cut-offs for
LEV -from derivation population- according to LEV or cirrhosis
presence in CLD validation population #1. Figures in brackets
are 95% CI. This table details some results of table 7.

| | Large EV | | | | |
|---|---|---|---|---|---|
| Blood test | Absence | | Indeterminate ( [symbol] ) | | Presence |
| Predictive value: | | | | | |
| All patients | 98.9 | (96.6-100) | — | | — |
| No LEV | 100 | (100-100) | — | | — |
| LEV | 0 | | — | | — |
| F0-3 | 100 | (100-100) | — | | — |
| F4 | 59.2 | (47.2-70.5) | — | | — |
| Patient proportion: | | | | | |
| All patients | 60.0 | (52.6-67.7) | 40.0 | (32.3-47.4) | 0 |
| No LEV | 74.2 | (67.4-81.5) ( [symbol] ) | 25.8 | (18.5-32.6) | 0 ( [symbol] ) |
| LEV | 3.2 | (0.0-10.3) ( [symbol] ) ^{a} | 96.8 | (89.7-100) | 0 ( [symbol] ) |
| F0-3 | 92.4 | (86.3-98.6) ( [symbol] ) | 7.6 | (1.4-13.8) | 0 ( [symbol] ) |
| F4 | 26.3 | (17.2-37.0) | 73.7 | (63.0-82.8) | 0 ( [symbol] ) |

LEV: large esophageal varices,
PI: prothrombin index,
F: Metavir fibrosis stage,
UGIE: upper gastro-intestinal endoscopy.
Arrows indicate the clinically suitable trends, e.g. LEV exclusion zone should be very low in no LEV or F0-3 patients and LEV affirmation zone high in LEV or F4 patients
^{a} Corresponds to missed LEV

In validation populations #2 to #4 (2245 CLD patients: table 9), CirrhoMeter^{VIRUS2G} was the test with the highest NPV patient proportion (298%) in non-cirrhotic patients across the 3 populations.

TABLE 9
Robustness of cut-offs of blood tests for predictive values
for LEV, as determined in the derivation population, in validation
populations #2 to #4 (2245 CLD patients): patient
proportion (%) as a function of cirrhosis (F4) presence. Results
of validation population #1 are grouped in table 8.

| | Validation population | | | | | | |
|---|---|---|---|---|---|---|---|
| | #2 ^{a} | | #3 ^{a} | | #4 | | |
| Fibrosis test | NPV | Indet. | NPV | Indet. | NPV | Indet. | PPV |

AST/ALT + PI score:

| | | | | | | | |
|---|---|---|---|---|---|---|---|
| F0-F3 | 98.9 | 1.1 | 98.5 | 1.5 | 96.6 | 3.4 | 0 |
| F4 | 77.4 | 22.6 | 91.5 | 8.5 | 74.0 | 26.0 | 0 |

AST/ALT + hyaluronate score:

| | | | | | | | |
|---|---|---|---|---|---|---|---|
| F0-F3 | 97.3 | 2.7 | 95.2 | 4.8 | 90.9 | 9.1 | 0 |
| F4 | 55.7 | 44.3 | 68.9 | 31.1 | 56.0 | 42.0 | 2.0 |

CirrhoMeter^{VIRUS2G}:

| | | | | | | | |
|---|---|---|---|---|---|---|---|
| F0-F3 | 98.7 | 1.3 | 98.3 | 1.7 | 98.6 | 1.4 | 0 |
| F4 | 55.3 | 46.1 | 68.9 | 31.1 | 68.3 | 31.7 | 0 |

VCTE:

| | | | | | | | |
|---|---|---|---|---|---|---|---|
| F0-F3 | — | — | 99.0 | 1.0 | 94.1 | 5.9 | 0 |
| F4 | — | — | 74.0 | 36.0 | 45.5 | 54.5 | 0 |

Indet.: indeterminate zone between NPV and PPV zones;
NPV: negative predictive value zone,
PPV: positive predictive value zone,
PI: prothrombin index,
VCTE: vibration control transient elastography.
Clinically satisfactory results are shown in bold and those unsatisfactory are in italics
^{a} No PPV zone (0% patients)

### LEV Presence

Derivation population—Among the five single test strategies (table 5), ECE was the most accurate test due to a significantly lower indeterminate patient proportion (29%, p<0.001) and the highest PPV patient proportion (11%). However, the measured PPV for LEV in this subgroup did not reach the targeted value: 80% in the largest population (table 6). Among the five combination strategies, two strategies ranked first for the two PPV criteria. Thus, ECE+(AST/ALT) score had the highest PPV patient proportion (10%) but was hampered by a suboptimal measured PPV—94% (77-100)—for LEV despite optimism bias. ECE+CirrhoMeter^{VIRUSS2G} score reached the highest measured PPV for LEV at the expense of a lower PPV patient proportion than in other combinations (table 5). Measured PPV was 93% (75-100) in a patient proportion of 7% (3-10) in the largest population (table 6).

Validation populations—In validation population #1 (table 7), the three available blood tests showed similar results to derivation population (i.e. a high PPV was only observed in 1% of patients despite a 22% LEV prevalence). Thus, blood tests alone are not sufficiently predictive of the presence of LEV.

### Most Accurate Strategy

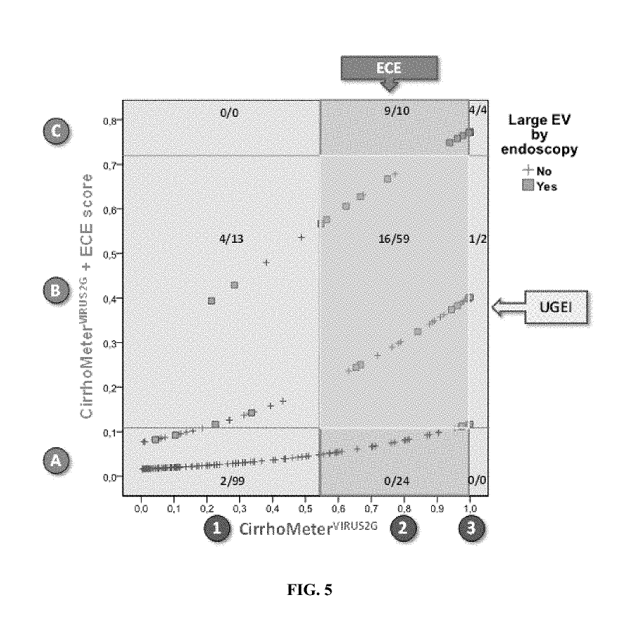

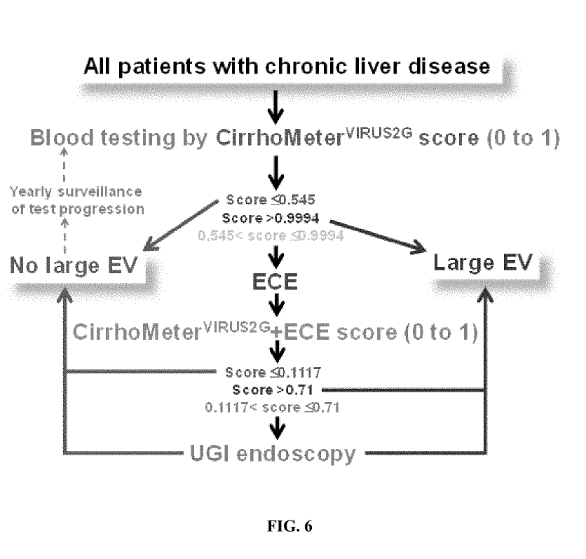

Among several clinically applicable strategies, the most accurate one seemed at this step to use first CirrhoMeter^{VIRUS2G} mainly for LEV absence (i.e. the test with the highest NPV criteria), then to use the ECE+CirrhoMeter^{VIRUS2G} score for LEV presence (i.e. a 93% PPV value in a substantial patient proportion). As there was a significant interaction (p<0.001) between CirrhoMeter^{VIRUS2G} and ECE, we analyzed the plot of the two tests (FIG. 5). It clearly shows that UGIE has only to be required in the indeterminate zone common to the two tests which resulted in the proposed sequential diagnostic algorithm shown in FIG. 6 and called VariScreen algorithm thereafter.

### Test Robustness

Robustness of test cut-off values for LEV prediction was evaluated in large unselected validation populations #2 to #4 (2245 CLD patients without decompensated cirrhosis) (table 9). Briefly, CirrhoMeter^{VIRUS2G} robustness was validated, especially its estimated specificity was 100%, i.e. there was a priori no false positive LEV prediction in non-cirrhotic patients like in validation population #1.

### Strategy Evaluation According to Clinical Aspects

### LEV Strategy Comparison

All strategies were compared within the derivation population (table 5). Among the 7 clinically applicable strategies, the most accurate were the 3 combined sequential strategies since the two clinical descriptors (UGIE requirement and missed LEV) were better or equal than in the 4 single test strategies. Among these 3 combined sequential strategies, that including CirrhoMeter^{VIRUS2G} offered the advantage of a higher rate of saved ECE (i.e. NPV patient proportion) or UGIE than the two others (p<0.001) with similar missed EV rate. Finally, the two strategies combining CirrhoMeter^{VIRUS2G} and ECE (simultaneous or sequential) were directly compared (tables 6 and 10): the simultaneous combination resulted in significant lower missed LEV rate. However, the sequential strategy significantly reduced UGIE requirement (−7%) while saving 65% ECE.

TABLE 10
Rates (%) of saved endoscopy and missed LEV by using two strategies based
on CirrhoMeter^{VIRUS2G} ± ECE. The first strategy (A) is the recent
attitude of performing UGIE according to non-invasive fibrosis staging;
the second strategy (B) is that developed in the present study based on
non-invasive tests targeted for LEV. Figures in brackets are 95% CI.

| | Saved | | | |
|---|---|---|---|---|
| Strategy | endoscopy | | Missed LEV | |
| Derivation population ^{a}: | | | | |
| A. Cirrhosis staging by CM ^{b} followed by UGIE in: | | | | |
| Possible cirrhosis | 15.6 | (10.7-20.5) | 2.8 | (0.0-9.1) |
| Probable cirrhosis | 36.5 | (30.2-43.0) | 11.1 | (2.3-21.4) |
| Very probable cirrhosis | 61.1 | (54.5-67.4) | 33.3 | (18.2-50.0) |
| p ^{c} | <0.001 | | <0.001 | |
| B. LEV prediction according to ^{d}: | | | | |
| CM | 55.9 | (49.5-62.8) | 16.7 | (4.6-29.4) |
| CM + ECE score | 64.9 | (58.3-71.6) | 5.6 | (0.0-14.3) |
| CM/CM + ECE score | 72.0 | (65.7-78.1) | 16.7 | (4.6-29.4) |
| p ^{e} | <0.001 | | 0.018 | |
| Validation population #1: | | | | |
| A. Cirrhosis staging by CM ^{b} followed by UGIE in: | | | | |
| Possible cirrhosis | −28.9 | (p < 0.001) ^{f} | 0 | |
| Probable cirrhosis | 1.3 | (p = 1) ^{f} | 0 | |
| Very probable cirrhosis | 28.9 | (p < 0.001) ^{f} | 9.7 | (0.0-21.9) |
| p ^{g} | <0.001 | | 0.048 | |
| B. LEV prediction according to ^{d h}: | | | | |
| CM | 18.4 | (p = 0.009) ^{f} | 3.2 | (0.0-10.3) |
| CM/CM + ECE score | 48.2 ⁱ | (p < 0.001) ^{f} | 3.2 | (0.0-10.3) |

| | | |
|---|---|---|
| p ^{k} | <0.001 | 1 |

ECE: esophageal capsule endoscopy,
CM: CirrhoMeter^{VIRUS2G},
LEV: large esophageal varices,
F: Metavir fibrosis stage.
Satisfactory results are shown in bold and unsatisfactory in italics per population and strategy
^{a} The rates were calculated in the derivation population with maximum size (n = 211)
^{b} Cut-offs of CM classes are those defined a priori for fibrosis stages in previous publication (Cales P et al, Journal of clinical gastroenterology 2014): cirrhosis is defined as possible (classes F3 ± 1, F3/4 and F4) or probable (classes F3/4 and F4) or very probable (classes F4). UGIE is performed only in the classes selected. The significance of gain could not be calculated since UGIE was performed in every patient
^{c} By paired Cochran test between the 3 proportions. Pairwise comparisons by paired McNemar test: saved UGIE: all three pairs: p < 0.001; missed LEV: possible vs probable: p = 0.250, possible vs very probable: p = 0.001, probable vs very probable: p = 0.008
^{d} Cut-offs of CirrhoMeter^{VIRUS2G} classes and scores were defined a posteriori for LEV in the current derivation population. ECE is performed outside the PV zones of CM and UGIE is performed in the indeterminate zone
^{e} By paired Cochran test between the 3 proportions. Pairwise comparisons by McNemar test for saved UGIE: CM vs CM/CM + ECE: p = 0.001, CM vs CM + ECE: p < 0.001, CM/CM + ECE vs CM + ECE: p = 0.038
^{f} Comparison vs UGIE in histological cirrhosis by paired McNemar test. 95% CI cannot be calculated since this figure is obtained by a proportion difference
^{g} By paired Cochran test between the 3 proportions. Pairwise comparisons by McNemar test: saved UGIE: all three pairs: p < 0.001; missed LEV: not calculable
^{h} The simultaneous strategy based on CirrhoMeter^{VIRUS2G} + ECE score was not evaluated since clinically unsuitable in a CLD population
ⁱ Estimated calculation by applying the rate of saved UGIE by CM + ECE score in the indeterminate CM zone from derivation population (36.6%)
^{k} By paired McNemar test

### Comparison of Direct LEV Screening Vs Indirect Fibrosis Staging

We compared the direct LEV screening developed in the present study vs an indirect screening based on cirrhosis diagnosis (reference for UGIErequirement in the present study) or non-invasive fibrosis staging. Thus, we compared the three strategies including CirrhoMeter^{VIRUS2G} (table 10). Briefly, in the validation CLD population, a strategy of performing endoscopy by the possible cirrhosis class of CirrhoMeter^{VIRUS2G} would induce a significant UGIE overuse of 29% compared to conventional histological diagnosis of cirrhosis. Finally, at similar missed LEV rate, the saved UGIErate was much higher when CirrhoMeter^{VIRUS2G} was targeted for LEV than for fibrosis, e.g. 18.4% (p=0.009) vs. 1.3% (p=1), respectively in derivation population.

### Clinical Improvement by Sequential Combination of CirrhoMeter^{VIRUS2G} to ECE

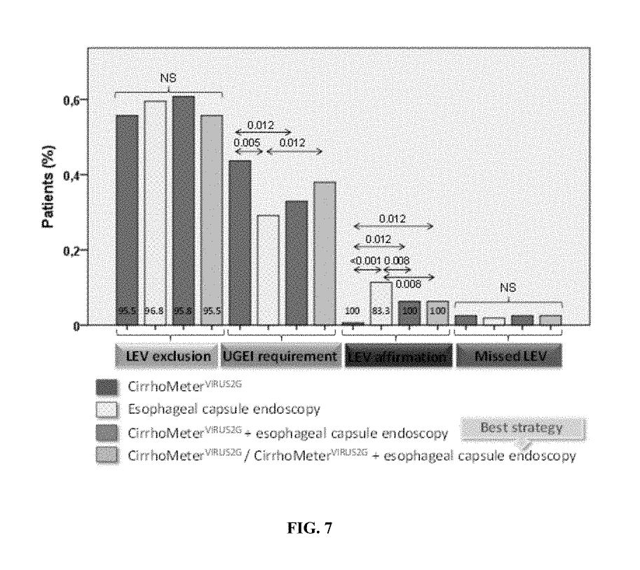

Direct comparison of CirrhoMeter^{VIRUS2G}, ECE and their combinations was performed in derivation population (FIG. 7, table 6).

CirrhoMeter^{VIRUS2G} was as accurate as ECE to predict LEV absence. However, ECE was significantly more accurate than CirrhoMeter^{VIRUS2G} to predict LEV presence. Sequential combination significantly decreased the patient proportion with LEV presence from 12 to 8% compared to simultaneous combination but this was counterbalanced by an increase in measured PPV from 83% to 88%. The UGIE requirement by this sequential combination was significantly reduced when compared to CirrhoMeter^{VIRUS2G} but not significantly different compared to ECE. The missed EV rate was significantly decreased by simultaneous combination compared to other strategies only in the derivation population. Finally, the sequential combination spared 48 to 72% of UGIE, whether UGIE would have been performed in all patients with cirrhosis, and spared 65% of ECE, whether ECE would have been performed in all CLD. The rate of correctly classified patients for LEV was, CirrhoMeter. 96.7% ((94.3-99.0), ECE: 90.0% (86.1-93.9), (CirrhoMeter+ECE) score: 98.6% (96.7-100), VariScreen algorithm: 96.2% (93.1-98.6), p<0.001 by paired Friedman test (pairwise comparison: ECE significantly lower than other tests and other test not significantly different between each other).

### Misclassified Patients

They were 21 patients (10.0%) misclassified for LEV by ECE; 5 were false positive LEV by ECE and 4 were rescued by VariScreen; there were 16 false negative LEV by ECE and 15 were rescued by VariScreen. Thus, 20 patients were rescued. However, there were 6 false negative LEV and 1 false positive LEV by VariScreen so that the net result was 20−7=13 corresponding to the 6.2% gain in accuracy by VariScreen compared to ECE. The 6 patients with missed LEV by VariScreen algorithm had blood markers significantly different (reflecting a better liver status) from other patients with LEV, e.g. serum albumin level (not included in CirrhoMeter): ruling out zone: 36.2±6.9 g/l, indeterminate zone: 34.1±5.8, ruling in zone: 25.9±5.1, p<0.001 by ANOVA.

### Discussion

### Originalities

The present study is the first one to compare ECE and fibrosis tests for the non-invasive diagnosis of LEV (Colli A et al, Cochrane Database Syst Rev 2014; 10:CD008760). In addition, studies of non-invasive diagnosis of LEV were performed in patients with cirrhosis selected by other means than non-invasive fibrosis tests. This casts some uncertainty about the cut-off exportability of non-invasive test cut-offs for LEV diagnosis in populations where cirrhosis will be diagnosed by the same non-invasive tests (for fibrosis staging there) among CLD. Therefore, we also evaluated together these tests in a population of CLD including non-cirrhotic patients with available UGIE which is a unique population. The only diagnostic combination algorithm published for high-risk EV was a sequential algorithm based on liver stiffness and concordant blood test in a first step followed by spleen stiffness in the intermediate zone; but the accuracy was only around 77% (Stefanescu H et al, Liver Int 2014).

### Main Results

Among fibrosis tests, blood tests appeared more interesting than VCTE for LEV diagnosis. This is due not only to a low maximum PPV for LEV but also to a lesser NPV patient proportion with VCTE (table 6). VCTE has been shown to well diagnose PHT level (Bureau C et al, Aliment Pharmacol Ther 2008; 27:1261-1268) but was limited and inferior to a single blood marker, like prothrombin index, for LEV diagnosis (Castera L et al, J Hepatol 2009; 50:59-68).

Among single tests, ECE was the most accurate non-invasive diagnosis for LEV providing the lowest rates of endoscopy requirement and missed LEV (table 5). In clinical practice, we have to choose a sequential diagnostic strategy based on a low constraint test to exclude LEV (expectedly in non-cirrhotic CLD) and on the most accurate test to diagnose LEV (expectedly used in cirrhosis). The most accurate sequential strategy was the VariScreen algorithm (FIG. 6) both in validation and derivation populations with a rate of saved endoscopy of 48 to 72% and a rate of missed LEV of 3 to 17%, respectively. In practical terms, CirrhoMeter^{VIRUS2G} is performed in all CLD patients. Patients with CirrhoMeter^{VIRUS2G} below NPV LEV cut-off are followed-up with yearly testing. Those with CirrhoMeter^{VIRUS2G} beyond PPV LEV cut-off are offered primary prophylaxis. Those between the two CirrhoMeter^{VIRUS2G} LEV cut-offs are offered ECE. Then, the ECE+CirrhoMeter^{VIRUS2G} score is calculated by computerization. Patients with a score below NPV LEV cut-off are followed-up with yearly CirrhoMeter^{VIRUS2G} testing. Patients with a score beyond PPV LEV cut-off are offered primary prophylaxis, either pharmacological or endoscopic (which could be a preferable option to validate non-invasive diagnosis in rare cases without LEV on ECE).

Patients between the two LEV cut-offs of ECE+CirrhoMeter^{VIRUS2G} score are offered UGIE (FIG. 6).

Finally, this study confirms that the non-invasive cirrhosis diagnosis has the potential to induce a roughly 30% endoscopy overuse. But applying cut-off of single fibrosis test specific for LEV is able to save one out 5 endoscopies compared to conventional strategy with cirrhosis diagnosis determined by liver biopsy (table 10).

### Result Comments

The VariScreen Algorithm for LEV was not perfect with an indeterminate zone but it offered clinically relevant prediction with 88% PPV; moreover, the patients with false positive of VariScreen for LEV had small EV (table 11).

TABLE 11
Distribution of small EV as a function of VariScreen algorithm;
patient number in derivation population (211 patients).

LEV

| UGIE | Absence | Indeterminate | Presence |
|---|---|---|---|
| Esophageal varices: | | | |
| Absence | 98 | 17 | 0 |
| Small | 36 | 26 | 2 |
| Large | 6 | 16 | 14 |

In addition, 96-99% NPV and 100% specificity were obtained in substantial patient proportions. The two latter figures were obtained with CirrhoMeter^{VIRUS2G} which is the only available test specifically designed for cirrhosis diagnosis. It includes, hyaluronate which was the most accurate blood marker for LEV in the present study and elsewhere, and platelets and prothrombin index that are known markers for LEV.

### Conclusion

The non-invasive diagnosis of LEV exhaustively applied to CLD is superior to the conventional attitude based on liver biopsy or clinics followed by endoscopy in all patients with cirrhosis in terms of saved endoscopy. Likewise, the use of specific cut off for LEV of a blood test spared endoscopy compared to the strategy using cut-off of this blood test for cirrhosis followed by endoscopy. This sparing effect can be significantly improved by ECE. Therefore, in the era of non-invasive testing, tests should be primarily focused on cirrhosis complications screening, like LEV, rather on cirrhosis diagnosis itself.

### Example 2

### Evaluation and Improvement of Baveno 6 Recommendation for Non-Invasive Diagnosis of Esophageal Varices

### Introduction

Screening for esophageal varices (EV) is recommended in cirrhosis. The Baveno6 recommendations allow ruling out EV if platelets >150 G/l and Fibroscan <20 kPa. However, primary prevention focuses on large EV (LEV) and it is unknown in which etiology this rule applies. Therefore, we evaluated this rule and tried to improve it with the aim of 100% predictive values (NPV, PPV).

### Methods

287 patients with cirrhosis of various causes were prospectively included. Diagnostic tools were UGI endoscopy, 16 blood fibrosis tests, and Fibroscan. Patient characteristics were: men: 72%, age: 55+11 years, causes: alcohol: 64%, virus: 26%, NAFLD: 6%, others: 4%; EV: none: 56%, small: 27%, large: 17%.

### Results

Evaluation: NPV of Baveno6 rule was: EV: 87.1%, LEV: 100%. The spared endoscopy rate was only 16.4%. This rate was 38% with the best performing blood test (CirrhoMeter (CM), p<0.001 vs Baveno 6) for a missed LEV rate not significantly different (0%, 7%, respectively, p=0.157).

Improvement: A modified Baveno 6 rule (different cut-offs for platelets and Fibroscan) for EV had NPV100% in 18.2% of patients and even a PPV100% in 10.3% of patients. For LEV, there was a NPV100% in 37.0% of patients but no PPV100%. Finally, CM and Fibroscan combination had, respectively EV and LEV, NPV100% in 17.6% and 24.2% of patients and PPV100% in 6.7% and 3.0% of patients.

Discussion: The Baveno 6 rule has only a fair NPV for EV whereas it is very specific and poorly sensitive for LEV. New cut-offs provide NPV100% for LEV in more patients (37% vs 16%, p<0.001). By replacing platelets by a blood test, one can also get a 100% PPV. Thus, the best strategy is to use the modified Baveno 6 rule to rule out LEV and replace platelets by CM to rule in LEV. This algorithm has 100% accuracy with 0% missed LEV and 53.2% spared endoscopy. In practice, one measures platelets and stiffness in all patients; if the NPV100% cut-off is not reached, CM is performed; if the CM PPV100% cut-off is not reached, endoscopy is performed. The non-invasive strategy can be made in 1 or 2 steps knowing that the 2 non-invasive tests are already part of EASL and AASLD 2015 recommendations for fibrosis staging.

### Conclusion

The Baveno 6 rule can be notably improved. With 2 simple non-invasive tests and without additional cost, it is possible not only to rule out but also to rule in LEV, which is original, with any missed LEV and half of endoscopies spared. These results have to be validated in another population.

### Example 3: Large Esophageal Varice Screening with a Cirrhosis Blood Test Alone or Combined with Capsule Endoscopy in Chronic Liver Diseases

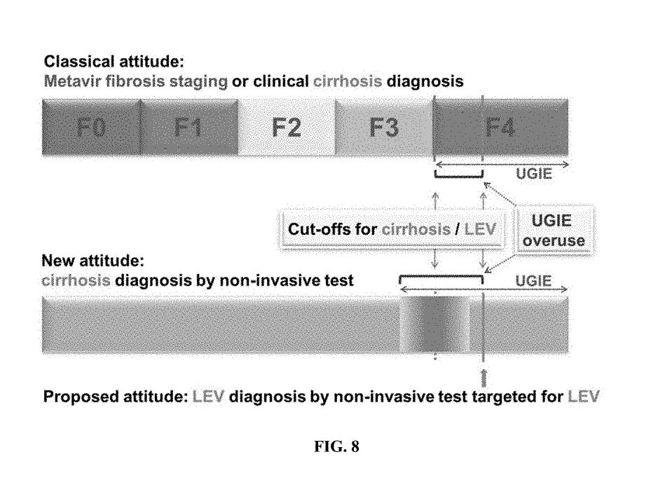

The conventional management of patients with suspected liver cirrhosis suffers from several limitations. First, several surveys [5] have reported that LEV screening policies based on UGIE are not well applied, which is probably attributable to the aforementioned constraints encountered by physicians in real-life clinical practice. Second, classical liver biopsy cannot be easily repeated; this may allow asymptomatic cirrhosis to go undetected, only to be revealed later by the development of complications. Therefore, improving the non-invasive diagnosis of cirrhosis is indeed an attractive option, but this too has limits and implications. First, an earlier diagnosis introduces a risk of UGIE overuse (FIG. 8). Indeed, recent guidelines stated that “HCV patients who were diagnosed with cirrhosis based on non-invasive diagnosis should undergo screening for PHT” [6]. Second, the construction and the evaluation of the performance of non-invasive fibrosis tests are limited by the characteristics of liver biopsy, which is an imperfect gold standard [7]. For all these reasons, a non-invasive test for LEV should ideally circumvent the intermediate step of cirrhosis diagnosis. It was hypothesized that cut-offs of non-invasive tests should be directly targeted for LEV detection, an endpoint that should be applicable in CLD generally (FIG. 8).

The main objective of the present study was thus to develop a diagnostic strategy for LEV screening based on non-invasive and/or minimally-invasive tests. Toward this, ECE, liver elastography and fibrosis blood tests were tested, either alone or combined, in patients with cirrhosis. The secondary objective was to assess the exportability (i.e. generalizability) of the non-invasive LEV diagnostic strategy to the general CLD population, where non-invasive fibrosis test would be ultimately used.

### Patients and Methods

### Patient Populations

The derivation population was extracted from a prospective study comparing ECE and UGIE for the diagnosis of LEV (large esophageal varices) in patients with cirrhosis of various etiologies recruited from April 2010 to March 2013 [8]. The 287 patients in whom both ECE and UGIE were performed were included. Diagnostic algorithms were developed in this derivation population of patients with cirrhosis.

The validation population included 165 patients with CLD attributed to viral infection or alcohol use, with or without cirrhosis, who had all undergone UGIE [9, 10]. This was a prospective study where UGIE was indicated to evaluate PHT signs. Blood tests and liver biopsy were available for all of the patients and all fibrosis stages were represented. However, these patients did not undergo ECE. Thus, this population was used to validate only the non-invasive strategy.

### Diagnostic Tools

Endoscopic procedures are detailed elsewhere [8, 11]. In both populations, EV size was classified into three grades: small, medium or large [10]. The two last grades were grouped as LEV. Sixteen blood tests were calculated (details in supplemental material). Among them, CirrhoMeterV2G, called CirrhoMeter hereafter, offered the highest performance. Vibration-controlled transient elastography (VCTE) (Fibroscan, Echosens, Paris, France) was performed according to the manufacturer's recommendations [12] by operators blinded to the other results.

### Costs Analysis

In this kind of study, only direct costs can be calculated. The costs of tests were those of the French list of care costs: CirrhoMeter: €29, UGIE: €114 (applying the 38% rate of general anesthesia recorded in the pivotal study), ECE: €612, liver biopsy: €1050 (including day hospitalization). The direct costs were calculated in the validation population, which was the only population where both non-invasive fibrosis and LEV tests were evaluated in the setting of clinical care.

### Study Design

### Diagnostic Algorithms

Ten diagnostic strategies were evaluated (see Table 12): five comprised a single diagnostic test and five a combination of several tests. These combinations were either simultaneous or sequential. When we were developing strategies, the priority objective was a missed LEV rate ≤5%.

TABLE 12
Different diagnostic strategies for LEV developed in the derivation population (with common size: n =
158) according to high negative and positive predictive value zones for LEV. The indeterminate zone lies
between the two previous zones and corresponds to an endoscopy requirement. Figures in brackets are 95% CI.

| | Large esophageal varices | | | | | | | |
|---|---|---|---|---|---|---|---|---|
| Strategy | Ruled out | | Indeterminate | | Ruled in | | Missed | |
| Single test: | | | | | | | | |
| ECE | | | | | | | | |
| Patients (%) | 59.5 | (51.6-67/5) | 29.1^{a} | (21.5-36.1) | 11.4 | (6.9-16.8) | 10.0 | (0-22.2) |
| Predictive value (%) | 96.8 | (92.9-100) | — | | 83.3 ^{b} | (62.5-100) | — | |
| VCTE | | | | | | | | |
| Patients (%) | 47.5 | (40.0-55.7) | 52.5 | (44.4-60.1) | 0 ^{c} | (0-0) | 13.3 | (2.7-26.1) |
| Predictive value (%) | 94.7 | (88.6-98.7) | — | | — | | | |
| ((AST/ALT) + PI) score | | | | | | | | |
| Patients (%) | 55.1 | (46.7-62.3) | 44.3 | (36.7-51.9) | 0.6 | (0-2.0) | 16.7 | (3.7-30.8) |
| Predictive value (%) | 94.3 | (89.3-98.8) | — | | 100 | (100-100) | — | |
| ((AST/ALT) + hyaluronate) score | | | | | | | | |
| Patients (%) | 54.4 | (46.5-62.0) | 44.3 | (35.5-51.9) | 1.3 | (0-3.2) | 20.0 | (6.1-35.1) |
| Predictive value (%) | 93.0 | (87.5-97.9) | — | | 100 | (100-100) | — | |
| CirrhoMeter (unadjusted) | | | | | | | | |
| Patients (%) | 55.7 | (47.7-63.1) | 40.5 | (33.1-48.1) | 3.8 | (1.2-7.2) | 13.3 | (3.0-27.3) |
| Predictive value (%) | 95.5 | (90.4-99.0) | — | | 83.3 | (NA) | — | |
| Simultaneous combination: | | | | | | | | |
| (ECE + (AST/ALT)) score | | | | | | | | |
| Patients (%) | 78.5 | (72.1-84.8) | 11.4 | (6.3-16.5) | 10.1 | (5.7-15.1) | 26.7 | (11.5-44.1) |
| Predictive value (%) | 93.5 | (88.9-97.6) | — | | 93.8 | (76.9-100) | — | |
| (ECE + CirrhoMeter) score | | | | | | | | |
| Patients (%) | 60.8 | (53.1-68.4) | 32.9 | (25.3-40.4) | 6.3 | (3.0-11.0) | 13.3 | (2.7-27.6) |
| Predictive value (%) | 95.8 | (91.6-99.0) | — | | 100 | (100-100) | — | |
| Sequential combination: | | | | | | | | |
| VCTE/ECE | | | | | | | | |
| Patients (%) | 47.5 | (40.0-55.7) | 43.0 | (35.4-51.3) | 9.5 ^{d} | (5.1-14.2) | 13.3 | (2.7-26.1) |
| Predictive value (%) | 94.7 | (88.7-98.7) | — | | 93.3 | (76.9-100) | — | |
| VCTE/(ECE + (AST/ALT)) score | | | | | | | | |
| Patients (%) | 47.5 | (40.0-55.7) | 43.7 | (36.1-51.9) | 8.9 ^{e} | (4.7-13.5) | 13.3 | (2.7-26.1) |
| Predictive value (%) | 94.7 | (89.0-98.8) | — | | 92.9 | (76.9-100) | — | |
| VariScreen algorithm ^{f} | | | | | | | | |
| Patients (%) | 58.9 | (51.2-66.2) | 29.7 | (22.6-37.1) | 11.4 | (6.5-16.4) | 6.7 | (0.0-16.7) |

| | | | | | | |
|---|---|---|---|---|---|---|
| Predictive value (%) | 97.8 | (94.3-100) | — | 88.9 | (72.2-100) | — |

| | | | | |
|---|---|---|---|---|
| p ^{g} | <0.001 | <0.001 | <0.001 | 0.648 |

LEV: large esophageal varices,
ECE: esophageal capsule endoscopy,
VCTE: vibration controlled transient elastography (Fibroscan).
Best results are shown in bold and worst in italics per zone and test category
^{a}Patients diagnosed with small EV by ECE
^{b} It was not possible to reach the objective (≥90%) as this is a semi-quantitative variable
^{c} Maximum PPV was <40%
^{d} This figure is different from ECE alone because three patients with high LEV PPV with ECE had high LEV NPV with VCTE
^{e} This figure is different from (ECE + (AST/ALT)) score alone (simultaneous combination) because two patients with high LEV PPV with (ECE + (AST/ALT)) score had high LEV NPV with VCTE
^{f} CirrhoMeter + (CirrhoMeter + ECE) score
^{g} By paired Cochran test for patient proportions

### Strategy Selection

Clinically applicable sequential strategies were selected. A clinically applicable strategy was defined as one including obligatorily a low constraint test (e.g. blood test) to rule out LEV (usually in asymptomatic patients) and possibly a high constraint test (e.g. UGIE) to rule in LEV (usually in the most severe patient cases).

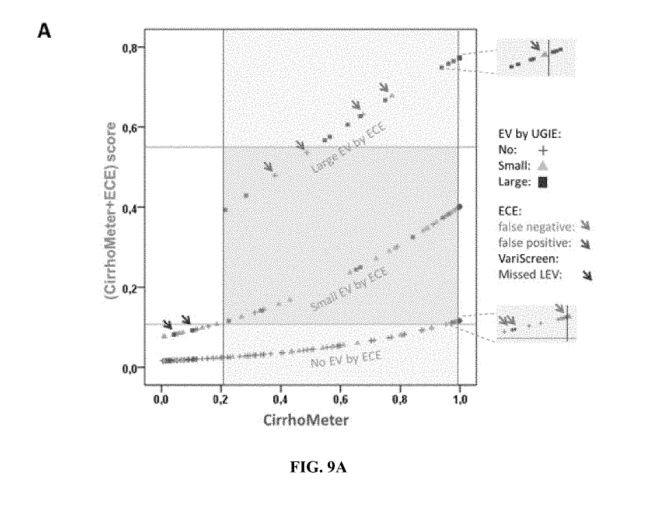

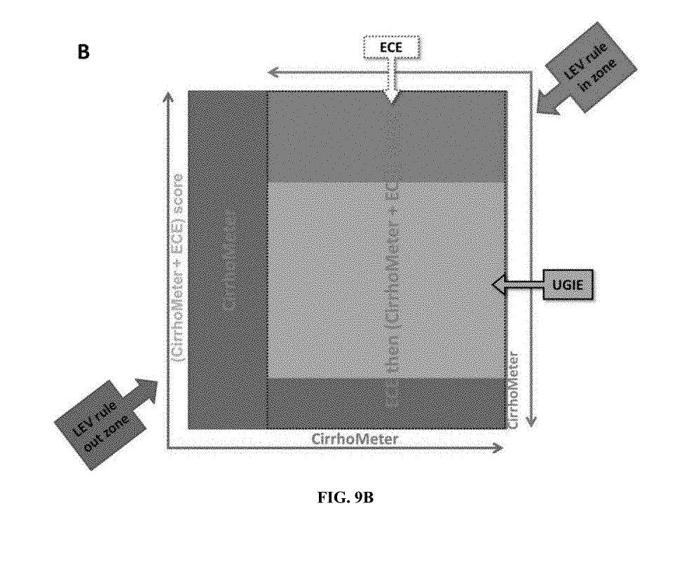

The most predictive of several clinically applicable strategies (details in Table 12) was to use CirrhoMeter first, mainly to rule out LEV (i.e. the test with the highest NPV criteria), then the combination of ECE and CirrhoMeter into a score to rule in LEV (positive predictive value (PPV)=93%). As there was a significant interaction (p<0.001) between these two tests, we analyzed their scatter plots (FIG. 9A), which showed that both tests had their own ruled in/out zones. Thus, UGIE was required only in the indeterminate zone common to the two tests (FIG. 9B). From these observations, we constructed a sequential diagnostic algorithm, called VariScreen hereafter, and presented in FIG. 10.

In practical terms, the VariScreen algorithm is as follows: CirrhoMeter is performed in all patients. Those with CirrhoMeter ≤0.21 are followed-up with yearly CirrhoMeter testing. Those with CirrhoMeter >0.9994 are offered primary prophylaxis. Patients between these two CirrhoMeter cut-offs are offered ECE. Then, the (ECE+CirrhoMeter) score is calculated by computer. Patients with (ECE+CirrhoMeter) scores <0.1114 are followed-up with yearly CirrhoMeter testing. Those with (ECE+CirrhoMeter) scores >0.55 are offered primary prophylaxis. Finally, patients between the two score cut-offs are offered UGIE, as they run a 23% probability of having LEV.

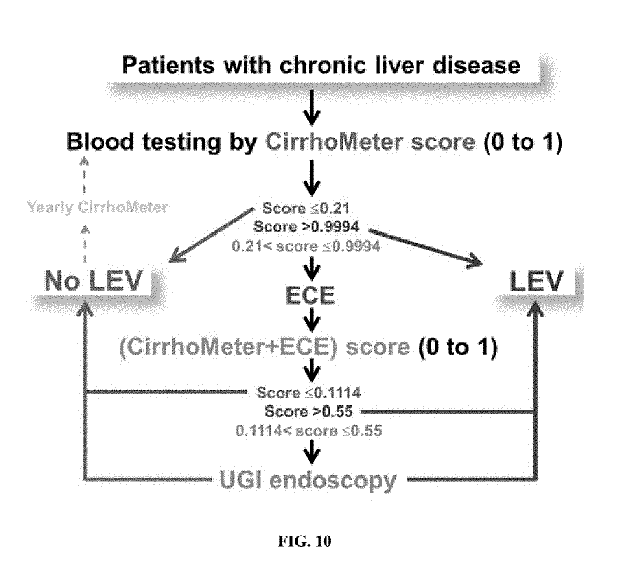

As VariScreen includes ECE, this is a partially minimally-invasive strategy. Therefore, an entirely non-invasive strategy was also developed. FIG. 11 shows that FibroMeter targeted to significant fibrosis was synergistic with CirrhoMeter to rule out and in LEV. This association was called the CirrhoMeter+FibroMeter algorithm. Cut-offs were, respectively to rule out or in LEV, FibroMeter: 0.78/0.9993, CirrhoMeter: 0.21/0.998.

### Statistics

### Clinical Descriptors

Missed LEV—This was the proportion of patients with undetected LEV in the patient subgroup with LEV.

UGIE requirement—This was the proportion of patients in the indeterminate zone of the non-invasive tests, i.e. between their negative predictive value (NPV) and PPV cut-offs for LEV.

Spared UGIE—Patients with cirrhosis were used as the reference group to calculate the rate of patients that the algorithm would spare from UGIE, as this latter is classically performed in these patients. This comprised the entire derivation population and patients with Metavir F4 stage by liver biopsy or with cirrhosis diagnosed by CirrhoMeter in the validation population. The spared UGIE rate corresponds to the difference between the cirrhosis group and the LEV target group where UGIE was indicated by non-invasive tests. Thus, non-invasive tests might be used with cut-offs for cirrhosis diagnosis or LEV diagnosis.

### Diagnostic Test Segmentation

For LEV diagnosis, we initially determined the two cut-offs of a test value to reach a NPV≥295% and a PPV≥290%. Consequently, these two cut-offs determined three diagnostic zones: LEV ruled out (≤NPV cut-off), indeterminate, and LEV ruled in (≥PPV cut-off). In the final diagnostic algorithm, the cut-offs of constitutive tests were adjusted to minimize the missed LEV rate (priority clinical objective) if necessary.

### Statistical Descriptors and Tests

Quantitative variables were expressed as mean±standard deviation. The discriminative ability of each test was expressed as the area under the receiver operating characteristic (AUROC) curve and compared by the Delong test. Data were reported according to STARD [13] and Liver FibroSTARD [14] statements, and analyzed on an intention-to-diagnose basis. Scores including independent LEV predictors were determined by binary logistic regression. The main statistical analyses were performed under the control of professional statisticians (SB, GH) using SPSS version 18.0 (IBM, Armonk, N.Y., USA) and SAS version 9.3 (SAS Institute Inc., Cary, N.C., USA).

### Results

### Population Characteristics

The characteristics of the populations are provided in Table 2 hereinabove. In the validation CLD population, LEV were only observed in patients with cirrhosis confirmed by liver biopsy and in those with probable cirrhosis (estimated Metavir classes F3/4 and F4) according to CirrhoMeter.

### Overall Test Accuracy

AUROCs for LEV are detailed in Table 4 hereinabove. Briefly, in the derivation population, the highest AUROCs (≥0.91) were obtained with two scores combining ECE and blood markers. The AUROCs of these scores were significantly higher than those of fibrosis tests (p<0.02), whereas the AUROCs between fibrosis tests were not significantly different (details not shown). In the validation population, the AUROC of Metavir F stages by liver biopsy (0.819) was significantly lower (p<0.01) than that of the most accurate blood fibrosis tests (e.g. 0.911 for CirrhoMeter).

### Strategy Development

CirrhoMeter was the best performing low constraint test, providing the largest ruled out zone and the highest measured NPV for LEV (Table 13).

ECE was the best of the five single test strategies (details in the supplemental material), providing a significantly lower proportion of indeterminate patients and the largest ruled in zone. However, its PPV for LEV was 80% (Table 13), falling short of the targeted 90% value. Among the five combination strategies, the (ECE+CirrhoMeter) score provided the highest measured PPV for LEV (Table 13).

TABLE 13
Comparison of LEV prediction between all four strategies based on CirrhoMeter
(CM) and/or esophageal capsule endoscopy (ECE) as a function of test zones.
Figures in brackets are 95% CI. Derivation population (211 patients).

| | Large esophageal varices | | | | |
|---|---|---|---|---|---|
| Strategy | Ruled out | | Indeterminate ^{a} | Ruled in | |
| 1. ECE | | | | | |
| Patients (%) | 58.8 | (52.6-65.7) | 29.4 (23.3-35.5) | 11.8 | (7.6-16.5) |
| Predictive value (%) | 97.6 | (94.9-100) | 21.0 (10.3-30.6) | 80.0 | (61.9-95.0) |
| 2. CM (unadjusted) | | | | | |
| Patients (%) | 53.1 | (46.7-60.1) | 44.1 (37.1-50.7) | 2.8 | (0.9-5.2) |
| Predictive value (%) | 94.6 | (89.9-98.3) | 26.9 (17.6-36.4) | 83.3 | (NA) |
| 3. (ECE + CM) score | | | | | |
| Patients (%) | 59.7 | (53.1-66.0) | 33.6 (27.4-40.4) | 6.6 | (3.7-10.0) |
| Predictive value (%) | 96.8 | (93.4-99.2) | 26.8 (16.7-37.8) | 92.9 | (76.9-100) |
| 4. VariScreen algorithm ^{b} | | | | | |
| Patients (%) | 58.3 | (51.5-64.8) | 30.8 (24.5-37.2) | 10.9 | (6.8-15.1) |
| Predictive value (%) | 98.4 | (95.7-100) | 23.1 (13.0-33.9) | 82.6 | (66.7-96.4) |
| Comparison (p) ^{c} | | | | | |

| | | | |
|---|---|---|---|
| All 4 strategies | 0.082 | <0.001 | <0.001 |
| ECE vs CM | 0.188 | 0.001 | <0.001 |
| ECE vs (ECE + CM) score | 0.856 | 0.211 | 0.001 |
| ECE vs VariScreen | 1 | 0.743 | 0.687 |
| CM vs (ECE + CM) score | 0.038 | 0.003 | 0.039 |
| CM vs VariScreen | 0.099 | <0.001 | <0.001 |
| (ECE + CM) score vs VariScreen | 0.250 | 0.146 | 0.004 |

NA: not available
^{a} With positive predictive value for LEV
^{b} CirrhoMeter + (CirrhoMeter + ECE) score
^{c} Patients proportions: between the four strategies by paired Cochran test and for pairwise comparison by paired McNemar test

### Algorithm Evaluation

### Derivation Population

### CirrhoMeter+ FibroMeter Algorithm

Table 14 shows that CirrhoMeter targeted for cirrhosis had to be used with its three classes including F4 to miss <5% LEV. Spared UGIE was then 15.6%. However, CirrhoMeter and the CirrhoMeter+FibroMeter algorithm targeted for LEV significantly increased (p<0.001) spared UGIE to 36.0 and 43.1%, respectively, the latter figure being significantly higher than the former (p<0.001). In other words, targeting CirrhoMeter to LEV reduced UGIE by 14.4% (p<0.001) compared to targeting it for cirrhosis.

TABLE 14
Rates (%) of diagnostic indices for cirrhosis, spared endoscopy (UGIE) and missed large esophageal
varices (LEV) using different cut-offs of blood tests targeted for cirrhosis or LEV. The reference
for calculation of spared UGIE and missed LEV is either cirrhosis diagnosis by clinics (derivation
population) or liver biopsy (validation population), or CirrhoMeter targeted for LEV.

| | Cirrhosis | | Spared UGIE | | Missed LEV | |
|---|---|---|---|---|---|---|
| Reference for UGIE | PPV | Se | Cirrhosis | CM ^{a} | Cirrhosis | CM ^{a} |

Derivation population ^{b}:
CirrhoMeter targeted for cirrhosis ^{c}:

| | | | | | | | | | | |
|---|---|---|---|---|---|---|---|---|---|---|
| F3 ± 1 + F3/4 + F4 | — ^{d} | 84.4 | 15.6 | (p < 0.001) | −14.4 | (p < 0.001) | 2.8 | (p = 0.317) | 2.8 | (p = 1) |
| F3/4 + F4 | — ^{d} | 63.5 | 36.5 | (p < 0.001) | −0.5 | (p = 1) | 11.1 | (p = 0.046) | 5.5 | (p = 0.500) |
| F4 | — ^{d} | 38.9 | 61.1 | (p < 0.001) | 25.1 | (p < 0.001) | 33.3 | (p < 0.001) | 27.7 | (p = 0.002) |

Test targeted for LEV:

| CirrhoMeter | — ^{d} | 64.0^{e} | 36.0 | (p < 0.001) | — | | 5.6 | (p = 0.157) | — | |
|---|---|---|---|---|---|---|---|---|---|---|
| CirrhoMeter + FibroMeter | — ^{d} | 56.9^{e} | 43.1 | (p < 0.001) | 7.1 | (p < 0.001) | 5.6 | (p = 0.157) | 0 | (p = 1) |

Validation population:
CirrhoMeter targeted for cirrhosis^{c}:

| | | | | | | | | | | |
|---|---|---|---|---|---|---|---|---|---|---|
| F3 ± 1 + F3/4 + F4 | 72.4 | 93.4 | −28.9 | (p < 0.001) | −19.4 | (p < 0.001) | 0 | (p = 1) | 0 | (p = 1) |
| F3/4 + F4 | 84.0 | 82.9 | 1.3 | (p = 1) | 5.3 | (p = 0.125) | 0 | (p = 1) | 0 | (p = 1) |
| F4 | 92.6 | 65.8 | 28.9 | (p < 0.001) | 46.3 | (p < 0.001) | 9.7 | (p = 0.083) | 9.7 | (p = 0.083) |

Test targeted for LEV:

| CirrhoMeter | 81.0 | 84.2^{e} | −3.9 | (p = 0.701) | — | | 0 | (p = 1) | — | |
|---|---|---|---|---|---|---|---|---|---|---|
| CirrhoMeter + FibroMeter | 81.8 | 82.9^{e} | −1.3 | (p = 1) | 2.5 | (p = 0.500) | 0 | (p = 1) | 0 | (p = 1) |

PPV: positive predictive value,
Se: sensitivity,
p: comparison vs reference by paired McNemar test
^{a} CirrhoMeter targeted for LEV
^{b} The rates were calculated in the derivation population with maximum size (n = 211)
^{c} CirrhoMeter fibrosis classification includes 6 classes, 3 of which include F4: F3 ± 1 + F3/4 + F4
^{d} PPV is artificially at 100% due to cirrhosis population selection
^{e}Corresponds to the indeterminate zone for LEV; there was no PPV zone for LEV in the validation population

### VariScreen Algorithm

Comparisons for the VariScreen algorithm and its constitutive tests are presented in Table 15. Briefly, the missed LEV rates were not significantly different between the tests. ECE accuracy was significantly lower than in other tests. Considering spared UGIE rates, all tests were significantly different except VariScreen and ECE. Thus, there was a progressive increase in spared UGIE, CirrhoMeter targeted for cirrhosis: 15.6%, CirrhoMeter targeted for LEV: 36.0% (p<0.001 vs previous), CirrhoMeter+FibroMeter algorithm: 43.1% (p<0.001 vs previous), ECE and VariScreen: around 70% (p<0.001 vs previous).

TABLE 15
Comparison of diagnostic performance (%) of noteworthy diagnostic tests
in their categories in the derivation population (211 patients).

| | Accuracy | UGIE | LEV |
|---|---|---|---|
| | LEV ^{a} | spared | missed |
| CirrhoMeter cirrhosis ^{b} | 99.5 | 15.6 | 2.8 |
| CirrhoMeter LEV ^{c} | 98.6 | 36.0 | 5.6 |
| CirrhoMeter + FibroMeter | 96.7 | 43.1 | 5.6 |
| ECE | 90.0 | 70.6 | 8.3 |
| VariScreen | 97.2 | 69.2 | 5.6 |
| Comparison (p) ^{d} | | | |
| All | <0.001 | <0.001 | 0.789 |
| CM F4 vs. CM LEV | 0.500 | <0.001 | 1 |
| CM F4 vs. CM + FM | 0.031 | <0.001 | 1 |
| CM F4 vs. ECE | <0.001 | <0.001 | 0.625 |
| CM F4 vs. VS | 0.063 | <0.001 | 1 |
| CM LEV vs. CM + FM | 0.125 | <0.001 | 1 |
| CM LEV vs. ECE | <0.001 | <0.001 | 1 |
| CM LEV vs. VS | 0.250 | <0.001 | 1 |
| CM + FM vs ECE | 0.004 | <0.001 | 1 |
| CM + FM vs VS | 1 | <0.001 | 1 |
| ECE vs VS | <0.001 | 0.743 | 1 |

LEV: large esophageal varices,
UGIE: upper gastrointestinal endoscopy,
ECE: esophageal capsule endoscopy,
CM: CirrhoMeter,
FM: FibroMeter,
F4: cirrhosis,
VS: VariScreen
^{a} Correctly classified patients for LEV
^{b} CirrhoMeter with cut-off targeted for cirrhosis
^{c} CirrhoMeter with adjusted cut-off targeted for LEV
^{d} Paired Cochran test for global comparison and paired McNemar test for pair comparisons

VariScreen accuracy was not dependent on Child-Pugh classes: A: 97.7%, B: 94.4%%, C: 97.3%, p=0.587).

The distribution of small EV and gastric varices as a function of the VariScreen algorithm is depicted in Table 16.

TABLE 16
Distribution of esophageal varices and gastric varices by UGIE
as a function of VariScreen ruled in/out and indeterminate zones;
patient number in the derivation population (211 patients).

Large esophageal varices

| UGIE | Ruled out | Indeterminate | Ruled in |
|---|---|---|---|
| Esophageal varices: | | | |
| Absent | 94 | 20 | 1 |
| Small | 27 | 30 | 3 |
| Large | 2 | 15 | 19 |
| Gastric varices: | | | |
| Absent | 120 | 64 | 18 |
| Present | 3 | 1 | 5 |

Misclassified patients for LEV or gastric varices by VariScreen are shown in bold

Misclassified patients—Twenty-one patients (10.0%) were misclassified for LEV by ECE, including 5 false positives of which 2 were rescued by VariScreen (FIG. 9) and 16 false negatives of which 14 were rescued by VariScreen. Thus, VariScreen rescued 16 patients (76.2%) from ECE misclassification. However, VariScreen misclassified one of the LEV cases correctly classified by ECE. Thus, the net result was 16-1=15, corresponding to the 7.1% gain in accuracy with VariScreen compared to ECE. Among the 16 LEV false negatives by ECE, 3 were particularly discrepant as no EV were seen on ECE (FIG. 9). These 3 patients had significantly worse liver statuses (details not shown) compared to other patients with no EV by ECE, suggesting true false negatives and justifying the NPV cut-off of the (CirrhoMeter+ECE) score used in VariScreen (FIG. 9).

Six patients were misclassified for LEV by VariScreen, specifically 4 false positives and 2 false negatives. The 2 false negative patients had blood markers significantly different (reflecting a better liver status) from other patients with LEV, e.g. median serum albumin levels (g/l) in patients with LEV: ruled out zone (i.e. the 2 missed LEV): 41.5; indeterminate zone: 31.0; ruled in zone: 27.0; p=0.040 by Kruskal-Wallis test. Among the 4 false positive cases, 3 had small EV, explaining the VariScreen PPV for EV of 97%.

Comparison with recommendation—The Baveno VI rule had high NPV: 86.2% for EV and 100% for LEV, but the spared UGIE rate was only 18.4% vs 70.3% (p<0.001) with VariScreen or 38.0% with CirrhoMeter targeted for LEV (p<0.001) while missed LEV rates were not significantly different (details in Table 17).

TABLE 17
Comparison of rates (%) of spared endoscopy (UGIE) and
missed large esophageal varices (LEV) between all strategies
based on CirrhoMeter and the Baveno VI rule in the derivation
population with maximum size (n = 158).

| Strategy ^{a} | Spared UGIE | Missed LEV |
|---|---|---|
| CirrhoMeter unadjusted ^{b} | 59.5 | 13.3 |
| CirrhoMeter adjusted ^{b} | 38.0 | 6.7 |
| CirrhoMeter + FibroMeter | 43.7 | 6.7 |
| Baveno VI rule ^{c} | 18.4 | 0 |
| VariScreen algorithm ^{d} | 70.3 | 6.7 |
| p ^{e} | — | — |
| All | <0.001 | 0.040 |
| Baveno VI vs: | — | — |
| CirrhoMeter unadjusted | <0.001 | 0.046 |
| CirrhoMeter adjusted | <0.001 | 0.157 |
| CirrhoMeter + FibroMeter | <0.001 | 0.157 |
| VariScreen | <0.001 | 0.157 |

Best results are shown in bold
^{a} Cut-offs of CirrhoMeter and scores were defined a posteriori for LEV in the derivation population
^{b} Cut-offs of CirrhoMeter used alone, either unadjusted or adjusted (as used in VariScreen)
^{c} Spared UGIE when VCTE < 20 kPa and platelets > 150 G/l
^{d} CirrhoMeter + (CirrhoMeter + ECE) score
^{e} By paired Cochran test between the four proportions. Pairwise comparisons by paired Wilcoxon test

### Validation Population

The CirrhoMeter and the CirrhoMeter+FibroMeter algorithm targeted for LEV did not significantly reduce UGIE compared to cirrhosis diagnosis by liver biopsy but they did compared to CirrhoMeter targeted for cirrhosis, e.g. 21.9% (p<0.001) for CirrhoMeter+FibroMeter algorithm (Table 14). Importantly, the missed LEV rate was 0%.

### Costs Analysis

The strategies with minimal missed LEV rates (0.3%) are analyzed in terms of costs. The most expensive strategy was the classical strategy based on initial cirrhosis diagnosis by liver biopsy (Table 18). The least expensive strategy was that based on CirrhoMeter or CirrhoMeter+ FibroMeter targeted for LEV. The addition of ECE multiplied the cost of the latter by 4.2 (or 3.1 vs CirrhoMeter targeted for cirrhosis) but VariScreen was 3.5 times less expensive than the classical strategy based on fibrosis staging by liver biopsy.

TABLE 18
Cost-efficacy analysis in the validation population.

| | Spared UGIE ^{a} | Missed LEV | Mean cost |
|---|---|---|---|
| Strategy | (%) | (%) | (€/patient) |
| CirrhoMeter for cirrhosis: | | | |
| F3 ± 1, F3/4 and F4 | −28.9 | 0 | 102 |
| F3/4 and F4 | 1.3 | 0 | 85 |
| F4 | 28.9 | 9.7 | 70 |
| Test targeted for LEV: | | | |
| CirrhoMeter | −3.9 (19.4 ^{b}) | 0 | 88 |
| CirrhoMeter + FibroMeter ^{c} | −1.3 (21.9 ^{b}) | 0 | 87 |
| VariScreen algorithm | 53.9 ^{d} | 0 ^{e} | 316 |
| Liver biopsy | 0 | 0 | 1106 |

UGIE: upper gastrointestinal endoscopy,
LEV: large esophageal varices
^{a} The reference population is cirrhosis unless otherwise stated
^{b} The reference population is cirrhosis diagnosed by CirrhoMeter
^{c} No additional cost for FibroMeter
^{d} Calculation estimated by applying the rate of spared UGIE by (CirrhoMeter + ECE) score in the indeterminate CirrhoMeter zone of the derivation population (48.1%) assuming a robust ECE performance and using the cut-offs of the VariScreen algorithm
^{e} Calculation assuming that missed LEV are attributable to CirrhoMeter

### REFERENCES

[1] de Franchis R. Revising consensus in portal hypertension: report of the Baveno V consensus workshop on methodology of diagnosis and therapy in portal hypertension. J Hepatol 2010; 53:762-768.
[2] de Franchis R, Dell'Era A. Invasive and noninvasive methods to diagnose portal hypertension and esophageal varices. Clinics in liver disease 2014; 18:293-302.
[3] Colli A, Gana J C, Turner D, Yap J, Adams-Webber T, Ling S C, et al. Capsule endoscopy for the diagnosis of oesophageal varices in people with chronic liver disease or portal vein thrombosis. The Cochrane database of systematic reviews 2014; 10:CD008760.
[4] de Franchis R, Baveno VIF. Expanding consensus in portal hypertension: Report of the Baveno VI Consensus Workshop: Stratifying risk and individualizing care for portal hypertension. J Hepatol 2015; 63:743-752.
[5] Buchanan P M, Kramer J R, El-Serag H B, Asch S M, Assioun Y, Bacon B R, et al. The quality of care provided to patients with varices in the department of Veterans Affairs. Am J Gastroenterol 2014; 109:934-940.
[6] European Association for the Study of the Liver. Electronic address eee, Asociacion Latinoamericana para el Estudio del Higado. EASL-ALEH Clinical Practice Guidelines: Non-invasive tests for evaluation of liver disease severity and prognosis. J Hepatol 2015; 63:237-264.
[7] Bedossa P, Carrat F. Liver biopsy: the best, not the gold standard. J Hepatol 2009; 50:1-3.
[8] Sacher-Huvelin S, Cales P, Bureau C, Valla D, Vinel J P, Duburque C, et al. Screening of esophageal varices by esophageal capsule endoscopy: results of a French multicenter prospective study. Endoscopy 2015; 47:486-492.
[9] Oberti F, Valsesia E, Pilette C, Rousselet M C, Bedossa P, Aube C, et al. Noninvasive diagnosis of hepatic fibrosis or cirrhosis. Gastroenterology 1997; 113:1609-1616.
[10] Pilette C, Oberti F, Aube C, Rousselet M C, Bedossa P, Gallois Y, et al. Non-invasive diagnosis of esophageal varices in chronic liver diseases. J Hepatol 1999; 31:867-873.
[11] Oberti F, Burtin P, Maiga M, Valsesia E, Pilette C, Cales P. Gastroesophageal endoscopic signs of cirrhosis: independent diagnostic accuracy, interassociation, and relationship to etiology and hepatic dysfunction. Gastrointest Endose 1998; 48:148-157.
[12] Castera L, Forns X, Alberti A. Non-invasive evaluation of liver fibrosis using transient elastography. J Hepatol 2008; 48:835-847.
[13] Bossuyt P M, Reitsma J B, Bruns D E, Gatsonis C A, Glasziou P P, Irwig L M, et al. The STARD statement for reporting studies of diagnostic acuracy: explanation and elaboration. Clin Chem 2003; 49:7-18.
[14] Boursier J, de Ledinghen V, Poynard T, Guechot J, Carrat F, Leroy V, et al. An extension of STARD statements for reporting diagnostic accuracy studies on liver fibrosis tests: the Liver-FibroSTARD standards. J Hepatol 2015; 62:807-815.
[15] Stefanescu H, Radu C, Procopet B, Lupsor-Platon M, Habic A, Tantau M, et al. Non-invasive menage a trois for the prediction of high-risk varices: stepwise algorithm using lok score, liver and spleen stiffness. Liver Int 2015; 35:317-325.
[16] Berzigotti A, Seijo S, Arena U, Abraldes J G, Vizzutti F, Garcia-Pagan J C, et al. Elastography, spleen size, and platelet count identify portal hypertension in patients with compensated cirrhosis. Gastroenterology 2013; 144:102-111 e101.
[17] Cales P, Zabotto B, Meskens C, Caucanas J P, Vinel J P, Desmorat H, et al. Gastroesophageal endoscopic features in cirrhosis. Observer variability, interassociations, and relationship to hepatic dysfunction. Gastroenterology 1990; 98:156-162.
[18] Singh S, Eaton J E, Murad M H, Tanaka H, Iijima H, Talwalkar J A. Accuracy of spleen stiffness measurement in detection of esophageal varices in patients with chronic liver disease: systematic review and meta-analysis. Clin Gastroenterol Hepatol 2014; 12:935-945 e934.
[19] Bureau C, Metivier S, Peron J M, Selves J, Robic M A, Gourraud P A, et al. Transient elastography accurately predicts presence of significant portal hypertension in patients with chronic liver disease. Aliment Pharmacol Ther 2008; 27:1261-1268.
[20] Castera L, Le Bail B, Roudot-Thoraval F, Bernard P H, Foucher J, Merrouche W, et al. Early detection in routine clinical practice of cirrhosis and oesophageal varices in chronic hepatitis C: comparison of transient elastography (FibroScan) with standard laboratory tests and non-invasive scores. J Hepatol 2009; 50:59-68.
[21] Boursier J, Bacq Y, Halfon P, Leroy V, de Ledinghen V, de Muret A, et al. Improved diagnostic accuracy of blood tests for severe fibrosis and cirrhosis in chronic hepatitis C. Eur J Gastroenterol Hepatol 2009; 21:28-38.
[22] Degos F, Perez P, Roche B, Mahmoudi A, Asselineau J, Voitot H, et al. Diagnostic accuracy of FibroScan and comparison to liver fibrosis biomarkers in chronic viral hepatitis: a multicenter prospective study (the FIBROSTIC study). J Hepatol 2010; 53:1013-1021.
[23] Zarski J P, Sturm N, Guechot J, Paris A, Zafrani E S, Asselah T, et al. Comparison of nine blood tests and transient elastography for liver fibrosis in chronic hepatitis C: The ANRS HCEP-23 study. J Hepatol 2012; 56:55-62.
[24] Boursier J, Ducancelle A, Leroy V, Vergniol J, Sturm N, Lebail B, et al. Comparison of 16 blood and/or elastometric fibrosis tests in 5 causes of chronic liver diseases: too much? Towards a simplification. Hepatology 2015; 62:912A.
[25] Cales P, Boursier J, Oberti F, Bardou D, Zarski J P, de Ledinghen V. Cirrhosis Diagnosis and Liver Fibrosis Staging: Transient Elastometry Versus Cirrhosis Blood Test. J Clin Gastroenterol 2015; 49:512-519.
[26] Boursier J, Brochard C, Bertrais S, Michalak S, Gallois Y, Fouchard-Hubert I, et al. Combination of blood tests for significant fibrosis and cirrhosis improves the assessment of liver-prognosis in chronic hepatitis C. Alimentary pharmacology & therapeutics 2014; 40:178-188.
[27] Cales P, Zarski J P, Marc Chapplain J, Bertrais S, Sturm N, Michelet C, et al. Fibrosis progression under maintenance interferon in hepatitis C is better detected by blood test than liver morphometry. J Viral Hepat 2012; 19:e143-153.
[28] Fontana R J, Sanyal A J, Ghany M G, Lee W M, Reid A E, Naishadham D, et al. Factors that determine the development and progression of gastroesophageal varices in patients with chronic hepatitis C. Gastroenterology 2010; 138:2321-2331, 2331 e2321-2322.
[29] Vanbiervliet G, Barjoan-Marine E, Anty R, Piche T, Hastier P, Rakotoarisoa C, et al. Serum fibrosis markers can detect large oesophageal varices with a high accuracy. Eur J Gastroenterol Hepatol 2005; 17:333-338.
[30] Sanyal A J, Fontana R J, Di Bisceglie A M, Everhart J E, Doherty M C, Everson G T, et al. The prevalence and risk factors associated with esophageal varices in subjects with hepatitis C and advanced fibrosis. Gastrointest Endosc 2006; 64:855-864.
[31] Cales P, Boursier J, Bertrais S, Oberti F, Gallois Y, Fouchard-Hubert I, et al. Optimization and robustness of blood tests for liver fibrosis and cirrhosis. Clin Biochem 2010; 43:1315-1322.
[32] Cales P, Boursier J, Oberti F, Hubert I, Gallois Y, Rousselet M C, et al. FibroMeters: a family of blood tests for liver fibrosis. Gastroenterologie clinique et biologique 2008; 32:40-51.

### Example 4: Algorithms for Non-Invasive Diagnosis of Large Esophageal Varices (LEV)

### Objective

The objective is to obtain a non-invasive diagnosis of large esophageal varices (LEV) with the following rules for statistical algorithms:
Non-invasive tests used with cut-offs allowing 100% predictive values (both negative predictive value and positive predictive value) for LEV (with some rare exceptions in a few algorithms),
Indication of UGI endoscopy for patients sorted in the grey intermediate zone located between the two cut-offs of negative and positive predictive values.

### Fibrosis Tests

### Blood Tests:

CirrhoMeter^{V2G} called CirrhoMeter (CM) thereafter and expressed as a score from 0 to 1.

FibroMeter^{V2G} called FibroMeter (FM) thereafter and expressed as a score from 0 to 1. Platelet (Pl) count expressed in G/l.

### Liver Elastography:

Fibroscan (FS) called vibration controlled transient elastography (VCTE) thereafter and expressed in kPa

### Population

Cirrhotic patients from the VO-VCO studies (Sacher-Huvelin Endoscopy 2015) (see description hereinabove in Examples 1 and 3):
221 patients with blood tests available,
165 patients with both VCTE and blood tests available.

### Simple Algorithms

They are based on single negative predictive value (NPV) zone and positive predictive value (PPV) zone according to classical statistical rules.

### Algorithm CMFM#1

This FibroMeter+CirrhoMeter algorithm for large esophageal varices is described in Example 3 (see FIG. 11).

The LEV rule out (NPV) zone is defined by the following cut-offs: CirrhoMeter <0.21 or FibroMeter <0.78.

The LEV rule in (PPV) zone is defined by the following cut-offs: CirrhoMeter >0.998 and FibroMeter >0.9993.

### Algorithm CMFM#1b

### Principles:

With this second FibroMeter+CirrhoMeter algorithm with different cut-off values, there is no missed LEV and less false positives (only one) compared to CMFM#1.

The LEV rule out zone is defined by the following cut-offs: CirrhoMeter <0.042 or FibroMeter <0.51

The LEV rule in zone is defined by the following cut-offs: CirrhoMeter >0.99945

### Algorithm PlFS#1

This is a Platelets+VCTE (also known as Fibroscan™) algorithm for large esophageal varices.

The LEV rule out zone is defined by the following cut-offs: platelets >110 G/l and VCTE<26.5 kPa.

The LEV rule in zone is defined by the following cut-offs: platelets <45 G/l and VCTE>32 kPa.

NB: PPV is 100% in 0 patients, i.e. no PPV zone.

### Algorithm PlFS#1b

This is another Platelets+VCTE (also known as Fibroscan™) algorithm for large esophageal varices.

### Principles:

### Baseline Algorithm:

PlFS#1 for LEV out zone

New rule for LEV rule in zone: presence with a minimum of false positives (only one in fact).

The LEV rule out zone is defined by the following cut-offs: platelets >110 G/l and VCTE<26.5 kPa.

The LEV rule in zone is defined by the following cut-offs: platelets <65 G/l and VCTE>32 kPa.

### NB:

PPV is 83.3% among 6 patients in a sample size of 165 patients
PPV is 85.7% among 7 patients in a sample size of 221 patients

### Algorithm CMFS#1

This CirrhoMeter+Fibrocan™ (also known as VCTE) algorithm is described hereinabove in Example 2.

The LEV rule out zone is defined by the following cut-offs: CirrhoMeter <0.6 and VCTE<14 kPa.

The LEV rule in zone is defined by the following cut-offs: CirrhoMeter >0.99891 and VCTE>55 kPa.

### Multiple Algorithms

They are based on multiple negative predictive value (NPV) zones and positive predictive value (PPV) zones according to new statistical rules as described in Example 5 below.

### Algorithm PlCMFS#1

This is a Platelets+CirrhoMeter+VCTE (also known as Fibroscan™) algorithm for large esophageal varices.

### Principles:

### Baseline Algorithm:

PlFS#1 for LEV out zone
CMFS#1 for LEV in zone

### Additional Zone for LEV Out Zone:

New CMFS rule

### LEV Rule Out Zone:

platelets >110 G/l and VCTE<26.5 kPa.
CirrhoMeter <0.334 and VCTE<35 kPa.

LEV rule in zone: CirrhoMeter >0.99891 and VCTE>55 kPa.

### Algorithm PlFMCMFS#I

This is a Platelets+FibroMeter+CirrhoMeter+VCTE (also known as Fibroscan™) algorithm for large esophageal varices.

### Principles:

Baseline algorithm: PlCMFS#1

### Additional Zone for LEV Out Zone:

CirrhoMeter <0.004 or VCTE<9.1 kPa
FibroMeter <0.05
FibroMeter <0.895 and VCTE<33 kPa

### Additional Zone for LEV in Zone:

FibroMeter >0.9994 and VCTE>60.

### Summary of Diagnostic Algorithms for LEV

TABLE 19
Algorithms carried out in a population where only blood
tests are available with a sample size of 221 patients.

| | Diagnostic | Spared UGIE | Missed LEV | Sample |
|---|---|---|---|---|
| Algorithm | accuracy (%) | (%) | (%) | size |
| CM | 98.6 | 36.7 | 5.6 | 221 |
| CMFM#1 | 96.8 ^{a} | 43.9 | 5.6 | 221 |
| CMFM#1b | 99.5 ^{b} | 15.4 | 0 | 221 |
| p | 0.009 | <0.001 | 0.135 | |

^{a} five false positive
^{b} one false positive
p by paired cochran's Q test

TABLE 20
Algorithms carried out in a population where blood tests and
VCTE are available with a sample size of 165 patients.

| | Diagnostic | Spared UGIE | Missed LEV | Sample |
|---|---|---|---|---|
| Algorithm | accuracy (%) | (%) | (%) | size |
| CM | 98.2 | 38.2 | 6.5 | 165 |
| CMFM#1 | 98.2 | 44.2 | 6.5 | 165 |
| CMFM#1b | 99.4^{a} | 17.0 | 0 | 165 |
| CMFS#1 | 100 | 27.3 | 0 | 165 |
| PlFS#1 | 100 | 37.0 | 0 | 165 |
| PlFS#1b | 99.4^{a} | 39.4 | 0 | 165 |
| PlCMFS#1 | 100 | 47.3 | 0 | 165 |
| PlFMCMFS#1 | 100 | 53.9 | 0 | 165 |
| p | 0.041 | <0.001 | 0.051 | 165 |

^{a}one false positive
p by paired cochran's Q test

### Synthesis

PlFS#1 corresponds to tests used in Baveno 6 rule for EV NPV (De Franchis J Hepatol 2015) but cut-offs are here specific to LEV. One can add a NPV zone (PlFS#1b).

One can use only blood tests (FM/CM or CMFM#1) with accuracy slightly superior to modified Baveno 6 rule by accepting a small proportion of missed LEV not significantly from 0% of Baveno 6 rule. Otherwise, by targeting 0% missed LEV (CMFM#1b), the spared UGIE rate is significantly decreased.

The combination of CM and VCTE allows no missed LEV but with a spared UGIE rate significantly decreased compared to the modified Baveno 6 rule.

The combination of the modified Baveno 6 rule (PlFS#1) for LEV out zone (with an additional zone) to the CM and VCTE combination for LEV in zone (PlCMFS#1) associates respective advantages with significantly increased spared UGIE rate compared to each constitutive algorithm. Additional zones for LEV in or out zone (PlFMCMFS#1) increased this rate at the expense of possible overfitting (optimism bias).

### Algorithms for Non-Invasive Diagnosis of Esophageal Varices

### Algorithm PlFS#2

This is a Platelets+VCTE (also known as Fibroscan) algorithm for esophageal varices. The EV rule out zone is defined by the following cut-offs: platelets >87 G/l and VCTE<11.9 kPa.

The EV rule in zone is defined by the following cut-offs: platelets <93 G/l and VCTE>30 kPa.

The cut-offs for NPV zone are improved compared to original Baveno 6 rule; PPV zone is also an improvement.

### Formula

With 0=NPV zone (LEV rule out zone), 1=grey zone, 2=PPV zone (LEV rule in zone).

### CMFM#1

compute CMFM#1=1.

### do if (CirrhoMeter <0.21).

compute CMFM#1=0.
else if (FibroMeter <0.78).
compute CMFM#1=0.
else if (CirrhoMeter >0.998) and (FibroMeter >0.9993).
compute CMFM#1=2.
end if.
execute.

### CMFM#1b

compute CMFM#1b=1.

### do if (CM2G<0.042).

compute CMFM#1b=0.
else if (FM2G<0.51).
compute CMFM#1b=0.
else if (CM2G>0.99945).
compute CMFM#1b=2.
end if.
execute.

### PlFS#1

compute PlFS#1=1.
do if (platelets >110) and (VCTE<26.5).
compute PlFS#1=0.
else if (platelets <45) and (VCTE>32).
compute PlFS#1=2.
end if.
execute.

### PlFS#1b

compute PlFS#1=1.
do if (platelets >110) and (VCTE<26.5).
compute PlFS#1=0.
else if (platelets <65) and (VCTE>32).
compute PlFS#1=2.
end if.
execute.

### PlFS#2

compute PlFS#2=1.
do if (platelets >87) and (VCTE<11.9).
compute PlFS#2=0.
else if (platelets <93) and (VCTE>30).
compute PlFS#2=2.
end if.
execute.

### CMFS#1

compute CMFS#1=1.

### do if (CirrhoMeter <0.6) and (VCTE<14).

compute CMFS#1=0.
else if (CirrhoMeter >0.99891) and (VCTE>55).
compute CMFS#1=2.
end if.
execute.

### PlCMFS#1

compute PlCMFS#1=1.
do if (platelets >110) and (VCTE<26.5).
compute PlCMFS#1=0.
else if (CirrhoMeter <0.334) and (VCTE<35).
compute PlCMFS#1=0.
else if (CirrhoMeter >0.99891) and (VCTE>55).
compute PlCMFS#1=2.
end if.
execute.

### PlFMCMFS#1

compute PlFMCMFS#1=1.
do if (platelets >110) and (VCTE<26.5).
compute PlFMCMFS#1=0.
else if (CirrhoMeter <0.334) and (VCTE<35).
compute PlFMCMFS#1=0.
else if (CirrhoMeter <0.04).
compute PlFMCMFS#1=0.
else if (FibroMeter <0.05).
compute PlFMCMFS#1=0.
else if (FibroMeter <0.895) and (VCTE<33).
compute PlFMCMFS#1=0.
else if (VCTE<9.1).
compute PlFMCMFS#1=0.
else if (CirrhoMeter >0.99891) and (VCTE>55).
compute PlFMCMFS#1=2.
else if (FibroMeter >0.9994) and (VCTE>60).
compute PlFMCMFS#1=2.
end if.
execute.

### Example 5: Multiple Zones of Predictive Values

### Introduction

This example describes a method to determine a diagnostic algorithm based on multiple diagnostic tests by using their respective predictive values.

The data supporting this description are drawn from the diagnosis of esophageal varices in cirrhosis.

### Aim

The objective of the method of multiple zones of predictive values is to increase the predictive value of a diagnostic algorithm by combining the predictive values of at least 3 diagnostic tests (or markers).

### Background

Frequently, diagnostic tests cannot be sorted as binary with a yes/no result and a single cut-off.

The main solution is to consider predictive values and to accept a maximal error risk, e.g. 5% even 0%. Thus, one has to calculate two cut-offs: one for negative predictive value (NPV) and one for positive predictive value (PPV).

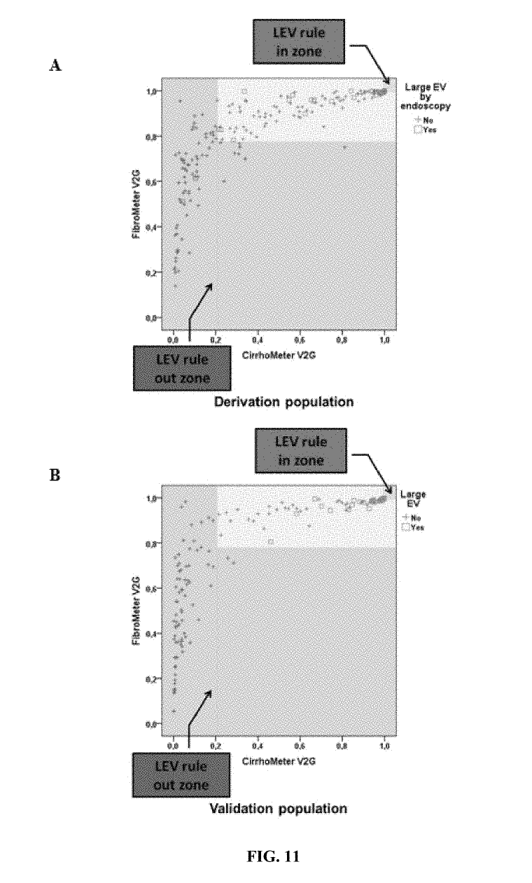

The cut-offs for a single diagnostic test are calculated as shown in FIG. 12.

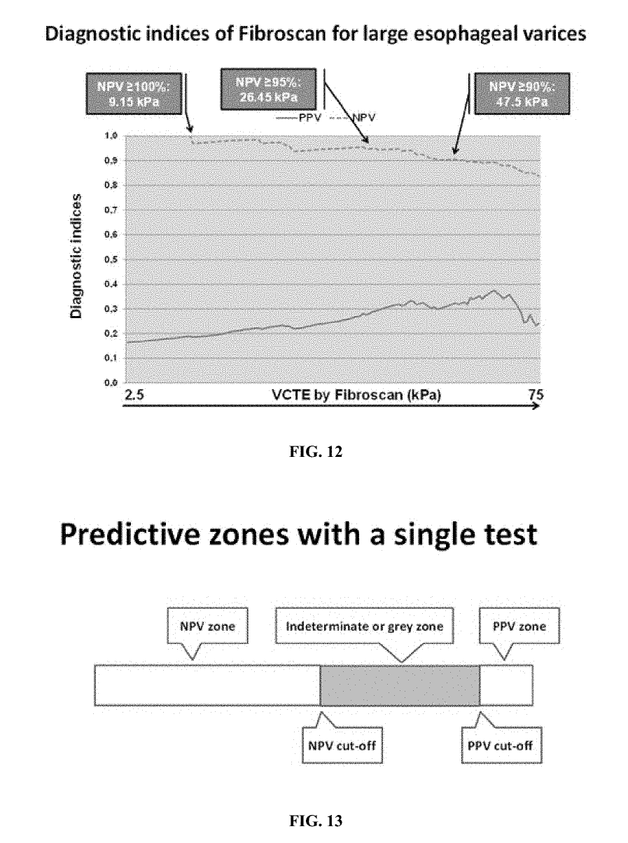

Then, the predictive zones of a single diagnostic test are obtained as shown in FIG. 13.

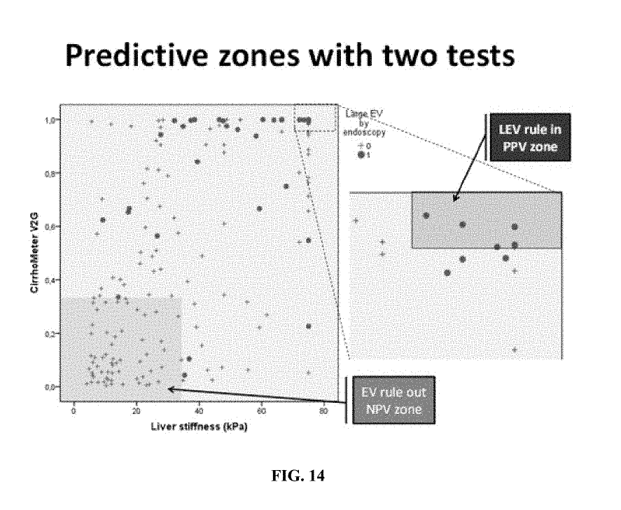

It can be more accurate to define predictive zones using two diagnostic tests as shown in FIG. 14.

### Description

It is more difficult to calculate predictive zones by using more than two tests. A new method to solve this difficulty is described below.

### Principles

The principle is the following.
In a first step, one calculates the predictive zones using the two tests having the larger predictive zones. The choice of the two tests can be done according to several classical statistical techniques, for example the most accurate tests according to multivariate analysis or correlation.
In a second step, one considers one of the 2 predictive zones, for example the NPV zone (usually, the NPV zones are larger than the PPV zones) and one excludes the patients located in the previous NPV zone (first step).
In a third step, one considers a novel test combination, i.e. at least one of the two tests is necessarily different from those used in the first step. Otherwise, the NPV zone would be empty. Then, on tries to determine a new NPV zone. If the NPV zone is empty, one considers another test combination.
In the optional 4^{th} step, the process is reiterated by excluding patients included in the second NPV zone until any new NPV zone can be found.
In the next step, the process is the same for the PPV zone as in steps 2 to 4.

### Conditions

### 1/ Plausibility

The NPV and PPV zones are determined according to classical rules described in the hereinabove background paragraph.

Thus, these zones have to be plausible, i.e. zones have to be located in the expected values of the diagnostic test for the corresponding predictive value, e.g. platelet count in the highest range to rule out (NPV zone) large esophageal varices.

### 2/Construction

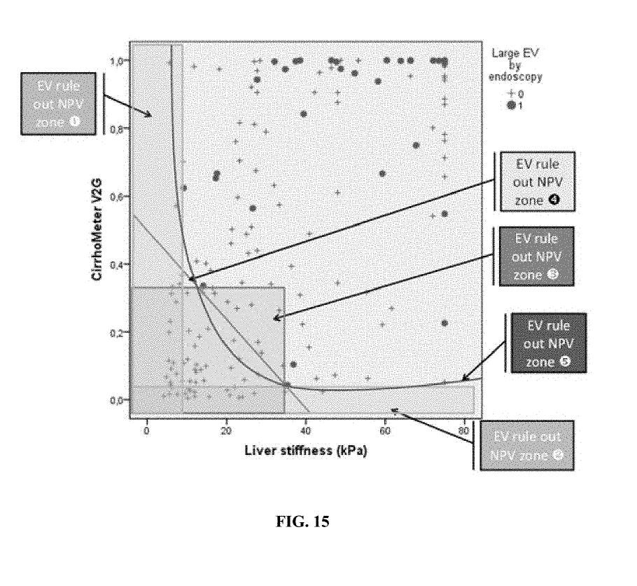

The predictive zone obtained with two tests can be calculated in several ways:
Combination of 2 predictive zones of single tests, i.e. zones 1+2 in FIG. 15 i.e. (test 1<x) or (test 2<y).
1 predictive zone of combined tests, i.e. zone 3 in FIG. 15 i.e. (test 1<z) and (test 2<v).
A combination derived from the previous ones i.e. 1+3 or 2+3 or 1+2+3.
The cut-off can be not a constant value and determined by a mathematical function of the two tests, for example by a line, e.g. test 1=a+b test 2, i.e. zone 4; or a curve, e.g. 5 in FIG. 15.
Finally, a combination derived from the previous ones, e.g. 1+2+4.

### 3/ Overfitting

This method bears the risk of maximizing the optimism bias; therefore, the following precautions have to be taken:
A strict plausibility as previously described.
A large sample size, proportional to the number of additional predictive zones.
A validation in an independent population, if possible with close characteristics, e.g. same etiology.

### Examples

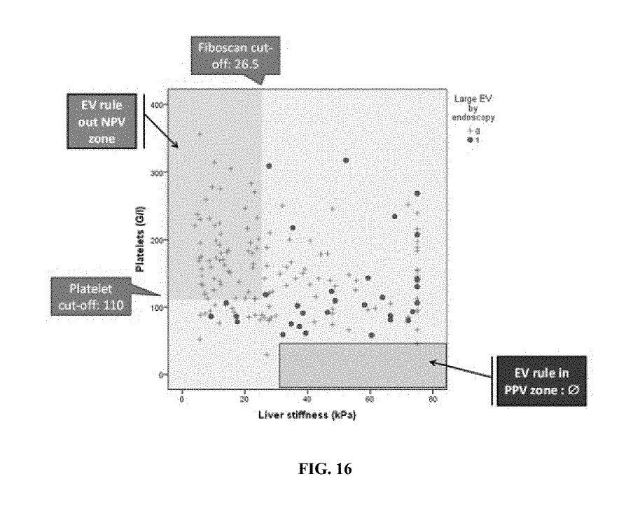

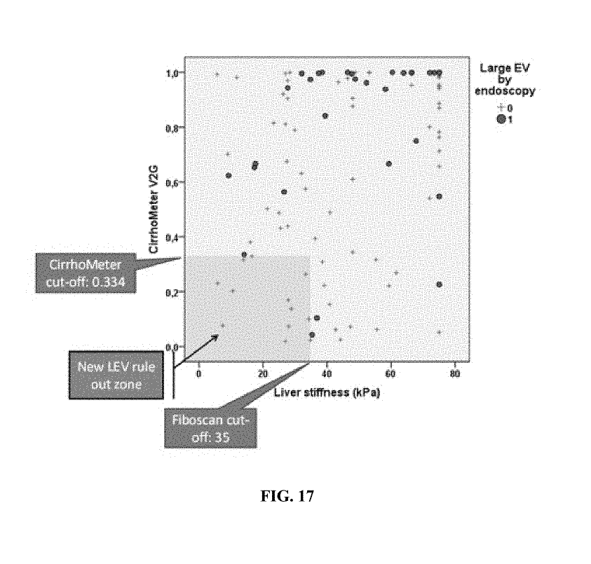

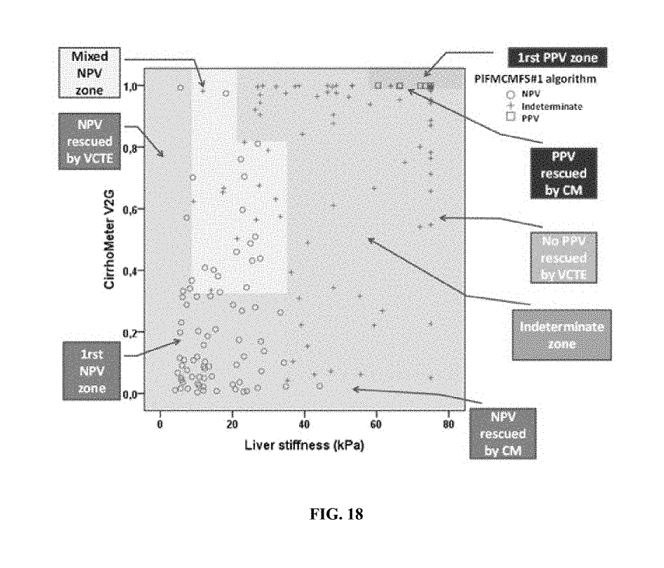

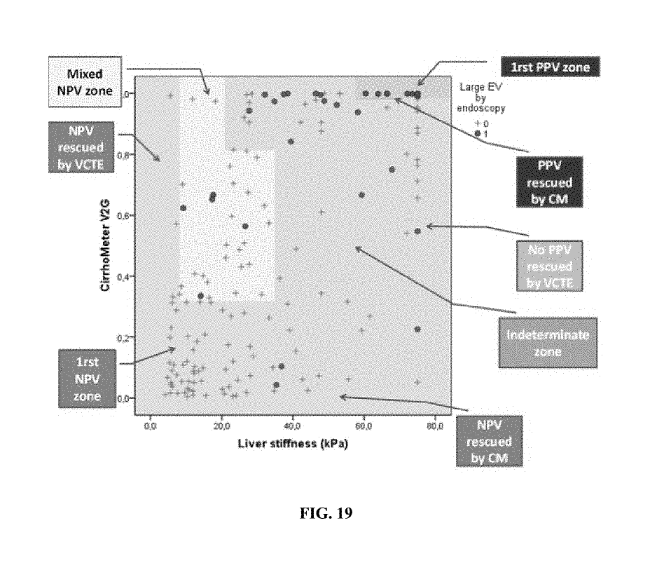

FIGS. 16 to 19 describe an algorithm, called PlFMCMFS#1, for the diagnosis of esophageal varices in cirrhosis (see Example 4 hereinabove). The first step is based on the platelet x Fibroscan combination whereas the further steps are based on pair combination among the following tests: Fibroscan, CirrhoMeter, FibroMeter.

1-16. (canceled)

17. A non-invasive method for assessing the presence and/or severity of varices, selected from gastric and esophageal varices in a liver disease patient, wherein said method comprises:
(a) carrying out at least one non-invasive test for assessing the severity of a hepatic lesion or disorder selected from the group consisting of ELF, FibroSpect™, APRI, FIB-4, Hepascore, FibroMeter™, CirrhoMeter™, CombiMeter, Elasto-FibroMeter™, Elasto-Fibrotest, and InflaMeter™, wherein said non-invasive test results in at least one value, and
(b) comparing the at least one value obtained at step (a) with cut-offs of said non-invasive test for assessing the presence and/or severity of varices, selected from gastric and esophageal varices.

18. The non-invasive method according to claim 17, wherein step a) further comprises measuring the platelet count in a blood sample from the liver disease patient.

19. The non-invasive method according to claim 17, wherein step a) comprises carrying out at least one non-invasive test for assessing the severity of a hepatic lesion or disorder selected from the group consisting of ELF, FibroSpect™, APRI, FIB-4, Hepascore, FibroMeter™, CirrhoMeter™, CombiMeter, Elasto-FibroMeter™, Elasto-Fibrotest, and InflaMeter™; carrying out another non-invasive test for assessing the severity of a hepatic lesion or disorder selected from the group consisting of ELF, FibroSpect™, APRI, FIB-4, Hepascore, FibroMeter™, CirrhoMeter™, CombiMeter, Elasto-FibroMeter™, Elasto-Fibrotest, InflaMeter™, VCTE (also known as Fibroscan), ARFI, VTE, supersonic elastometry and MRI stiffness, wherein the at least two non-invasive tests are different.

20. The non-invasive method according to claim 19, wherein step a) further comprises measuring the platelet count in a blood sample from the liver disease patient.

21. The non-invasive method according to claim 17, wherein the method is for assessing the presence of large esophageal varices.

22. The non-invasive method according to claim 17, wherein said cut-offs are a negative predictive value (NPV) cut-off and a positive predictive value (PPV) cut-off, or a sensitivity cut-off and a specificity cut-off, and wherein said NPV and PPV cut-offs define two predictive zones, a NPV predictive zone and a PPV predictive zone.

23. The non-invasive method according to claim 22, wherein:
one or more value obtained in step (a) below the NPV cut-off or below the sensitivity cut-off is in the NPV predictive zone and is indicative of the absence of varices, selected from gastric and esophageal varices in the patient, and
one or more value obtained in step (a) above the PPV cut-off or above the specificity cut-off is in the PPV predictive zone and is indicative of the presence of varices, selected from gastric and esophageal varices in the patient.

24. The non-invasive method according to claim 22, wherein, if the value obtained in step (a) is in the indeterminate zone between the NPV cut-off and the PPV cut-off or between the sensitivity cut-off and the specificity cut-off, then the method further comprises one or more repetition(s) of step (a) and step (b) wherein at least one non-invasive test carried out for assessing the severity of a hepatic lesion or disorder is different from the at least one non-invasive test previously carried out, thereby defining new NPV and PPV predictive zones and assessing the presence and/or severity of varices in said patient through the use of multiple NPV and PPV predictive zones.

25. The non-invasive method according to claim 22, wherein, if the value obtained in step (a) is in the indeterminate zone between the NPV cut-off and the PPV cut-off or between the sensitivity cut-off and the specificity cut-off, then the method further comprises the steps of:
(c) measuring at least one of the following variables from the subject:
biomarkers,
clinical data,
binary markers,
physical data from medical imaging or clinical measurement
(d) obtaining imaging data on varices status, wherein said imaging data are obtained by a non-invasive imaging method,
(e) mathematically combining:
the variables obtained in step (c), or any mathematical combination thereof, with
the data obtained at step (d),
wherein the mathematical combination results in a diagnostic score, and
(f) assessing the presence and/or severity of varices, selected from gastric and esophageal varices based on the diagnostic score obtained in step (e).

26. The non-invasive method according to claim 25, wherein at step (d) the imaging data on varices status are obtained by a non-invasive imaging method or by a radiology method.

27. The non-invasive method according to claim 25, wherein at step (d) the imaging data on varices status are obtained by esophageal capsule endoscopy.

28. The non-invasive method according to claim 25, wherein at step (c), the obtained variables are the variables of the non-invasive test carried out in step (a), and wherein at step (d) the imaging data on varices status are obtained by a non-invasive imaging method or by a radiology method.

29. The non-invasive method according to claim 17, wherein the at least one non-invasive test carried out in step (a) is a CirrhoMeter.

30. The non-invasive method according to claim 25, wherein the at least one non-invasive test carried out in step (a) is a CirrhoMeter, and wherein the variables obtained at step (c) are the variables of a CirrhoMeter.

31. The non-invasive method according to claim 17, wherein the patient is affected with a chronic hepatic disease selected from the group consisting of chronic viral hepatitis C, chronic viral hepatitis B, chronic viral hepatitis D, chronic viral hepatitis E, non-alcoholic fatty liver disease (NAFLD), alcoholic chronic liver disease, autoimmune hepatitis, primary biliary cirrhosis, hemochromatosis and Wilson disease.

32. The non-invasive method according to claim 17, wherein the patient is a cirrhotic patient.

33. A non-invasive method for assessing the presence and/or severity of varices, selected from gastric and esophageal varices in a hepatic disease patient, wherein said method comprises:
i. measuring at least one of the following variables from the subject:
biomarkers,
clinical data,
binary markers,
physical data from medical imaging or clinical measurement,
ii. obtaining imaging data on varices status, wherein said imaging data are obtained by a non-invasive imaging method,
iii. mathematically combining:
the variables obtained in step (i), or any mathematical combination thereof, with
the data obtained at step (ii),
wherein the mathematical combination results in a diagnostic score, and
iv. assessing the presence and/or severity of varices, selected from gastric and esophageal varices based on the diagnostic score obtained in step (iii).

34. The non-invasive method according to claim 17, wherein the patient was previously diagnosed as cirrhotic, or wherein the patient previously obtained a value between the NPV and the PPV cut-offs in a method wherein said cut-offs are a negative predictive value (NPV) cut-off and a positive predictive value (PPV) cut-off, or a sensitivity cut-off and a specificity cut-off, and wherein said NPV and PPV cut-offs define two predictive zones, a NPV predictive zone and a PPV predictive zone.

35. A microprocessor comprising a computer algorithm carrying out the method according to claim 17.
